(19) United States
(12) Patent Application Publication
(10) Pub. No.: US 2021/0386811 A1
(43) Pub. Date: Dec. 16, 2021

(54) COMPOSITIONS AND METHODS FOR PROMOTING AND MAINTAINING ORAL HEALTH

(71) Applicant: StellaLife, Inc., Northbrook, IL (US)

(72) Inventor: Walter Tatch, Northbrook, IL (US)

(21) Appl. No.: 17/282,430

(22) PCT Filed: Oct. 4, 2019
(86) PCT No.: PCT/US2019/054607

§ 371 Date: Apr. 2, 2021

Related U.S. Application Data
(60) Provisional application No. 62/793,487, filed on Jan. 17, 2019.
(60) Provisional application No. 62/766,591, filed on Oct. 4, 2018.
(60) Provisional application No. 62/838,422, filed on Apr. 25, 2019.

(51) Int. Cl. A61K 36/28; A61K 36/56; A61K 36/45; A61K 36/185; A61K 45/06

(52) U.S. Cl. CPC A61K 36/28; A61K 36/56; A61K 45/06; A61K 36/185; A61K 36/45

(57) ABSTRACT

The present disclosure relates to compositions and methods for the promotion and maintenance of oral health. Specifically, the present disclosure relates to pain management, promoting healing, reducing post-surgical edemas, reducing the need for the opioids in post-surgical pain management, treating various inflammatory conditions in the oral cavity, general maintenance of a healthy oral cavity, and general dental care. The present disclosure presents compositions and methods for promoting and maintaining oral health, including decreasing pain, inflammation, swelling, and bruising associated with dental procedures, and accelerating recovery from dental procedures and other oral procedures or trauma.

### CROSS REFERENCE TO RELATED APPLICATIONS

This application claims the benefit of: U.S. Provisional Patent Application No. 62/766,591, filed Oct. 4, 2018, entitled METHOD OF USING HOMEOPATHIC KIT CONTAINING GEL, RINSE AND SPRAY FOR MANAGEMENT OF POST-ORAL SURGICAL RECOVERY AND MAINTENANCE OF ORAL HEALTH; U.S. Provisional Patent Application No. 62/793,487, filed Jan. 17, 2019, entitled COMPOSITIONS AND METHODS FOR MANAGEMENT OF POST-SURGICAL ORAL RECOVERY AND MAINTENANCE OF ORAL HEALTH; U.S. Provisional Patent Application No. 62/838,422, filed Apr. 25, 2019, entitled COMPOSITIONS AND METHODS FOR MANAGEMENT OF POST-SURGICAL ORAL RECOVERY AND MAINTENANCE OF ORAL HEALTH; the contents of which are each incorporated herein by reference in their entirety.

### FIELD OF THE DISCLOSURE

The present disclosure relates to compositions and methods for the promotion and maintenance of oral health. Specifically, the present disclosure relates to pain management, promoting healing, reducing post-surgical edemas, reducing the need for the opioids in post-surgical pain management, treating various inflammatory conditions in the oral cavity, general maintenance of a healthy oral cavity, and general dental care. The present disclosure presents compositions and methods for promoting and maintaining oral health, including decreasing pain, inflammation, swelling, and bruising associated with dental procedures, and accelerating recovery from dental procedures and other oral procedures or trauma.

### BACKGROUND

Oral health is an important component of physical well-being. According to The World Oral Health Report, oral health implies being free of chronic oro-facial pain, oral and pharyngeal (throat) cancer, oral tissue lesions, birth defects such as cleft lip and palate, and other diseases and disorders that affect the oral, dental and craniofacial tissues, collectively known as the craniofacial complex. Proper oral health can reduce premature mortality and disease (periodontal disease is known to be associated with diabetes).

Patients currently suffer from a number of inflammatory conditions in the oral cavity for which there is presently no effective treatment or solution. A single treatment has not yet been identified to possess a combination of: antimicrobial properties, a reduction in both swelling and pain, improved post-surgical recovery, and a reduction in the anxiety associated with surgery. Patients taking powerful pain-relief medications such as VICODIN® (Hydrocodone/Acetaminophen, referred to herein as NORCO™ pills), are typically unable to return to everyday activities, including school and work, over the time course of treatment. Likewise, the over prescription of narcotic pain medications, together with their high potential for abuse, makes them an unattractive therapeutic.

There is therefore a long-felt need in the art for improved compositions and methods to promote wound healing, reduce inflammation, and mitigate post-surgical pain and trismus following oral surgeries or other invasive oral procedures. There is also a long felt need in the art for improved compositions which circumvent or reduce the need for opioid prescriptions.

### SUMMARY OF THE DISCLOSURE

The present disclosure presents compositions for the promotion and maintenance of oral health. In certain embodiments, the composition includes: an attenuation of Arnica montana; an attenuation of Calendula officinalis; an attenuation of Chamomilla; an attenuation of Ignatia amara; an attenuation of Ledum palustre; an attenuation of Silicea; and at least one of an attenuation of Echinacea purpurea or an attenuation of Echinacea augustifolia.

In certain embodiments, the composition includes one or more ingredients selected from: an attenuation of Azadirachta indica; an attenuation of Hepar sulphuris calcareum; an attenuation of Hypericum; an attenuation of Mercurius vivus; an attenuation of Ruta graveolens; an attenuation of Plantago major; an attenuation of Romains officinalis; an attenuation of Origanum vulgare L; an attenuation of Thymus vulgaris L; an attenuation of Lavandula angustifolia/officinalis; an attenuation of Salvia officinalis; an attenuation of Melissa officinalis; an attenuation of Cuminum cyminum; an attenuation of Petroselinum crispum; an attenuation of Calendula officinalis; an attenuation of Tagetes erecta; an attenuation of Boswellia sacra; an attenuation of Sambucus nigra; an attenuation of Copaifera langsdorfii; an attenuation of Curcuma longa; an attenuation of Allium sativu; an attenuation of Punica granatum; an attenuation of Euterpe oleracea; an attenuation of Sophora flavescens; an attenuation of Rheum rhabarbarum; an attenuation of Fagopyrum esculentum; an attenuation of Camellia sinensis; an attenuation of Coptis teeta; an attenuation of Hydrastis canadensis; an attenuation of Mahonia aquifolium; an attenuation of Phellodendron amurense; an attenuation of Berberis vulgaris; an attenuation of Xanthorhiza simplicissima; an attenuation of Lonicera ceprifoliu; an attenuation of Vaccinium macrocarpon; an attenuation of Cinnamomum zeylanicum Nees; an attenuation of Cinnamomum verum; an attenuation of Vitis Vinifera; an attenuation of Terminalia Billerica; an attenuation of Pinus Pinaster; an attenuation of Albizia Lebbek; an attenuation of Melia azadirachta; an attenuation of Salvadora persica; an attenuation of Paullinia cupana; an attenuation of Piper betle; an attenuation of Syzygium aromaticum; an attenuation of Commiphora myrrha; an attenuation of Juglans regia; an attenuation of Scutellaria baicalensis; an attenuation of Magnolia officinalis; an attenuation of Origanum vulgare; an attenuation of Origanum onites; an attenuation of Origanum majorana; an attenuation of Origanum heracleoticum; an attenuation of Thymus citriodorus; an attenuation of Thymus pulegioides; an attenuation of Thymus x herba-barona; an attenuation of Thymus serpyllum; an attenuation of Lavandula stoechas; an attenuation of Lavandula dentate; an attenuation of Lavandula x intermedia; an attenuation of Lavandula multifida; an attenuation of Salvia divinorum; an attenuation of Salvia apiana; an attenuation of Calendula arvensis; an attenuation of Calendula maderensis; an attenuation of Tagetes minuta; an attenuation of Tagetes patula; an attenuation of Boswellia frereana; an attenuation of Boswellia serrata; an attenuation of Boswellia papyrifera; an attenuation of Sambucus melanocarpa; an attenuation of Sambucus racemosa; an attenuation of Rheum rhaponticum; an attenuation of Citrullus colocynthis; and an attenuation of Cucumis sativus.

In certain embodiments, at least one of the ingredients in the composition is substituted with an ingredient selected from: an attenuation of Acontium nap; an attenuation of Gelsemium; an attenuation of Apis mellifica; an attenuation of Cantharis; an attenuation of Rhus toxicodendron; and an attenuation of Symphytum officinale.

In certain embodiments, the composition includes an attenuation of Azadirachta indica. In certain embodiments, the composition includes an attenuation of Hepar sulphuris calcareum. In certain embodiments, the composition includes an attenuation of Hypericum. In certain embodiments, the composition includes an attenuation of Mercurius vivus. In certain embodiments, the composition includes an attenuation of Ruta graveolens. In certain embodiments, the composition includes attenuation of Plantago major. In some embodiments, the attenuation of Gelsemium is substituted for the attenuation of Silicea.

In certain embodiments, the composition includes an inert substance or inactive ingredient.

In certain embodiments, the composition includes at least one of: an allopathic medicine, a vitamin, a mineral, and an amino acid. In certain embodiments, the composition a mineral. In certain embodiments, the mineral is selected from: Antimony, Barium, Beryllium, Bismuth, Boron, Bromine, Cadmium, Calcium, Carbonate, Cerium, Cesium, Chloride, Chromium, Cobalt, Copper, Dysprosium, Erbium, Europium, Gadolinium, Gallium, Germanium, Gold, Hafnium, Holmium, Indium, Iodine, Iridium, Iron, Lanthanum, Lead, Lithium, Lutetium, Magnesium, Manganese, Molybdenum, Neodymium, Nickel, Niobium, Osmium, Palladium, Propolis, Phosphorus, Platinum, Potassium, Praseodymium, Rhenium, Rhodium, Rubidium, Ruthenium, Samarium, Scandium, Selenium, Silicon, Silver, Sodium, Strontium, Sulfate/Sulfur, Tantalum, Tellurium, Terbium, Thallium, Thorium, Thulium, Tin, Titanium, Tungsten, Vanadium, Ytterbium, Yttrium, Zinc, and Zirconium. In certain embodiments, propolis is an inactive ingredient in the composition.

In certain embodiments, the composition includes an excipient selected from: viscosity modifying agents, buffers, antioxidants, emulsifying agents, absorbents, antiacne agents, antiperspirants, anticaking agents, antifoaming agents, antimicrobial agents, anti-infective agents, antioxidants, antidandruff agents, astringents, binders, buffers, biological additives, buffering agents, bulking agents, chelating agents, chemical additives, coupling agents, conditioners, colorants, cosmetic astringents, cosmetic biocides, denaturants, drug astringents, detergents, dispersants, external analgesics, film formers, foaming agents, fragrance components, flavoring agents humectants, keratolytics, opacifying agents, pH adjusters, preservatives, propellants, proteins, retinoids, reducing agents, sequestrants, skin bleaching agents, skin-conditioning agents (humectants, miscellaneous, and occlusive), skin soothing agents, skin healing agents, softeners, solubilizing agents, lubricants, penetrants, plasticizers, solvents and co-solvents, sun screening additives, sweeteners, salts, essential oils, and vitamins. In certain embodiments, the mineral or the excipient is within the range of 0.01% to 95% of the total weight (wt %) of the composition. In certain embodiments, the composition includes a solvent selected from: alcohol, water, water-alcohol mixture, glycerin, and isotonic sodium chloride solution.

In certain embodiments, at least one ingredient of the composition is formulated in a dilution within the range of 1X to 40X. In certain embodiments, at least one ingredient of the composition is formulated in a dilution 1X, 2X, 3X, 4X, 5X, 6X, 7X, 8X, 9X, 10X, 11X, 12X, 13X, 14X, 15X, 16X, 17X, 18X, 19X, 20X, 21X, 22X, 23X, 24X, 25X, 26X, 27X, 28X, 29X, 30X, 31X, 32X, 33X, 34X, 35X, 36X, 37X, 38X, 39X, or 40X.

In certain embodiments, the composition is formulated as a gel, a rinse, a spray, a balm, a pellet, a pill, or a drop. In certain embodiments, the composition is formulated as a gel. In certain embodiments, the composition is formulated as a spray, such as a sublingual spray.

In certain embodiments, the composition is formulated as a gel. In certain embodiments, the gel includes an attenuation of Arnica montana in a dilution of 3X, 6X, or 18X. In certain embodiments, the gel includes an attenuation of Calendula officinalis with a dilution of 3X. In certain embodiments, the gel includes an attenuation of Chamomilla with a dilution of 5X. In certain embodiments, the gel includes an attenuation of Echinacea angustifolia or Echinacea purpurea with a dilution of 3X. In certain embodiments, the gel includes an attenuation of Azadirachta indica with a dilution of 1X. In certain embodiments, the gel includes an attenuation of Ignatia amara with a dilution of 30X. In certain embodiments, the gel includes an attenuation of Ledum palustre with a dilution of 6X. In certain embodiments, the gel includes an attenuation of Ruta graveolens with a dilution of 12X. In certain embodiments, the gel includes an attenuation of Mercurius vivus with a dilution of 10X. In certain embodiments, the gel includes an attenuation of Hypericum with a dilution of 10X. In certain embodiments, the gel includes an attenuation of Silicea with a dilution of 8X. In certain embodiments, the composition includes at least one ingredient selected from: allantoin, hydroxyethyl cellulose, peppermint oil, purified water, sodium benzoate, and xylitol.

In certain embodiments, the composition is formulated as a spray, such as a sublingual spray. In certain embodiments, the spray includes an attenuation of Arnica montana in a dilution of 3X, 6X, or 18X. In certain embodiments, the gel includes an attenuation of Calendula officinalis with a dilution of 3X. In certain embodiments, the gel includes an attenuation of Chamomilla with a dilution of 5X. In certain embodiments, the gel includes an attenuation of Echinacea angustifolia or Echinacea purpurea with a dilution of 3X. In certain embodiments, the gel includes an attenuation of Azadirachta indica with a dilution of 1X. In certain embodiments, the gel includes an attenuation of Ignatia amara with a dilution of 30X. In certain embodiments, the gel includes an attenuation of Ledum palustre with a dilution of 6X. In certain embodiments, the gel includes an attenuation of Ruta graveolens with a dilution of 12X. In certain embodiments, the gel includes an attenuation of Mercurius vivus with a dilution of 10X. In certain embodiments, the gel includes an attenuation of Hypericum with a dilution of 10X. In certain embodiments, the gel includes an attenuation of Silicea with a dilution of 8X. In certain embodiments, the composition includes at least one ingredient selected from: citric acid, Glycerin USP, potassium sorbate, and water.

In certain embodiments, the composition is formulated as a spray. In certain embodiments, the spray includes an attenuation of Arnica montana with a dilution of 3X, 6X, or 18X. In certain embodiments, the spray includes an attenuation of Calendula officinalis with a dilution of 3X. In certain embodiments, the spray includes an attenuation of Chamomilla with a dilution of 5X. In certain embodiments, the spray includes an attenuation of Echinacea angustifolia or Echinacea purpurea with a dilution of 3X. In certain embodiments, the spray includes an attenuation of Azadirachta indica with a dilution of 1X. In certain embodiments, the spray includes an attenuation of Ignatia amara with a dilution of 30X. In certain embodiments, the spray includes an attenuation of Hepar sulphuris calcareum with a dilution of 8X. In certain embodiments, the spray includes an attenuation of Aconitum napellus with a dilution of 3X. In certain embodiments, the spray includes an attenuation of Echinacea angustifolia with a dilution of 3X. In certain embodiments, the spray includes an attenuation of Ruta graveolens with a dilution of 12X. In certain embodiments, the spray includes an of Mercurius vivus with a dilution of 10X. In certain embodiments, the spray includes an attenuation of Hypericum with a dilution of 10X. In certain embodiments, the spray includes an attenuation of Gelsemium with a dilution of 30X. In certain embodiments, the spray includes an attenuation of Silicea with dilution of 8X.

In certain embodiments, the composition is formulated as drops. In certain embodiments, the drops includes an attenuation of Arnica montana with a dilution of 3X, 6X, or 18X. In certain embodiments, the drops includes an attenuation of Calendula officinalis with a dilution of 3X. In certain embodiments, the drops includes an attenuation of Chamomilla with a dilution of 5X. In certain embodiments, the drops includes an attenuation of Echinacea purpurea with a dilution of 3X. In certain embodiments, the drops includes an attenuation of Azadirachta indica with a dilution of 1X. In certain embodiments, the drops includes an attenuation of Ignatia amara with a dilution of 30X. In certain embodiments, the drops includes an attenuation of Hepar sulphuris calcareum with a dilution of 8X. In certain embodiments, the drops includes an attenuation of Aconitum napellus with a dilution of 3X. In certain embodiments, the drops includes an attenuation of Echinacea angustifolia with a dilution of 3X. In certain embodiments, the drops includes an attenuation of Ruta graveolens with a dilution of 12X. In certain embodiments, the drops includes an attenuation of Mercurius vivus with a dilution of 10X. In certain embodiments, the drops includes an attenuation of Hypericum with a dilution of 10X. In certain embodiments, the drops includes an attenuation of Gelsemium with a dilution of 30X. In certain embodiments, the drops includes an attenuation of Silicea with dilution of 8X. In certain embodiments, the composition further includes a bupivacaine liposome injectable suspension. In certain embodiments, the composition includes at least one ingredient selected from: citric acid, glycerin, potassium sorbate, and purified water.

The present disclosure presents a composition which includes: an attenuation of Calendula officinalis; an attenuation of Echinacea purpurea; an attenuation of Plantago major; and an attenuation of Azadirachta indica. In certain embodiments, the composition is formulated as a rinse. In certain embodiments, the rinse includes an attenuation of Calendula officinalis with a dilution of 1X. In certain embodiments, the rinse includes an attenuation of Echinacea purpurea with a dilution of 1X. In certain embodiments, the rinse includes an attenuation of Plantago major with a dilution of 1X. In certain embodiments, the composition includes at least one ingredient selected from: Xylitol, Allantoin, Peppermint oil, Glycerin, Citric acid, potassium sorbate, Purified Water, Polysorbate 20, and propolis.

The present disclosure presents a method of reducing the depth of a periodontal pocket in a subject. In certain embodiments, the method includes administering or applying to the subject a composition described herein. In certain embodiments, the depth is reduced by at least 2 mm. In certain embodiments, the depth is reduced to within a range of about 3 mm to about 4 mm. In certain embodiments, the depth is reduced to about 3 mm. In certain embodiments, the depth is reduced to about 4 mm.

The present disclosure presents a method of preventing or reducing pain or inflammation in a subject which includes administering or applying to the subject a composition described herein, thereby preventing or reducing pain in the subject.

The present disclosure presents a method of treating oral aphthous ulcer in a subject which includes administering or applying to the subject the composition described herein.

The present disclosure presents a method of accelerating the rate of healing in a subject which includes administering or applying to the subject a composition described herein, thereby accelerating the rate of healing in the subject compared to prior to administering or applying.

The present disclosure presents a method of reducing bacteria in the oral cavity of a subject. In certain embodiments, the method includes administering or applying to the subject a composition described herein, thereby reducing bacteria in the oral cavity of the subject compared to prior to administering or applying. In certain embodiments, the method includes administering an opioid to the patient. In certain embodiments, the administering or applying occurs once, twice, three times, four times, or five times per day. In certain embodiments, administering or applying begins five days before a surgery. In certain embodiments, administering or applying begins less than five days before a surgery. In certain embodiments, administering or applying begins five days, four days, three days, two days, or one day before a surgery. In certain embodiments, administering or applying occurs daily until the day of the surgery. In certain embodiments, administering or applying begins seven days, six days, five days, four days, three days, two days, or one day after a surgery. In certain embodiments, administering or applying occurs at least daily up to seven days after a surgery.

The present disclosure presents a combination therapy of a gel described herein and a rinse described herein. In certain embodiments, the combination therapy includes drops as described herein. In certain embodiments, the combination therapy includes a spray described herein.

The present disclosure presents a method of treating an inflammatory autoimmune condition of the oral cavity in a subject which includes administering or applying to the subject one of the combination therapies described herein. In certain amendments, the inflammatory autoimmune condition is selected from: Erosive Lichen Planus, Pemphigoid, and Pemphigus.

In certain embodiments, compositions of the present disclosure are used for oral and/or dental care. Oral and/or dental care includes, for example, general oral maintenance; reduction of flora in the mouth; reduction of pain; reduction of inflammation; reduction of bruising, trismus, recovery after dental surgeries; amelioration of anxiety associated with dental care or preventative measures for maladies of the oral cavity. Maladies of the oral cavity include, for example, cavities/tooth decay, bone loss, and periodontal pocket formation. In a healthy mouth, periodontal pockets should not be more than 3-4 mm or proper care is not manageable. To that end, periodontal disease can occur when periodontal pockets are 5-7 mm deep.

The present disclosure presents compositions designed to decrease post-operative swelling, bruising, and pain, as well as, pre-operative and post-operative anxiety. In certain embodiments, the composition is administered or applied without use of injections and without the need for visits with a practitioner. Patients can administer or apply the compositions and formulations thereof themselves, which improves patient compliance, thereby encouraging positive medical outcomes. In some embodiments, three formulations of the composition(s) are administered or applied through different routes to act synergistically both locally and systemically to maximally benefit patient health.

In certain embodiments, the composition described herein is in a formulation that alleviates pain, inflammation, and/or trismus with an administration or application regimen that improves patient compliance as compared to other administration or application regimens. In certain embodiments, the composition described herein is in a formulation of a composition as described herein that alleviates pain, inflammation and/or trismus, while reducing or preventing anxiety associated with surgery, with an administration or application regimen that improves patient compliance. In certain embodiments, the composition described herein is in a formulation of a composition as described herein with no side effects, that is, the composition is free from any toxicity, toxic residue, and/or irritation when regularly used. In certain embodiments, a method is provided for alleviating pain, inflammation and/or trismus, and anxiety with an administration or application regimen that improves patient compliance compared to alternative administration or application regimens.

In certain embodiments, a kit is provided which includes one or more different formulations of the compositions described herein. In certain embodiments, the formulations in the kit are used to alleviate pain, inflammation, and/or trismus with an administration or application regimen to improve patient compliance compared to alternative administration or application regimens. In certain embodiments, the kit is designed to help patients recover more quickly after dental and/or surgical procedures. In certain embodiments, a kit includes different formulations of at least one composition described herein is provided to alleviate pain, inflammation and/or trismus, and anxiety with an administration or application regimen to improve patient compliance compared to alternative administration or application regimens. In certain embodiments, a kit is designed to reduce the necessity of opioid consumption after dental and/or surgical procedures. In certain embodiments, a kit is designed to help patients recover after dental and/or oral surgical procedures, while reducing the anxiety associated with surgery. In certain embodiments, a kit includes one or more of the sprays, pellets, pills, gels, pastes, balms or rinses of the present disclosure. In certain embodiments, the kit includes a gel, drops or a spray, and a rinse administered or applied through different routes to act at both local and systemic levels to maximally benefit patient health.

The present disclosure presents a formulation for maintaining oral care and for the prevention of bone loss, tooth decay, cavities, and loss of teeth. In certain embodiments, the compositions disclosed herein may be combined with other treatment methods and substances including allopathic medicines, vitamins, minerals, amino acids, traditional remedies, and inert substances for use in the methods described herein. In certain embodiments, the composition disclosed herein has antimicrobial properties.

In certain embodiments, formulations or compositions of the present disclosure are combined with other formulations. In certain embodiments, more than one formulation may be administered or applied to a subject as a combination therapy. In certain embodiments, the formulation of the composition include attenuations of one or more of the following: Arnica montana, Calendula officinalis, Chamomilla, Echinacea angustifolia, Echinacea purpurea, Ignatia amara, Hepar sulphuris calcareum, Mercurius vivus, Hypericum, Ruta graveolens, Plantago major, Apis mellifica, Cantharis, Rhus toxicodendron, Symphytum officinale, Ledum palustre (also known as marsh Labrador tea, northern Labrador tea or wild rosemary), and Silicea, or any combination thereof. In certain embodiments, the formulation of the composition includes an attenuation of at least one of the following: Arnica montana, Calendula officinalis, Chamomilla, Echinacea angustifolia, Echinacea purpurea, Ignatia amara, Hepar sulphuris calcareum, Mercurius vivus, Hypericum, Ruta graveolens, Plantago major, Apis mellifica, Cantharis, Rhus toxicodendron, Symphytum officinale, Ledum palustre (also known as marsh Labrador tea, northern Labrador tea or wild rosemary), Silicea, Aconitum napellus, and Gelsemium, or any combination thereof. Certain embodiments of the formulation includes a composition of attenuations of the following: Arnica montana, Calendula officinalis, Chamomilla, Echinacea angustifolia, Echinacea purpurea, Ignatia amara, Hepar sulphuris calcareum, Mercurius vivus, Hypericum, Ruta graveolens, Plantago major, Apis mellifica, Cantharis, Rhus toxicodendron, Symphytum officinale, Ledum palustre, Aconitum napellus, Gelsemium, and Silicea, or any combination thereof. In certain embodiments, the formulation includes a composition of attenuations of at least one of Arnica montana, Calendula officinalis, Chamomilla, Echinacea angustifolia, Echinacea purpurea, Ignatia amara, Hepar sulphuris calcareum, Mercurius vivus, Hypericum, Ruta graveolens, Plantago major, Apis mellifica, Cantharis, Rhus toxicodendron, Symphytum officinale, Ledum palustre, and Silicea. In certain embodiments, the formulation includes a composition of attenuations of at least 2 of Arnica montana, Calendula officinalis, Chamomilla, Echinacea angustifolia, Echinacea purpurea, Azadirachta indica, Ignatia amara, Hepar sulphuris calcareum, Mercurius vivus, Hypericum, Ruta graveolens, Plantago major, Apis mellifica, Cantharis, Rhus toxicodendron, Symphytum officinale, Ledum palustre, and Silicea. In certain embodiments, the formulation includes a composition of attenuations of at least 3 of Arnica montana, Calendula officinalis, Chamomilla, Echinacea angustifolia, Echinacea purpurea, Azadirachta indica, Ignatia amara, Hepar sulphuris calcareum, Mercurius vivus, Hypericum, Ruta graveolens, Plantago major, Apis mellifica, Cantharis, Rhus toxicodendron, Symphytum officinale, Ledum palustre, and Silicea. In certain embodiments, the formulation includes a composition of attenuations of at least 4 of Arnica montana, Calendula officinalis, Chamomilla, Echinacea angustifolia, Echinacea purpurea, Azadirachta indica, Ignatia amara, Hepar sulphuris calcareum, Mercurius vivus, Hypericum, Ruta graveolens, Plantago major, Apis mellifica, Cantharis, Rhus toxicodendron, Symphytum officinale, Ledum palustre, and Silicea. In certain embodiments, the formulation includes a composition of attenuations of at least 5 of Arnica montana, Calendula officinalis, Chamomilla, Echinacea angustifolia, Echinacea purpurea, Azadirachta indica, Ignatia amara, Hepar sulphuris calcareum, Mercurius vivus, Hypericum, Ruta graveolens, Plantago major, Apis mellifica, Cantharis, Rhus toxicodendron, Symphytum officinale, Ledum palustre, and Silicea. In certain embodiments, the formulation includes a composition of attenuations of at least 6 of Arnica montana, Calendula officinalis, Chamomilla, Echinacea angustifolia, Echinacea purpurea, Azadirachta indica, Ignatia amara, Hepar sulphuris calcareum, Mercurius vivus, Hypericum, Ruta graveolens, Plantago major, Apis mellifica, Cantharis, Rhus toxicodendron, Symphytum officinale, Ledum palustre, and Silicea. In certain embodiments, the formulation includes a composition of attenuations of at least 7 of Arnica montana, Calendula officinalis, Chamomilla, Echinacea angustifolia, Echinacea purpurea, Azadirachta indica, Ignatia amara, Hepar sulphuris calcareum, Mercurius vivus, Hypericum, Ruta graveolens, Plantago major, Apis mellifica, Cantharis, Rhus toxicodendron, Symphytum officinale, Ledum palustre, and Silicea. In certain embodiments, the formulation includes a composition of attenuations of at least 8 of Arnica montana, Calendula officinalis, Chamomilla, Echinacea angustifolia, Echinacea purpurea, Azadirachta indica, Ignatia amara, Hepar sulphuris calcareum, Mercurius vivus, Hypericum, Ruta graveolens, Plantago major, Apis mellifica, Cantharis, Rhus toxicodendron, Symphytum officinale, Ledum palustre, and Silicea. In certain embodiments, the formulation includes a composition of attenuations of at least 9 of Arnica montana, Calendula officinalis, Chamomilla, Echinacea angustifolia, Echinacea purpurea, Azadirachta indica, Ignatia amara, Hepar sulphuris calcareum, Mercurius vivus, Hypericum, Ruta graveolens, Plantago major, Apis mellifica, Cantharis, Rhus toxicodendron, Symphytum officinale, Ledum palustre, and Silicea. In certain embodiments, the formulation includes a composition of attenuations of at least 10 of Arnica montana, Calendula officinalis, Chamomilla, Echinacea angustifolia, Echinacea purpurea, Azadirachta indica, Ignatia amara, Hepar sulphuris calcareum, Mercurius vivus, Hypericum, Ruta graveolens, Plantago major, Apis mellifica, Cantharis, Rhus toxicodendron, Symphytum officinale, Ledum palustre, and Silicea. In certain embodiments, the formulation includes a composition of attenuations of at least 11 or more of Arnica montana, Calendula officinalis, Chamomilla, Echinacea angustifolia, Echinacea purpurea, Azadirachta indica, Ignatia amara, Hepar sulphuris calcareum, Mercurius vivus, Hypericum, Ruta graveolens, Plantago major, Apis Cantharis, Rhus toxicodendron, Symphytum officinale, Ledum palustre, and Silicea.

In some embodiments, the formulation includes a composition of attenuations of Arnica montana, Calendula officinalis, Chamomilla, Echinacea angustifolia, and Ignatia amara. In certain embodiments, the formulation includes a composition of attenuations of Arnica montana, Calendula officinalis, Chamomilla, Echinacea purpurea, and Ignatia amara. In certain embodiments, the formulation includes a composition of attenuations of Arnica montana, Calendula officinalis, Chamomilla, Echinacea angustifolia, and Ignatia amara. In certain embodiments, the formulation includes a composition of attenuations of Arnica montana, Calendula officinalis, Chamomilla, Echinacea purpurea, and Ignatia amara.

In certain embodiments, the formulation includes a composition of attenuations of Arnica montana, Calendula officinalis, Chamomilla, Echinacea angustifolia, Ignatia amara, and Hepar sulphuris calcareum. In certain embodiments, the formulation includes a composition of attenuations of Arnica montana, Calendula officinalis, Chamomilla, Echinacea purpurea, Ignatia amara, and Hepar sulphuris calcareum. In certain embodiments, the formulation includes attenuations of Arnica montana, Calendula officinalis, Chamomilla, Echinacea angustifolia, Ignatia amara, Hepar sulphuris calcareum, and Hypericum. In certain embodiments, the formulation includes a composition of attenuations of Arnica montana, Calendula officinalis, Chamomilla, Echinacea purpurea, Ignatia amara, Hepar sulphuris calcareum, and Hypericum. In certain embodiments, the formulation includes a composition of attenuations of Arnica montana, Calendula officinalis, Chamomilla, Echinacea angustifolia, Ignatia amara, Hepar sulphuris calcareum, Hypericum, and Mercurius vivus. In certain embodiments, the formulation includes a composition of attenuations of Arnica montana, Calendula officinalis, Chamomilla, Echinacea purpurea, Ignatia amara, Hepar sulphuris calcareum, Hypericum, and Mercurius vivus. In certain embodiments, the formulation includes a composition of attenuations of Arnica montana, Calendula officinalis, Chamomilla, Echinacea angustifolia, Ignatia amara, Hepar sulphuris calcareum, Hypericum, Mercurius vivus, and Ruta graveolens. In certain embodiments, the formulation includes a composition of attenuations of Arnica montana, Calendula officinalis, Chamomilla, Echinacea purpurea, Ignatia amara, Hepar sulphuris calcareum, Hypericum, Mercurius vivus, and Ruta graveolens. In certain embodiments, the formulation includes a composition of attenuations of Arnica montana, Calendula officinalis, Chamomilla, Echinacea angustifolia, Ignatia amara, Hepar sulphuris calcareum, Hypericum, Mercurius vivus, Ruta graveolens, and Plantago major. In certain embodiments, the formulation includes a composition of attenuations of Arnica montana, Calendula officinalis, Chamomilla, Echinacea purpurea, Ignatia amara, Hepar sulphuris calcareum, Hypericum, Mercurius vivus, Ruta graveolens, and Plantago major. In certain embodiments, the formulation includes a composition of attenuations of Arnica montana, Calendula officinalis, Chamomilla, Echinacea angustifolia, Ignatia amara, Hepar sulphuris calcareum, Hypericum, Mercurius vivus, Ruta graveolens, Plantago major, and Ledum palustre. In certain embodiments, the formulation includes a composition of attenuations of Arnica montana, Calendula officinalis, Chamomilla, Echinacea purpurea, Ignatia amara, Hepar sulphuris calcareum, Hypericum, Mercurius vivus, Ruta graveolens, Plantago major, and Ledum palustre. In certain embodiments, the formulation includes a composition of attenuations of Arnica montana, Calendula officinalis, Chamomilla, Echinacea angustifolia, Ignatia amara, Hepar sulphuris calcareum, Hypericum, Mercurius vivus, Ruta graveolens, Plantago major, Ledum palustre, and Silicea. In certain embodiments, the formulation includes a composition of attenuations of Arnica montana, Calendula officinalis, Chamomilla, Echinacea purpurea, Ignatia amara, Hepar sulphuris calcareum, Hypericum, Mercurius vivus, Ruta graveolens, Plantago major, Ledum palustre, and Silicea.

In some embodiments, the formulation includes a composition of attenuations of Arnica montana, Calendula officinalis, Chamomilla, Echinacea angustifolia, Ignatia amara and Apis mellifica. In other embodiments, the formulation includes a composition of Arnica montana, Calendula officinalis, Chamomilla, Echinacea purpurea, Ignatia amara, and Apis mellifica. In some embodiments, the formulation includes a composition of attenuations of Arnica montana, Calendula officinalis, Chamomilla, Echinacea angustifolia, Ignatia amara, Apis mellifica, and Rhus toxicodendron. In other embodiments, the formulation includes a composition of attenuations of Arnica montana, Calendula officinalis, Chamomilla, Echinacea purpurea, Ignatia amara, Apis mellifica, and Rhus toxicodendron. In some embodiments, the formulation includes a composition of attenuations of Arnica montana, Calendula officinalis, Chamomilla, Echinacea angustifolia, Ignatia amara, Apis mellifica, Rhus toxicodendron, and Symphytum officinale. In other embodiments, the formulation includes a composition of attenuations of Arnica montana, Calendula officinalis, Chamomilla, Echinacea purpurea, Ignatia amara, Apis mellifica, Rhus toxicodendron, and Symphytum officinale. Any of the compositions and formulations thereof described herein may additionally include Azadirachta indica.

In certain embodiments, a formulation of a composition described herein includes attenuations of Hepar sulphuris calcareum, Calendula officinalis, Chamomilla, Aconitum napellus, Echinacea angustifolia, Echinacea purpurea, Ruta graveolens, Arnica montana, Mercurius vivus, Hypericum, Gelsemium, Ignatia amara. In certain embodiments, a formulation includes additional ingredients for maintenance of oral care, wherein maintenance of oral care includes general oral maintenance, reduction of flora in the mouth, reduction of pain in the oral cavity, reduction of inflammation in the oral cavity, reduction of edema in the oral cavity, reduction of bruising in the oral cavity, treatment of trismus, recovery after dental surgeries, pre-treatment prior to surgery pertaining to the oral cavity, amelioration of anxiety associated with dental care, or preventative measures for maladies of the oral cavity, such as cavities, tooth decay, bone loss, or periodontal pocket formation. The formulation can be a spray or drops and the spray or drops can be part of a kit. In certain embodiments, the kit includes a composition in a gel or spray formulation which includes attenuations of Hepar sulphuris calcareum, Calendula officinalis, Chamomilla, Aconitum napellus, Echinacea angustifolia, Echinacea purpurea, Ruta graveolens, Arnica montana, Mercurius vivus, Hypericum, Gelsemium, Ignatia amara and optionally additional ingredients. In certain embodiments, a composition in a rinse formulation includes attenuations of Calendula officinalis, Echinacea purpurea, Plantago major, Azadirachta indica and optionally additional ingredients. In certain embodiments, the kit includes the rinse described herein.

The present disclosure presents a method for maintaining oral health, wherein the maintaining of oral health includes general oral maintenance, reduction of flora in the mouth, reduction of pain in the oral cavity, reduction of inflammation in the oral cavity, reduction of edema in the oral cavity, reduction of bruising in the oral cavity, treatment of post-surgical trismus, trismus, recovery after dental surgeries, pre-treatment prior to surgery pertaining to the oral cavity and face, amelioration of anxiety associated with dental care, or preventative measures for maladies of the oral cavity such as cavities, tooth decay, bone loss or periodontal pocket formation. The method may include a composition in a formulation for maintenance of oral care, wherein the composition includes attenuations of Hepar sulphuris calcareum, Calendula officinalis, Chamomilla, Aconitum napellus, Echinacea angustifolia, Echinacea purpurea, Ruta graveolens, Arnica montana, Mercurius vivus, Hypericum, Gelsemium, Ignatia amara, and optionally additional ingredients. In certain embodiments, the method includes a formulation for maintenance of oral care which includes attenuations of Arnica montana, Calendula officinalis, Chamomilla, Echinacea angustifolia, and Ignatia amara, or combinations thereof. In certain embodiments, the method includes administering or applying a composition in a formulation for maintenance of oral care which includes attenuations of Arnica montana, Calendula officinalis, Chamomilla, Echinacea purpurea, and Ignatia amara.

The present disclosure presents a method of shortening the time to maximum pain, inflammation, and/or trismus such that the post-surgical time to complete healing is shortened from 5 to 80% as compared to post-surgical time to healing with other formulations. For example, post-surgical healing time is reduced by 30-50%. In certain embodiments, post-surgical healing time is reduced within a range of about 0% to about 10%, about 10% to about 20%, about 20% to about 30%, about 30% to about 40%, about 40% to about 50%, about 50% to about 60%, about 60% to about 70%, about 70% to about 80%, about 80% to about 90%, and about 90% to about 100%.

The present disclosure presents a method of promoting healing at a dental site.

The present disclosure presents a method of reducing the number of post-operative analgesics needed, in particular the number of post-operative opioids needed. In certain embodiments, when used in combination with other embodiments described herein, the dosage of narcotics necessary for relief of post-surgical pain is reduced. In certain embodiments, the narcotics need for alleviation of pain is 30-50% less when the formulation is used to treat pain as compared to other medications.

### DETAILED DESCRIPTION OF THE DISCLOSURE

Pain management is an important aspect of care associated with oral and maxillofacial surgery. Patients undergoing full mouth rehabilitation with dental implants followed by an opioid-sparing postsurgical pain-management treatment with liposomal bupivacaine 266 mg, reported significantly less postsurgical pain despite reduced opioid use as compared to controls (See, Iero et al., Int J Oral Maxillofac Implants. 2018 September/October; 33(5):1155-1164. doi: 10.11607/jomi.5938, which is hereby incorporated by reference in its entirety). Different techniques can be implemented to reduce the number of strong opioids prescribed in oral and maxillofacial surgery practice. Certain embodiments, methods and compositions of the present disclosure have been shown to reduce pre-operative and post-operative pain and/or anxiety.

Due to the importance of oral health and hygiene to overall health, international guidelines have been published for minimizing the impact of oral disease; reducing mortality from oral and craniofacial diseases; promoting sustainable policies for oral health systems in the United States, including private dental practices and public clinics; developing accessible cost-effective health care systems; and reducing the disparities in oral health among individuals of different socio-economic backgrounds (See, Hobdell. Global goals for oral health 2020. International Dental Journal (2003) 53, 285-288, which is hereby incorporated by reference in its entirety).

Medical treatment of poor oral health frequently involves dental procedures (e.g. surgery) with side effects including craniofacial swelling, edemas, pain, bruising, trismus, and inflammation. Corticosteroids and analgesics, such as nonsteroidal anti-inflammatory drugs (NSAIDs), are the current standard of care for the management of the side effects associated with such procedures (See, Osunde et al. Afr Health Sci. 2011 September; 11(3): 530-537, which is hereby incorporated herein by reference in its entirety). However, evidence from previous studies suggests that no single treatment effectively reduces the aforementioned side effects without causing additional undesirable side effects. For example, presently available types of analgesics used to reduce swelling, pain, and inflammation have been shown to also cause negative reactions (See, Pozzi et al., Ann Stomatol (Roma). 2011 July-December; 2(3-4 Suppl): 3-24, which is incorporated herein by reference in its entirety). In particular, many patients have allergic reactions to NSAIDs, such as aspirin, celecoxib, diclofenac, diflunisal, etodolac, ibuprofen, indomethacin, ketoprofen, ketorolac, nabumetone, naproxen, oxaprozin, piroxicam, and salsalate.

Present treatments also fail to address negative side effects of oral and maxillofacial surgery not necessarily caused by poor oral health. Wisdom tooth extraction, also referred to as impacted lower third molar extraction, is one of the most common procedures performed in oral and maxillofacial surgeons' offices. Post-operative side effects following wisdom tooth extraction typically include pain, trismus and facial swelling that reach peak levels 3-4 days following the surgery (See, Goldberg, et al. J Am Dent Assoc. 1985; 111:277, which is hereby incorporated by reference in its entirety). These sequelae can lengthen a patient's post-operative recovery time and interfere with his/her ability to resume normal daily activities (See, Osborn, et al. L J Oral Maxillofac Surg. 1985; 43:767 and White et al. J OMS, 2003; Issue 5, pp 535-544, each of which is hereby incorporated by reference in its entirety). While intra- and post-operative administration of corticosteroids is used to reduce inflammation, the overall impact on morbidity was observed to be highly variably (See, Hirschman et al. Clin Exp Dermatol 11:27, 1986; Seymour et al. Int J Oral Surg 13:457, 1984; Bystedt et al. Swed Dent J 9: 65, 1985; Edilby et al. [abstract]. J Dent Res 61:556, 1982, each of which is hereby incorporated by reference in its entirety).

The current dental model for pain management has been in place since the 1980s and includes use of opioids, NSAIDs, acetaminophen, and adjuncts, such as long-acting local anesthesia (See, Malmberg et al. Science. 1992; 257:1276-1279 and Stein, N Engl J Med. 1995; 332:1685-1690, each of which is hereby incorporated by reference in its entirety). An American Dental Association survey from 2011 showed that although a majority (74%) of oral and maxillofacial surgeons prefer use of ibuprofen for pain management following wisdom tooth extraction, 85% also prescribed an opioid analgesic post-surgery (most commonly hydrocodone or oxycodone) (See, Denisco et al. J Am Dent Assoc. 2011; 142:800-10, which is hereby incorporated by reference in its entirety). In fact, patients from 16 to 25 years of age have been observed to be first exposed to opioids through prescriptions from dental clinicians (See, Schroeder et al. JAMA Internal Medicine; doi: 10.1001/jamainternmed.2018.5419; Tatch, Walter; “Opioid Prescribing Can Be Reduced in Oral and Maxillofacial Surgery Practice,” Journal of Oral and Maxillofacial Surgery; available online 19 Mar. 2019; https://doi.org/10.1016/j.joms.2019.03.009; each of which is hereby incorporated by reference in its entirety). From 2010-2015, the rate of opioid prescriptions per 1,000 dental patients increased from 130.58 to 147.44 prescriptions (See, Gupta et al. J Am Dent Assoc. 2018 April; 149(4):237-245.e6. doi: 10.1016/j.adaj.2018.01.005, which is hereby incorporated by reference in its entirety).

Results of studies on the effects of steroids on post-operative swelling and pain following oral surgery have been largely discouraging. For instance, steroids alone have not been observed to have a clinically significant analgesic effects (See, Miles et al. J Oral Maxillofac Surg 51: 987, 1993 and Dionne et al. J Oral Maxillofac Surg 61: 997, 2003, each of which is hereby incorporated by reference in its entirety). Submucosal steroid injection following surgical removal of third molars was confirmed to not provide significant reduction in post-operative pain in Grossi et al. (See, Grossi et al. J Oral Maxillofacial Surg 2007, 65: 2218-2226, which is hereby incorporated by reference in its entirety). In fact, steroids were observed to potentially enhance rather than reduce pain by suppressing beta-endorphin levels in the patient.

For example, Hargreaves et al. teaches that after the removal of impacted molars, “ . . . all doses of dexamethasone suppressed the postoperative increase in circulating levels of immunoreactive β-endorphin. Patients administered 0.1 mg dexamethasone reported greater levels of pain, compared with those given placebo, from 60 through 120 minutes after surgery. Post-operative pain for the patients administered the 0.32 mg and 1.0 mg doses did not differ from that for the placebo group. The increased pain after suppression of β-endorphin release by the low dose of dexamethasone suggests that pituitary secretion of immunoreactive β-endorphin alleviates postoperative pain under these conditions.” (See, Hargreaves et al. Clin Pharmacol Ther 42:601, 1987, which is hereby incorporated by reference in its entirety).

As efficacy of therapeutic combinations can be highly variable, a practitioner must take great care to avoid those with little-to-no efficacy, as well as combinations with contradictory outcomes. Foremost, a practitioner must never prescribe combinations that are potentially lethal.

Current therapies also often lack patient compliance, highlighting a need for more patient-friendly treatment options.

The FDA Compliance Policy Guide 7132.15 “Conditions Under Which Homeopathic Drugs May Be Marketed,” and the Homeopathic Pharmacopeia of the United States (HPUS, http://www.hpus.com/), as part of its regulation of homeopathics, publishes standards and monographs of homeopathic substances. HPUS encompasses drug monographs for over 1300 official substances. Each monograph lists identifying data for the drug, as well as specific manufacturing standards. HPUS is hereby incorporated by reference herein in its entirety.

### I. Compositions

The substances used to prepare tinctures and the attenuations that are components of the compositions described herein were obtained from plants, minerals, or animal substances. In certain embodiments, the substances can be obtained from pharmacies or online sources. Tinctures are generally prepared from fresh botanical or zoological material. According to the HPUS, water contained in the fresh material is considered a solvent and is not calculated as part of the medicinal raw material, and the moisture content of the fresh material is considered part of the diluent. Therefore, the calculated dry weight of the medicinal raw material, rather than the total weight of the fresh material, is taken as the starting point from which to prepare a tincture. Accordingly, the anhydrous form of acids, bases, and other chemicals is used as the starting point to prepare a tincture.

Tinctures are diluted to the final potency, attenuation, or concentration using a decimal dilution or decimal scale as described in the HPUS. The final potency of each remedy can be denoted by “X” or “D”, specifying the decimal dilution scale. There are other applicable scales that some practitioners use, such as the centesimal and quintamillesimal scales.

According to the decimal scale, each successive dilution will dilute the original volume of the substance by ten-fold. In the decimal scale, the original quantity of medicinal raw material is divided progressively by ten so that the first decimal attenuation (1X) is prepared using one (1) part of medicinal raw material in a total of ten (10) parts of finished first decimal attenuation. For example, a 1X dilution has an ingredient to solvent ratio of 1:10, a 2X dilution has an ingredient to solvent ratio of 1:100, a 3X dilution has an ingredient to solvent ratio of 1:1000, a 4X dilution has an ingredient to solvent ratio of 1:10000, a 5X dilution has an ingredient to solvent ratio of 1:100000, a 6X dilution has an ingredient to solvent ratio of 1:1000000, a 7X dilution has an ingredient to solvent ratio of 1:10⁷, a 8X dilution has an ingredient to solvent ratio of 1:10⁸, a 9X dilution has an ingredient to solvent ratio of 1:10⁹, a 10X dilution has an ingredient to solvent ratio of 1:10¹⁰, a 11X dilution has an ingredient to solvent ratio of 1:10¹¹, a 12X dilution has an ingredient to solvent ratio of 1:10¹², a 13X dilution has an ingredient to solvent ratio of 1:10¹³, a 14X dilution has an ingredient to solvent ratio of 1:10¹⁴, a 15X dilution has an ingredient to solvent ratio of 1:10¹⁵, a 16X dilution has an ingredient to solvent ratio of 1:10¹⁶, a 17X dilution has an ingredient to solvent ratio of 1:10¹⁷, a 18X dilution has an ingredient to solvent ratio of 1:10¹⁸, a 19X dilution has an ingredient to solvent ratio of 1:10¹⁹, a 20X dilution has an ingredient to solvent ratio of 1:10²⁰, a 21X dilution has an ingredient to solvent ratio of 1:10²¹, a 22X dilution has an ingredient to solvent ratio of 1:10²², a 23X dilution has an ingredient to solvent ratio of 1:10²³, a 24X dilution has an ingredient to solvent ratio of 1:10²⁴, a 25X dilution has an ingredient to solvent ratio of 1:10²⁵, a 26X dilution has an ingredient to solvent ratio of 1:10²⁶, a 27X dilution has an ingredient to solvent ratio of 1:10²⁷, a 28X dilution has an ingredient to solvent ratio of 1:10²⁸, a 29X dilution has an ingredient to solvent ratio of 1:10²⁹, a 30X dilution has an ingredient to solvent ratio of 1:10³⁰, a 31X dilution has an ingredient to solvent ratio of 1:10³¹, a 32X dilution has an ingredient to solvent ratio of 1:10³², a 33X dilution has an ingredient to solvent ratio of 1:10³³, a 34X dilution has an ingredient to solvent ratio of 1:10³⁴, a 35X dilution has an ingredient to solvent ratio of 1:10³⁵, and so on as shown in Table 1. Dilutions are also referred to herein as potency levels.

TABLE 1
Examples of Decimal Dilutions

| Potency | Dilution | Concentration |
|---|---|---|
| 1X or 1D | 1:10 | 10⁻¹ |
| 2X or 2D | 1:100 | 10⁻² |
| 3X or 3D | 1:1000 | 10⁻³ |
| 4X or 4D | 1:10000 | 10⁻⁴ |
| 5X or 5D | 1:100000 | 10⁻⁵ |
| 6X or 6D | 1:1000000 | 10⁻⁶ |
| 7X or 7D | 1:10000000 | 10⁻⁷ |
| 8X or 8D | 1:100000000 | 10⁻⁸ |
| 10X or 10D | 1:10000000000 | 10⁻¹⁰ |
| 12X or 12D | 1:10¹² | 10⁻¹² |
| 18X or 18D | 1:10¹⁸ | 10⁻¹⁸ |
| 30X or 30D | 1:10³⁰ | 10⁻³⁰ |

Substances can be present in any amount that is safe and effective. The substances can be present in dilutions from 1X to 40X. The substances can be present in dilutions of 1X, 2X, 3X, 4X, 5X, 6X, 7X, 8X, 9X, 10X, 11X, 12X, 13X, 14X, 15X, 16X, 17X, 18X, 19X, 20X, 21X, 22X, 23X, 24X, 25X, 26X, 27X, 28X, 29X, 30X, 31X, 32X, 33X, 34X, 35X, 36X, 37X, 38X, 39X, or 40X.

The present disclosure presents compositions which include tinctures of one or more ingredients. In certain embodiments, a tincture can be a mother tincture or mother concentrate (i.e. 1X attenuation) on an ingredient. In certain embodiments, a tincture can be a diluted attenuation (e.g. 2X or 4X attenuation) of a mother tincture or mother concentrate of an ingredient. While plants are the base substances for most tinctures, some are made from mineral, animal, or other substances.

The extraction process may include the steps of comminution, maceration, percolation, decoction, and fermentation according to the HPUS. In some instances, a part of the plant or source material is isolated or removed and crushed. A mother tincture typically has a 1X potency (1×10⁻¹ dilution) according to the decimal scale. The mother tincture can be further diluted with a solvent and/or succussed, i.e., vigorously shaken.

The tincture can be prepared by exposing a part, more than one part and/or the entirety of the raw ingredient in a solvent. Suitable solvents may include alcohol, water, water-alcohol mixtures, glycerine or isotonic sodium chloride solutions. In some embodiments of a tincture, the alcohol is ethanol denoted as Alcohol 96%, which is also referred to as a strong alcohol, with a chemical formula of C₂H₅OH and a molecular weight (mw) of 46.07 g/mol. Alternatively, the alcohol may be chosen from anhydrous alcohol, which is also referred to as dehydrated alcohol and absolute alcohol; Alcohol, 35% (v/v); Alcohol, 45% (v/v); Alcohol, 50% (v/v); Alcohol, 55% (v/v); Alcohol, 60% (v/v); Alcohol, 65% (v/v); Alcohol, 70% (v/v); Alcohol, 80% (v/v); or Alcohol, 90% (v/v) as defined in the United States Pharmacopeia (USP). In yet certain embodiments, neutralized alcohol aldehyde-free alcohol (USP Reagent) is used as the solvent.

### Classifications of Triturations

### Class F

Attenuations of solid medicinal raw materials are prepared by trituration of the medicinal raw material with lactose monohydrate as described in the HPUS. In calculating the ratio of raw material to diluent, the anhydrous form of the raw material is the basis for calculation. The trituration process must be continued for a sufficient time period to ensure that a homogenous mass is prepared.

Generally, lactose monohydrate is the vehicle for the preparation of triturations. Lactose monohydrate (C₁₂H₂₂O₁₁, mw 342.30 g/mol) must meet the tests for identity and purity in the United States Pharmacopoeia (USP). For decimal attenuations, one (1) part of the medicinal raw material is triturated with nine (9) parts of lactose monohydrate resulting in a 1X trituration.

### Class G

Attenuations of insoluble liquid medicinal raw materials are prepared by trituration of the medicinal raw material with lactose monohydrate as described in the HPUS. In calculating the ratio of raw material to diluent, the anhydrous form of the raw material is the basis for calculation. A mortar and pestle may be used for small amounts, as defined in the art; and a mechanical triturator may be used for large amounts, as defined in the art.

For decimal attenuations, one (1) part of the insoluble liquid raw material is triturated with nine (9) parts of lactose monohydrate. The result is the 1X trituration.

### Classifications of Tinctures

The substances herein that may be included in the compositions described herein are described according to the classifications defined in the HPUS, which is incorporated by reference in its entirety. The class of a substance defines how a tincture is prepared from the medicinal raw material.

The use of the word “tincture” implies the product has the concentration or ratio of starting material to finished tincture shown in Table 2.

TABLE 2
Tincture concentrations

| Source | Class | Concentration |
|---|---|---|
| Botanical Tinctures | Class C | 1:10 or 10% (1X) |
| Sarcodes | Class E | 1:10 or 10% (1X) |
| | (Method I) | |
| Sarcodes | Class E | 1:20 or 5% |
| | (Method II) | |
| Succus Or Non-Alcoholic Extracts | Class O | 1:2 or 50% |
| Fresh Botanical Raw Materials | Class M | 1:2 or 50% |
| Fresh Botanical Raw Materials | Class N | 1:3 or 33.% |

### Class C

Class C tinctures are prepared from fresh succulent plants and plant parts containing water, as described in the HPUS. For preparation of a Class C tincture, the plant moisture is calculated as part of the total preparation. The calculated dry weight of the medicinal raw material remaining after evaporation is taken as the unit of strength from which to calculate the strength of the tincture. The tincture is prepared in the proportion of one (1) part of equivalent dried medicinal raw material in a total of ten (10) parts of tincture using alcohol of the strength specified in Table 3. The tincture is equal in concentration to the first decimal attenuation (1X).

### Class E

Class E tinctures are prepared from entire animals or zoological materials, such as parts of animals, as described in the HPUS. The medicinal raw material is extracted from the zoological materials by maceration as described in the HPUS, which is hereby incorporated by reference in its entirety.

In some embodiments, tinctures are prepared using Method I in which the proportion of one (1) part of the medicinal raw material in a total of ten (10) parts of finished tincture using the strength of alcohol specified in Table 3. The resulting tincture 1:10 or 10% is the strongest liquid preparation made directly from the medicinal raw material and is the first decimal attenuation (1X).

In other embodiments, tinctures are prepared using Method II in which the proportion of one (1) part of the medicinal raw material in a total of twenty (20) parts of tincture using the strength of alcohol specified in Table 3. The resulting tincture is 1:20 or 5% and is the strongest liquid preparation made directly from the medicinal raw material. The 2X attenuation is prepared by using two (2) parts of this 1:20 tincture with eight (8) parts of diluent, such as alcohol or water.

If the animals serving as the basis for the Class E tincture was incapacitated using alcohol, the quantity and strength of the alcohol used is considered when calculating the amount of alcohol to be added when preparing the tincture. Chemical substances should be avoided for incapacitation.

### Class M

Class M tinctures generally are prepared from fresh botanical raw materials by maceration, as described in the HPUS. Alternatively, extraction of medicinal raw materials is performed by other methods described herein. Class M tinctures are prepared in the ratio of one (1) part of the fresh botanical raw material's moisture content, in two (2) parts of completed solution. To calculate the amount of alcohol needed, use the following equation:

A=(W×M)/100; wherein A=weight of alcohol to be added; W=weight of the fresh botanical raw material; and M=loss of plant moisture in percent.

For decimal attenuations, two (2) parts of the Class M tincture is added to eight (8) parts of the diluent, such as alcohol or water, and the mixture is succussed. The result is the 1X attenuation.

### Class N

Class N tinctures generally are prepared by maceration from fresh botanical raw materials, as described in the HPUS. Alternatively, extraction of medicinal raw materials is performed by other methods of preparation. Class N tinctures are prepared in the ratio of one (1) part of the fresh botanical raw material's moisture content, in three (3) parts of completed solution. To calculate the amount of alcohol needed, use the following equation:

A=(2W×M)/100; wherein A=weight of alcohol to be added; W=weight of the fresh botanical raw material; and M=loss of plant moisture in percent.

The resulting tincture (33.3%) is the strongest liquid preparation made directly from the medicinal raw material. For decimal attenuations, three (3) parts of Class N tincture is added to seven (7) parts of diluent, such as alcohol or water, and the mixture is succussed. The result is the 1X attenuation.

### Class H

Class H tinctures are triturations of insoluble medicinal raw material converted into liquid attenuations, as described in the HPUS. Class H tinctures are prepared by attenuating one (1) part of the lowest soluble trituration of the medicinal raw material with sufficient diluent to produce the next attenuation. All chemical raw materials that are insoluble or only partially soluble in the provided vehicle must only be prepared as triturations through two (2) decimal steps below the desired liquid attenuation. The trituration may then be converted to liquid according to the following procedure(s):
1. Dissolve one (I) part of the lowest soluble trituration available in a sufficient amount of purified water to make a total of ten (10) parts.
2. Succus the mixture to create the intermediate attenuation.
3. Add nine (9) parts of diluent to one (1) part of the intermediate attenuation.
4. Succus the mixture of step 3, which results the desired liquid attenuation.

The purified water should meet the tests for purity described in the United States Pharmacopoeia (USP). Glycerin (Glycerinum, Glycerol) is a Polyhydric Alcohol which contains 95% C₃H₅(OH)₃.

### Substances and Ingredients

Table 3 lists examples of substances or ingredients which may be combined to result in the compositions of the present disclosure. Values and information in Table 3 are from the HPUS, which is hereby incorporated by reference in its entirety. The column labeled “Liquid class” provides the appropriate class reference in the General Pharmacy section of the HPUS under which the product should be prepared in liquid form, if such form is possible. The column labeled “percent alcohol” provides the finished product alcohol percentage or strength of the alcohol for the liquid preparation of the tincture. The column labeled “OTC” provides the attenuation at or above which a drug may be offered for sale without a prescription or “over the counter” for the purpose of a systemic effect. The column labeled “External use” provides the attenuation at, or above which, a drug may be offered for sale without a prescription for “external or topical use only”; this should take into consideration both the intended therapeutic effect and the method of administration. Topical dosage forms are defined in the Guidelines for Manufacturing Homeopathic Medicines in the HPUS, which is hereby incorporated by reference in its entirety. The column labeled “Rx form” provides the attenuation at or above which a drug may only be offered for sale with a prescription (or Rx) for the purpose of a systemic effect. The Rx-only restriction would be valid up to the OTC attenuation noted for the ingredient in Table 3. Attenuations below the Rx form attenuation may not be offered for sale to the public. The term “tincture” is used when the drug is used in the tincture form from Class C or other appropriate classes. “NA” indicates the column is not applicable for the particular drug. For example, OTC=1X, Rx=N/A, HPN=N/A indicates that all attenuations starting from 1X are available for sales without a prescription. Therefore, the Rx and HPN potency limits are not applicable, and “NA” in the External use column means that the HPUS does not have data showing the safe external use of the substance to result in a systemic effect.

TABLE 3
Substances for therapeutic compositions

| | | Percent | | | | | Dry residue |
|---|---|---|---|---|---|---|---|
| | Liquid | alcohol | | External | | Source of the | weight in |
| Substance | class | [v/v ± 15%] | OTC | use | Rx form | tincture | tincture [% w/w] |
| Aconitum napellus | C or M | 45 | 3X | 3X | Tincture | Whole plant | not less than 1.5 |
| Allium sativum | C or N | 55 | Tincture | NA | NA | Mature bulb | not less than 4.0 |
| Apis mellifica | E | 65 | Tincture | Tincture | NA | Live bees | not less than 0.25 |
| Argentum nitricum | A | 0 | 5X | 4X | 2X | Soluble crystals | NA |
| Aristolochia clematitis | C | 45 | 6X | NA | NA | Root | NA |
| Arnica montana | C or N | 45 | 3X | Tincture | Tincture | Root | not less than 1.0 |
| Arsenicum album | B | 0 | 6X | 6X | 3X | NA | not less than 99.5, |
| | | | | | | | not more than 100.5 |
| Azadirachta indica | C | 65 | Tincture | NA | NA | Whole bark | NA |
| (also referred to as | | | | | | | |
| Melia Azadirachta) | | | | | | | |
| Berberis vulgaris | C | 55 | 3X | 2X | 2X | Bark of the root | not less than 0.7 |
| Calcarea carbônica | H | | 1 | NA | NA | Oyster shell | not less than 90 |
| Calendula officinalis | C or N | 45 | Tincture | Tincture | NA | Flowering tops | not less than 1.0 |
| Camellia sinensis | C | 65 | Tincture | NA | NA | Leaves | NA |
| Cantharis | E | 65 | 3X | 3X | 2X | Insect | not less than 0.5 and |
| | | | | | | | not more than 2.6 |
| Capsicum annuum | C | 90 | 3X | Tincture | Tincture | Dried ripe fruit | not less than 0.7 |
| Carbolicum acidum | B | 70 | 6X | 2X | 3X | Crystal | NA |
| Causticum | A | 55 | 3X | NA | 1X | Recently burnt | not more than 0.01 |
| | | | | | | lime | |
| Chamomilla | C | 45 | Tincture | Tincture | NA | Whole flowering | not less than 1.2 |
| | | | | | | plant | |
| Cinnamomum verum | C | 65 | Tincture | NA | NA | Inner bark | not less than 1.0 |
| Cinnamomum | C | 65 | Tincture | NA | NA | Inner bark | not less than 1.0 |
| zeylanicum Nees | | | | | | | |
| Citrullus colocynthis | C | 65 | 3X | NA | Tincture | Pulp of the fruit, | not less than 1.4 |
| | | | | | | rejecting the | |
| | | | | | | seeds | |
| Copaiva Officinalis | C | 90 | Tincture | NA | NA | Oleoresin | not less than 2.0 |
| Croton lechleri | | | | | | Tree sap | |
| Echinacea augustifolia | C or N | 55 | Tincture | Tincture | NA | Whole plant | not less than 0.7 |
| Echinacea purpurea | C | 55 | Tincture | Tincture | NA | Whole plant | NA |
| Fagopyrum esculentum | C | 65 | Tincture | NA | NA | Whole plant | not less than 1.0 |
| Gelsemium | C or N | 65 | 3X | NA | Tincture | Root | not less than 0.5 |
| Graphites | H | | 1X | 1X | NA | NA | not less than 96.0, |
| | | | | | | | not more than 102.0 |
| Hepar sulphuris calcareum | H | | 1X | 1X | NA | Amorphous | NA |
| | | | | | | powder | |
| Hydrastis canadensis | C | | 5X | 4X | 3X | Rhizome and | NA |
| | | | | | | roots | |
| Hypericum | C or N | 65 | 3X | Tincture | Tincture | Whole plant | not less than 1.3 |
| Ignatia amara | C | 65 | 3X | NA | Tincture | Dried seeds | not less than 1.1 |
| Juglans regia | C | 65 | 5X | 4X | 3X | Leaves and of | not less than 2.0 |
| | | | | | | rind of the green, | |
| | | | | | | unripe fruit, in | |
| | | | | | | equal parts | |
| Lac caninum | B | 0 | 3X | NA | 2X | mammary glands | NA |
| | | | | | | of a lactating dog | |
| Lachesis mutus | B | 0 | 8X | 8X | 6X | Snake Venom | NA |
| Ledum palustre | C | 65 | 4X | 2X | 2X | Leafy twigs | not less than 1.0 |
| Mahonia aquifolium | C | 55 | 3X | 2X | 2X | Bark of the root | not less than 0.5 |
| Melissa officinalis | C | 65 | Tincture | Tincture | NA | Whole plant | NA |
| Mercurius vivus | H | NA | 6X | 6X | 3X | Silver-white | not less than 99.5 |
| | | | | | | metal | |
| Natrum Bicarbonicum | A | 0 | 1X | NA | NA | NA | NA |
| Okoubaka aubrevillei | C | 65 | 2X | NA | Tincture | Bark | not less than 0.4 |
| Origanum majorana | C | 65 | Tincture | Tincture | NA | Whole plant | not less than 1.0 |
| Paullinia cupana | C | 65 | Tincture | NA | NA | Paste made from | not less than 1.3 |
| | | | | | | the seeds | |
| Petroselinum crispum | C or N | 65 | Tincture | NA | NA | Whole plant | not less than 1.8 |
| Phosphorus | B | 95 | 4X | 4X | NA | NA | NA |
| Phytolacca decandra | C or N | 55 | 4X | 4X | 2X | Root | not less than 2.2, |
| | | | | | | | not more than 3.5 |
| Plantago major | C | 65 | Tincture | Tincture | NA | Whole plant | not less than 1.2 |
| Punica granatum | C | 65 | Tincture | NA | NA | Root bark | |
| Rhus toxicodendron | C | 65 | 3X | NA | T | Fresh leaves | not less than 1.2 |
| Ruta graveolens | C or N | 65 | 3X | Tincture | Tincture | Aerial parts | not less than 1.5 |
| Salvia officinalis | C or N | 55 | Tincture | Tincture | NA | Leaves | not less than 1.0 |
| Sambucus nigra | C | 45 | Tincture | Tincture | NA | Flowering tops | not less than 1.7 |
| Silicea | H | NA | 2X | 1X | 1X | Transparent | NA |
| | | | | | | crystals or while, | |
| | | | | | | amorphous | |
| | | | | | | powder | |
| Strontium carbinicum | H | NA | 6X | NA | 3X | NA | not less than 98.5, |
| | | | | | | | not more than 100.5 |
| Symphytum officinale | C | 45 | 6X | 6X | 3X | Root | not less than 1.5 |
| Syzygium aromaticum | C | 65 | Tincture | Tincture | NA | Flower buds | not less than 1.5 |
| Thiosinaminum | A | 95 | 3X | 1X | 1X | Oil of mustard | NA |
| Thuja occidentalis | C or N | 65 | Tincture | Tincture | NA | Leafy twigs | not less than 1.3 |
| Thymus serpyllum | C | 65 | Tincture | NA | NA | Whole plant | NA |
| Vitis Vinifera | C | 35 | 2X | NA | NA | Flowering | NA |
| | | | | | | clusters | |
| Xanthoxylum | C | 65 | Tincture | NA | NA | Bark | NA |
| (Zanthoxylum) | | | | | | | |

Dry residue is the mass remaining in percent weight by weight (%, w/w) after drying. Dry residue was measured by weighing about 5 g of the test substance in a flat-bottomed glass, where it was evaporated to dryness on a water bath. The substance was heated in an oven at 100-105° C. and dried for at least two hours, and then to constant mass at 100-105° C. After being allowed to cool in a desiccator, the remaining dry residue was weighed and calculated as the percentage of residue for 100 g of test material.

In some embodiments, the substance should be prepared in a solid form. For example, according to the HPUS, Hepar sulphuris calcareum and Silicea are classified as Class F, which is a classification of a substance that is in solid form as a medicinal raw material. Further, Mercurius vivus is classified as Class G, which is a classification of substances that are insoluble liquids as medicinal raw material. In some embodiments, the tincture of Mercurius vivus contains 10 mg/ml of the silver-white metal that is the medicinal raw material.

The monograph for Aconitum napellus in the HPUS notes a tincture of the substance should contain not less than 0.025% and not more than 0.075% of alkaloids, calculated as aconitine [C₃₄H₁₄₇NO₁₁; mw 646]. The monograph for Berberis vulgaris in the HPUS notes a tincture of the substance should contain not less than 0.18% and not more than 0.60% of alkaloids, calculated as berberine (C₂₀H₁₉NO₅; mw 353.4). The monograph for Plantago major in the HPUS notes a tincture contains not less than 0.02% and not more than 0.08% w/w of aucubin (C₁₅H₂₂O₉; mw 346.33). The monograph for Rhus toxicodendron in the HPUS notes a tincture contains not less than 0.12% of tannins, expressed as pyrogallol (C₆H₆O₃; mw 126.1). The monograph for Hydrastis canadensis in the HPUS notes the starting material for preparation of a tincture should contain not less than 2.50% of hydrastine (C₂₁H₂₁NO₆, mw 383.40) and not less than 3.0% of berberine (C₂₀H₁₉NO₅, mw 353.37). Further, a tincture of Hydrastis canadensis should contain not less than 0.15% and not more than 0.25% of hydrastine (C₂₁H₂₁NO₆, mw 383.40), and not less than 0.27% and not more than 0.50% of berberine (C20H19NO5, mw 353.37). The monograph for Hepar sulphuris calcareum in the HPUS notes the starting material for preparation of a tincture should contain not less than 24.0% w/w and not more than 33.0% w/w of sulfur (S; atomic weight 32.0) as sulfide. The monograph for Juglans regia in the HPUS notes a tincture contains not less than 0.002% and not more than 0.008% w/w of total quinone derivatives, expressed as juglone (C₁₀H₆O₃; mw 171.2). The monograph for Gelsemium in the HPUS notes a tincture contains not less than 0.015% and not more than 0.080% of alkaloids, calculated as gelsemine (C₂₀H₂₂N₂O₂; mw 322.4). The monograph for Fagopyrum esculentum in the HPUS notes a tincture contains not less than 0.060% w/w of total flavonoids, expressed as rutin (C₂₇H₃₀O₁₆.3H₂O; mw 664.57). The monograph for Ignatia amara in the HPUS notes a tincture contains not less than 0.12% and not more than 0.17% of strychnine (C₂₁H₂₂N₂O₂; mw 334.4); not less than 0.18% and not more than 0.36% overall of strychnine and brucine; and not less than 55% of which is strychnine (C₂₁H₂₂N₂O₂; mw 334.4). The monograph for Paullinia cupana in the HPUS notes a tincture contains not less than 0.25% w/w of caffeine (C₈H₁₀N₄O₂; mw 194.2). The monograph for Calcarea carbonica in the HPUS notes a tincture contains not less than 90.0% w/w of calcium carbonate (CaCO3; m.w. 100.1). The monograph for Arsenicum album in the HPUS notes a tincture of the substance should contain not less than 99.5% and not more than 100.5% w/w of arsenic trioxide.

Arnica montana, which is included in the compositions described herein, contains phenolic and flavonoids compounds that lower expression levels of nitric oxide, TNF-alpha, and interleukins (II-1beta, II-6 and II-12) (See, Esenet al. J Oral Maxillofacial Surg. 1999; 57: 1201, which is hereby incorporated by reference in its entirety). Arnica montana also stimulates extracellular matrix gene expression in a macrophage cell line with a wound-healing phenotype (See, Nayyar et al. Br J Oral Maxillofacial Surg. 2006; 44:501, which is hereby incorporated by reference in its entirety). Arnica montana is a common remedy for trauma, especially post-surgery. Its use following plastic surgery, in particular, is widespread. A randomized, double-blind, placebo-controlled study investigating the effect of Arnica montana on bruising associated with face lift procedures showed that administration or application of Arnica montana perioperatively resulted in significantly less ecchymosis in patients (See, Brook et. al Arch Facial Plast Surg. 2006; 8(1): 54-59, which is hereby incorporated by reference in its entirety). Results of other studies on the efficacy of Arnica montana, however, are contradictory (See, Kaziro et al. Br J Oral Maxillofac Surg 1984; 2242-49, which is hereby incorporated by reference in its entirety). Arnica montana is thought to increase blood flow surrounding bruised tissue, thereby aiding in the absorption of escaped fluids by the body. This process reduces swelling and relieves pressure on nerve endings that may otherwise cause pain.

Acontium has been shown both in vitro and in vivo to have long-acting local anesthetic properties, likely through a dose-dependent mechanism involving reduced neuronal sodium currents (See, Shugars et al. J Oral Maxillofacial Surg 1996, 54: 1402-1408; Grossi et al. J Oral Maxillofacial Surg 2007, 65: 2218-2226, each of which is hereby incorporated by reference in its entirety).

One of the active alkaloids of Gelsemium is koumine, which has been found to have anti-allodynic and neuroprotective effects. It has been further recommended for use in diabetic neuropathy (See, Miles et al. J Oral Maxillofac Surg 51: 987, 1993; Dionne et al. J Oral Maxillofac Surg 61: 997, 2003, each of which is hereby incorporated by reference in its entirety). Further, Gelsemium was found to alleviate both neuropathic pain and sleep disturbances (See, Hargreaves et al. Clin Pharmacol Ther 42:601, 1987, which is hereby incorporated by reference in its entirety).

Alpha-Bisabolol, one of the essential oils isolated from Chamomilla, has been shown to decrease nervous excitability via blockade of voltage-dependent sodium channels (See, Alves et al. Neurosci Lett. 2010 Mar. 12; 472(1): 11-5. doi: 10.1016/j.neulet.2010.01.042. Epub 2010 Jan. 25, which is hereby incorporated by reference in its entirety). Chamomile was also found to selectively inhibit COX-2 (See, Ortiz et al. Biomed Pharmacother. 2016 March; 78:248-56. doi: 10.1016/j.biopha.2016.01.029. Epub 2016 Feb. 2, which is hereby incorporated by reference in its entirety). In a double-blinded, randomized controlled study, Chamomile was found to be a safe, well-tolerated, and effective treatment for women with moderate mastalgia (See, Saghafi et al. J Obstet Gynaecol. 2017 October 26:1-4. doi: 10.1080/01443615.2017.1322045, which is hereby incorporated by reference in its entirety). Calendula officinalis has been found beneficial for diabetic foot ulcers as well as venous leg ulcers (See, Buzzi et al. Ostomy Wound Manage. 2016 March; 62(3):8-24, which is hereby incorporated by reference in its entirety).

In certain embodiments, the compositions described herein include one or more active ingredients or inactive ingredients as taught in United States Patent Application No: 2009/0087501 A1, which is incorporate herein by reference in its entirety as it relates to active or inactive botanical ingredients. In certain embodiments, the active ingredients or inactive ingredients include one or more of: Origanum vulgare, Origanum onites, Origanum majorana, Origanum heracleoticum, Thymus vulgaris L, Thymus citriodorus, Thymus pulegioides, Thymus x herba-barona, Thymus serpyllum, Lavandula angustifolia/officinalis, Lavandula stoechas, Lavandula dentate, Lavandula x intermedia, Lavandula multifida, Salvia officinalis, Salvia divinorum, Salvia apiana, Melissa Officinalis, Cuminum cyminum, Petroselinum crispum, Calendula arvensis, Calendula maderensis, Calendula officinalis, Tagetes erecta, Tagetes minuta, Tagetes patula, Boswellia sacra, Boswellia frereana, Boswellia serrata, Boswellia papyrifera, Sambucus nigra, Sambucus melanocarpa, Sambucus racemosa, Copaifera langsdorfii, Curcuma longa, Allium sativu, Symphytum officinale, Punica granatum, Euterpe oleracea, Sophora flavescens, Rheum rhabarbarum, Rheum rhaponticum, Fagopyrum esculentum, Camellia sinensis, Coptis teeta, Hydrastis canadensis, Mahonia aquifolium, Phellodendron amurense, Berberis vulgaris, Xanthorhiza simplicissima, Lonicera ceprifoliu, Vaccinium macrocarpon, Cinnamomum zeylanicum Nees, Cinnamomum verum, Vitis Vinifera, Terminalia Billerica, Pinus Pinaster, Albizia Lebbek, Melia Azadirachta, Salvadora persica, Paullinia cupana, Piper betle, Syzygium aromaticum, Commiphora myrrha, Juglans regia, Scutellaria baicalensis, and Magnolia officinalis . . . and one or more of plants of the following species: Romains officinalis, Origanum vulgare L, Lavandula angustifolia/officinalis, Salvia officinalis, Melissa officinalis, Cuminum cyminum, Petroselinum crispum, Calendula officinalis, Boswellia sacra, Sambucus nigra, Copaifera langsdorfii, Curcuma longa, Allium sativu, Symphytum officinale, Punica granatum, Euterpe oleracea, Sophora flavescens, Rheum rhabarbarum, Fagopyrum esculentum, Camellia sinensis, Coptis teeta, Hydrastis canadensis, Mahonia aquifolium, Phellodendron amurense, Berberis vulgaris, Lonicera ceprifoliu, Cinnamomum zeylanicum Nees, Cinnamomum verum, Vitis Vinifera, Pinus Pinaster, Albizia Lebbek, Melia Azadirachta, Salvadora persica, Paullinia cupana, Piper betle, Syzygium aromaticum, Commiphora myrrha, Juglans regia, Scutellaria baicalensis, and Magnolia officinalis.

In certain embodiments, the compositions described herein include one or more active ingredients or inactive ingredients as taught in U.S. Pat. No. 7,083,779, which is hereby incorporated herein by reference in its entirety as it relates to active or inactive botanical ingredients. In certain embodiments, the active ingredients or inactive ingredients include one or more synergistic herbal formulations which can include an active fraction from Azadirachta indica designated as Fraction A and a fraction from Citrullus colocynthis designated as Fraction B, along with a Fraction C containing an antioxidant from Cucumis sativus extract.

In certain embodiments, the compositions described herein include active ingredients and inactive ingredients. In certain embodiments, an active ingredient is selected from Calendula officinalis 1X , Echinacea purpurea 1X, Plantago major 1X, and Azadirachta indica/Neem 1X. In certain embodiments, an active ingredient is present in the composition in 20% alcohol. Alternatively, the active ingredient is present in the composition in less than 20% alcohol. Alternatively, the active ingredient is present in the composition in greater than 20% alcohol.

### Extraction

Medicinal raw material is extracted primarily by the process of maceration as described in the HPUS, which is hereby incorporated by reference in its entirety. In other embodiments, a method is used selected from the following: percolation, decoction, fermentation, incubation, infusion, and succus using the described in the HPUS, which is hereby incorporated by reference in its entirety. Maceration may be used for the extraction of large quantities of botanical raw material having significant quantities of substances that would prevent the alcohol from permeating the mass as rapidly as is necessary for extraction. Percolation may be used for the preparation of dried botanical raw materials that have been reduced to the proper degree of fineness. Decoction may be used for extraction of medicinal raw material from fibrous botanical raw material, such as roots, barks, and woody substances. Fermentation, specifically lactic acid fermentation or ethanolic fermentation, may be used for extraction of medicinal raw material from botanical raw materials for tinctures with a minimal amount of alcohol or intended to be alcohol free. Incubation may be used for extraction of medicinal raw material from botanical raw materials needing extended time for extraction and in which a gentle elevation of temperature will create a better breakdown of complex sugar constituents into simpler saccharides, leading to a more complete extraction of medicinal properties. Infusion may be used for extraction of medicinal raw material from dried botanical raw materials containing large amounts of aromatic principles, such as relatively high concentrations of dehydrated aliphatic hydrocarbons.

### Drugs

In certain embodiments, compositions and treatments of the present disclosure for post-surgical pain include multiple classes of drugs, such as but not limited to opioids, non-opioid analgesics, long-acting local anesthetics, and other similar alternatives. In certain embodiments, the compositions described herein are administered or applied to a patient in combination with a drug.

### Opioids

In certain embodiments, the compositions described herein are administered or applied to a patient in combination with an opioid, which is a class of drugs that acts as opioid receptor agonists. Patients who regularly consume opioids for longer than a week are likely to develop drug dependency (See, Hirschman J V; Clin Exp Dermatol 11:27, 1986, which is hereby incorporated by reference in its entirety). After repeated administration of opioids, patients build a tolerance and require an increasingly higher doses to achieve adequate pain relief (See, Seymour et al. Int J Oral Surg 13:457, 1984; Bystedt et al. Swed Dent J 9: 65, 1985, each of which is hereby incorporated by reference in its entirety).

In certain embodiments, hydrocodone/acetaminophen pills (also referred to as NORCO® pills or VICODIN® pills) are administered to a patient prior to oral surgery. In some embodiments, hydrocodone/acetaminophen pills (also referred to as NORCO® pills or VICODIN® pills) are administered concurrently to a patient.

### Non-Opioid Analgesics

In certain embodiments, the compositions described herein are administered or applied to a patient in combination with at least one non-opioid analgesic. Non-opioid analgesics are a class of drugs divided into non-steroidal anti-inflammatory drugs (NSAIDS) and acetaminophen (APAP). NSAIDs reduce the synthesis of prostaglandins responsible for generating pain, fever, and inflammation through inhibition of COX-1 and COX-2. The mechanism of APAP is hypothesized to involve inhibition of prostaglandin synthesis within the central nervous system, and indirect activation of cannabinoid CB(1) receptors (See, Edilby et al. J Dent Res 61:556, 1982; Brook et al. Arch Facial Plast Surg. 2006; 8(1):54-59, each of which is hereby incorporated by reference in its entirety).

Both, NSAIDs and acetaminophen exhibit a “ceiling” effect where higher doses do not provide greater analgesia. The “ceiling” dose is 400 mg for ibuprofen, and 1000 mg for acetaminophen.

Some studies have shown that the combination of NSAIDs and APAP provide greater analgesia than opioids at conventional dosages (See, Kaziro et al. Br J Oral Maxillofac Surg 1984; 2242-49; Noroozi et al. OOO, 2009; 107:30; Kim et. al. OOO, 2006; 102:e4, each of which is hereby incorporated by reference in its entirety). In some embodiments, the NSAID is ibuprofen. In some embodiments, ibuprofen is administered to a patient prior to oral surgery and compositions described herein, also referred to as VEGA™ compositions, are administered post-operatively. In some embodiments, compositions described herein combined with ibuprofen are administered or applied concurrently to the patient.

### Long-Acting Local Anesthetics

In certain embodiments, the compositions described herein are administered or applied to a patient in combination with at least one long-acting local anesthetic. EXPAREL® injection (bupivacaine liposome injectable suspension) is a formulation of bupivacaine in a liposomal form that offers patients up to 72 hours of anesthesia and can significantly decrease the need for opioids (See, Bouloux et al. J Oral Maxillofacial Surg. 1999; 57:510).

### Additional Drugs

In certain embodiments, the compositions described herein are administered or applied to a patient in combination with at least one active ingredient drug. For example, the drug(s) is an antimicrobial, such as an antibiotic. In certain embodiments, the antibiotic is selected form a group consisting of: penicillin, amoxicillin, erythromycin, clindamycin, chlorhexidine, and tetracyclines, such as doxycycline. In certain embodiments, the drug(s) is an antifungal. In certain embodiments, the antifungal is nystatin.

### III. Formulations and Doses

Embodiments of the compositions described herein are prepared as formulations, such as sprays, drops, pellets, pills, gels, pastes, balms or rinses. A formulation may be applied topically, which includes as non-limiting examples sublingually, as a rinse, as a gel, as a spray, as a paste, as a pill and/or any manner prescribed by a medical practitioner. The inventive formulation can be administered or applied at a dosage amount, application time, and regimen as prescribed by a medical practitioner. In certain embodiments, the compositions of the present disclosure can be prepared as sterile injectable formulation.

### Spray or Drops

In various embodiments, the compositions described herein are formulated as drops or a spray, which may also be referred to as a “sublingual spray” or “spray.” In some embodiments, the formulation of the spray can be the same to that of the drops except to the extent of the packaging or delivery mechanism. Therefore, any formulation presented in the form of a spray can likewise be formulated as drops, unless otherwise indicated. Any formulation presented in the form of drops can likewise be formulated as a spray, unless otherwise indicated.

In certain embodiments, any composition of the present disclosure which is presented in the form of a spray or drops can likewise be prepared as sterile injectable formulation.

In certain embodiments, drops or spray of the compositions described herein include attenuations of the following substances: Hepar sulphuris calcareum (also known as Calcium sulphide of Hahnemann, and sulphur of lime), Calendula officinalis (also known as Calendule, Calendula, or C. officinalis), Chamomilla (also known as Matricaria recutita or chamomile), Aconitum napellus (also known as monkshood, aconite, wolfsbane, fuzi, monk's blood, or acon), Echinacea angustifolia (also known as or Echinacea or E. angustifolia), Echinacea purpurea (sometimes referred to as Brauneria purpurea (L.) Britton, Helichroa purpurea (L.) Raf. and/or Eastern purple coneflower), Ruta graveolens (also known as or rue or R. graveolens), Arnica montana (also known as or Arnica or A. montana), Mercurius vivus (also known as Quicksilver), Hypericum (also known as St. John's wort), Gelsemium, Ignatia amara (also known as Ignatia, Ignatia amara, samara, Strychnos ignatii, or S. ignatii), or any combination thereof, and optionally additional substances.

In certain embodiments, the drops or spray may include attenuations of Arnica montana, Calendula officinalis, Chamomilla, Echinacea angustifolia, and Ignatia amara. In certain embodiments, the drops or spray include Arnica montana, Calendula officinalis, Chamomilla, Echinacea purpurea, and Ignatia amara. These embodiments may also include additional optional substances.

In certain embodiments, the drops or spray may include attenuations of Arnica montana, Calendula officinalis, Chamomilla, Echinacea angustifolia, Ignatia amara, and Hepar sulphuris calcareum. In certain embodiments, the drops or spray may include attenuations of Arnica montana, Calendula officinalis, Chamomilla, Echinacea purpurea, Ignatia amara, and Hepar sulphuris calcareum. In certain embodiments, the drops or spray may include attenuations of Arnica montana, Calendula officinalis, Chamomilla, Echinacea angustifolia, Ignatia amara, Hepar sulphuris calcareum, and Hypericum. In certain embodiments, the drops or spray may include attenuations of Arnica montana, Calendula officinalis, Chamomilla, Echinacea purpurea, Ignatia amara, Hepar sulphuris calcareum, and Hypericum. In certain embodiments, the drops or spray includes attenuations of Arnica montana, Calendula officinalis, Chamomilla, Echinacea angustifolia, Ignatia amara, Hepar sulphuris calcareum, Hypericum, and Mercurius vivus. In certain embodiments, the drops or spray includes attenuations of Arnica montana, Calendula officinalis, Chamomilla, Echinacea purpurea, Ignatia amara, Hepar sulphuris calcareum, Hypericum, and Mercurius vivus. In certain embodiments, the drops or spray includes attenuations of Arnica montana, Calendula officinalis, Chamomilla, Echinacea angustifolia, Ignatia amara, Hepar sulphuris calcareum, Hypericum, Mercurius vivus, and Ruta graveolens. In certain embodiments, the drops or spray includes attenuations of Arnica montana, Calendula officinalis, Chamomilla, Echinacea purpurea, Ignatia amara, Hepar sulphuris calcareum, Hypericum, Mercurius vivus, and Ruta graveolens. In certain embodiments, the drops or spray includes attenuations of Arnica montana, Calendula officinalis, Chamomilla, Echinacea angustifolia, Ignatia amara, Hepar sulphuris calcareum, Hypericum, Mercurius vivus, Ruta graveolens, and Plantago major. In certain embodiments, the drops or spray includes attenuations of Arnica montana, Calendula officinalis, Chamomilla, Echinacea purpurea, Ignatia amara, Hepar sulphuris calcareum, Hypericum, Mercurius vivus, Ruta graveolens, and Plantago major. In certain embodiments, the drops or spray includes attenuations of Arnica montana, Calendula officinalis, Chamomilla, Echinacea angustifolia, Ignatia amara, Hepar sulphuris calcareum, Hypericum, Mercurius vivus, Ruta graveolens, Plantago major, and Ledum palustre. In certain embodiments, the drops or spray includes attenuations of Arnica montana, Calendula officinalis, Chamomilla, Echinacea purpurea, Ignatia amara, Hepar sulphuris calcareum, Hypericum, Mercurius vivus, Ruta graveolens, Plantago major, and Ledum palustre. In certain embodiments, the drops or spray includes attenuations of Arnica montana, Calendula officinalis, Chamomilla, Echinacea angustifolia, Ignatia amara, Hepar sulphuris calcareum, Hypericum, Mercurius vivus, Ruta graveolens, Plantago major, Ledum palustre, and Silicea. In certain embodiments, the drops or spray includes attenuations of Arnica montana, Calendula officinalis, Chamomilla, Echinacea purpurea, Ignatia amara, Hepar sulphuris calcareum, Hypericum, Mercurius vivus, Ruta graveolens, Plantago major, Ledum palustre, and Silicea.

In certain embodiments, any of the drops or spray formulations of the present disclosure can contain a composition described herein and may optionally contain additional substances. The drops or spray are absorbed through the sublingual mucosa to systemically decrease pain and swelling/edema, including in the oral cavity. In certain embodiments, the use of the drops or spray shortens the time of and duration of maximum pain, inflammation, and/or trismus after a dental surgery. Additionally, when used in combination with the compositions described herein formulated as a rinse and, separately, as a gel, use of the drops or spray after dental surgery reduce the needed dosage of narcotics when used in combination.

A drops or spray formulation may be made by combining substances with optional substances. For example, Hepar sulphuris calcareum, Calendula officinalis, Chamomilla, Aconitum napellus, Echinacea angustifolia, Echinacea purpurea, Ruta graveolens, Arnica montana, Mercurius vivus, Hypericum, Gelsemium, and Ignatia amara are combined and mixed vigorously. Inactive substances, citric acid, glycerin, potassium sorbate and purified water, are then added and blended in the mixture.

In certain embodiments, the drops or spray include attenuations of the following in the following amounts: Hepar sulphuris calcareum having a dilution of 8X; Calendula officinalis having a dilution of 3X; Chamomilla having a dilution of 5X; Aconitum napellus having a dilution of 3X; Echinacea angustifolia having a dilution of 3X; Echinacea purpurea having a dilution of 3X; Ruta graveolens having a dilution of 12X; Arnica montana having a dilution of 3X, 6X, 18X; Mercurius vivus having a dilution of 10X; Hypericum having a dilution of 10X; Gelsemium having a dilution of 30X; and Ignatia amara having a dilution of 30X. A dilution of 3X, 6X, 18X means that a dilution of 3X is present and a dilution of 6X is present and a dilution of 18X is present in the same formulation. In some instances, substances are present in a formulation in one or more dilutions. Patients respond differently to some dilutions. The inclusion of more than one dilution of a particular ingredient will allow for optimum responses across a wider set of patients.

In certain embodiments, the drops or spray include attenuations of the following substances in the following amounts: Arnica montana having a dilution of 3X, 6X, 18X; Calendula officinalis having a dilution of 3X; Chamomilla having a dilution of 5X; Echinacea angustifolia having a dilution of 3X; and Ignatia amara having a dilution of 30X. In certain embodiments, the drops or spray include attenuations of the following substances in the following amounts: Arnica montana having a dilution of 3X, 6X, 18X; Calendula officinalis having a dilution of 3X; Chamomilla having a dilution of 5X; Echinacea purpurea having a dilution of 3X; and Ignatia amara having a dilution of 30X.

In certain embodiments, the drops or spray include attenuations of the following in the following amounts: Hepar sulphuris calcareum having a dilution of 8X; Calendula officinalis having a dilution of 3X; Chamomilla having a dilution of 5X; Ledum palustre having a dilution of 6X; Echinacea angustifolia having a dilution of 3X; Echinacea purpurea having a dilution of 3X; Ruta graveolens having a dilution of 12X; Arnica montana having a dilution of 3X, 6X, 18X; Mercurius vivus having a dilution of 10X; Hypericum having a dilution of 10X; Silicea having a dilution of 8X; and Ignatia amara having a dilution of 30X.

In certain embodiments, the use of the spray or drops shortens the time of, the intensity of, and the duration of maximum pain, inflammation, and/or trismus after a dental surgery. Additionally, when used in combination with the compositions described herein formulated as a rinse and, separately, a gel, embodiments of the spray described herein reduces the necessary dosage of narcotics (such as opioids) when both are used in combination.

The spray may be formulated for application to the inside of the throat of a subject. For example, the throat spray may be used for treatment of tonsillar stones and post-throat surgery, such as tonsillectomies or uvuloplasties, pain and swelling management. In certain embodiments, a combination of the throat spray and a mouth rinse is used to treat tonsillar stones and post-throat surgery, such as tonsillectomies or uvuloplasties, pain and swelling management.

In certain embodiments, the drops or spray of the present disclosure include a tonsillar spray. In certain embodiments, the tonsillar spray has one or more of the following properties: anti-inflammatory, analgesic and/or antimicrobial. In certain embodiments, the tonsillar spray can be used in treatments before, during and/or after tonsillar/uvular surgery. In certain embodiments, the tonsillar spray can be used to exfoliate and/or reduce tonsillar stones. In certain embodiments, the tonsillar spray can be administered in combination with an oral rinse of the present disclosure.

In certain embodiments, the tonsillar spray includes attenuations of the following substances: Arnica montana (also known as or Arnica or A. montana), Calendula officinalis (also known as Calendule, Calendula, or C. officinalis), Chamomilla (also known as Matricaria recutita or chamomile), Echinacea angustifolia (also known as or Echinacea or E. angustifolia), Hepar sulphuris calcareum (also known as Calcium sulphide of Hahnemann, and sulphur of lime), Argentum nitricum (also known as or A. nitricum, Arg-n, Arg-nit or silver nitrate), Calcarea carbonica (also known as C. carbonica, talc, calc-carb or calcium carbonate), Capsicum annuum (also known as C. annuum or caps), Causticum (also known as caust, blended slaked lime or sulphate of potash), Lac caninum (also known as L. caninum, Lac-c, or canine milk), Lachesis muta (also known as L. muta, Lachesis, Lac-c, bushmaster snake venom or surucucu snake venom), Phytolacca decandra (also known as Phytolacca), and Rhus-toxicodendron (also known as Rus-t or Rhus-tox).

In certain embodiments, the tonsillar spray includes attenuations of the following substances: Arnica montana having a dilution of 3X, Argentum nitricum having a dilution of 30X, Calcarea carbonica having a dilution of 6X, Calendula officinalis having a dilution of 3X, Capsicum annum having a dilution of 5X, Causticum having a dilution of 6X, Chamomilla having a dilution of 5X, Echinacea angustifolia having a dilution of 2X, Hepar sulphuris calcareum having a dilution of 6X, Lachesis having a dilution of 12X, Lac caninum having a dilution of 8X, Phytolacca having a dilution of 5X, and Rhus-toxicodendron having a dilution of 12X.

### Gel

In various embodiments, the compositions described herein are formulated as a gel. In certain embodiments, the gel includes attenuations of the following substances: Hepar sulphuris calcareum, Calendula officinalis, Chamomilla, Aconitum napellus, Echinacea angustifolia, Echinacea purpurea, Ruta graveolens, Arnica montana, Mercurius vivus, Hypericum, Gelsemium, Ignatia amara, and optionally additional substances. The gel can be used locally and have local effects. The gel can be applied topically. The gel can be applied directly on the oral mucosa. Moreover, the gel can be absorbed systemically as well and have systemic effects. The gel placed on the oral mucosa provides an alternative to injection into the area.

A gel formulation may be made by preparing a 1 X attenuation called the mother concentrate or mother tincture. A gel is further prepared by mixing purified water with hydroxyethyl cellulose to which the mother concentrate is added along with a sweetener, a skin conditioning, a flavoring agent and preservative. For example, a mother concentrate is made with Hepar sulphuris calcareum, Calendula officinalis, Chamomilla, Aconitum napellus, Echinacea angustifolia, Echinacea purpurea, Ruta graveolens, Arnica montana, Mercurius vivus, Hypericum, Gelsemium, and Ignatia amara. This mother concentrate is then added to a mixture of purified water and hydroxyethyl cellulose. Finally, allantoin, hydroxyethyl cellulose, peppermint oil, purified water, sodium benzoate and xylitol are then combined with the mother concentrate, purified water and hydroxyethyl cellulose.

In certain embodiments, the gel includes attenuations of the following in the following amounts: Hepar sulphuris calcareum having a dilution of 8X; Calendula officinalis having a dilution of 3X; Chamomilla having a dilution of 5X; Aconitum napellus having a dilution of 3X; Echinacea angustifolia having a dilution of 3X; Echinacea purpurea having a dilution of 3X; Ruta graveolens having a dilution of 12X; Arnica montana having a dilution of 3X, 6X, 18X; Mercurius vivus having a dilution of 10X; Hypericum having a dilution of 10X; Gelsemium having a dilution of 30X; and Ignatia amara having a dilution of 30X. These embodiments may also optionally include additional substances.

In certain embodiments, the gel may include attenuations of Arnica montana, Calendula officinalis, Chamomilla, Echinacea angustifolia, and Ignatia amara, and optionally additional substances. In certain embodiments, the gel may include attenuations of Arnica montana, Calendula officinalis, Chamomilla, Echinacea purpurea, and Ignatia amara, and optionally additional substances.

In certain embodiments, the gel may include attenuations of Arnica montana, Calendula officinalis, Chamomilla, Echinacea angustifolia, Ignatia amara, and Hepar sulphuris calcareum. In certain embodiments, the gel may include attenuations of Arnica montana, Calendula officinalis, Chamomilla, Echinacea purpurea, Ignatia amara, and Hepar sulphuris calcareum. In certain embodiments, the gel may include attenuations of Arnica montana, Calendula officinalis, Chamomilla, Echinacea angustifolia, Ignatia amara, Hepar sulphuris calcareum, and Hypericum. In certain embodiments, the gel may include attenuations of Arnica montana, Calendula officinalis, Chamomilla, Echinacea purpurea, Ignatia amara, Hepar sulphuris calcareum, and Hypericum. In certain embodiments, the gel may include attenuations of Arnica montana, Calendula officinalis, Chamomilla, Echinacea angustifolia, Ignatia amara, Hepar sulphuris calcareum, Hypericum, and Mercurius vivus. In certain embodiments, the gel may include attenuations of Arnica montana, Calendula officinalis, Chamomilla, Echinacea purpurea, Ignatia amara, Hepar sulphuris calcareum, Hypericum, and Mercurius vivus. In certain embodiments, the gel includes attenuations of Arnica montana, Calendula officinalis, Chamomilla, Echinacea angustifolia, Ignatia amara, Hepar sulphuris calcareum, Hypericum, Mercurius vivus, and Ruta graveolens. In certain embodiments, the gel includes attenuations of Arnica montana, Calendula officinalis, Chamomilla, Echinacea purpurea, Ignatia amara, Hepar sulphuris calcareum, Hypericum, Mercurius vivus, and Ruta graveolens. In certain embodiments, the gel may include attenuations of Arnica montana, Calendula officinalis, Chamomilla, Echinacea angustifolia, Ignatia amara, Hepar sulphuris calcareum, Hypericum, Mercurius vivus, Ruta graveolens, and Plantago major. In certain embodiments, the gel may include attenuations of Arnica montana, Calendula officinalis, Chamomilla, Echinacea purpurea, Ignatia amara, Hepar sulphuris calcareum, Hypericum, Mercurius vivus, Ruta graveolens, and Plantago major. In certain embodiments, the gel includes attenuations of Arnica montana, Calendula officinalis, Chamomilla, Echinacea angustifolia, Ignatia amara, Hepar sulphuris calcareum, Hypericum, Mercurius vivus, Ruta graveolens, Plantago major, and Ledum palustre. In certain embodiments, the gel may include attenuations of Arnica montana, Calendula officinalis, Chamomilla, Echinacea purpurea, Ignatia amara, Hepar sulphuris calcareum, Hypericum, Mercurius vivus, Ruta graveolens, Plantago major, and Ledum palustre. In certain embodiments, the gel may include attenuations of Arnica montana, Calendula officinalis, Chamomilla, Echinacea angustifolia, Ignatia amara, Hepar sulphuris calcareum, Hypericum, Mercurius vivus, Ruta graveolens, Plantago major, Ledum palustre, and Silicea. In certain embodiments, the gel may include attenuations of Arnica montana, Calendula officinalis, Chamomilla, Echinacea purpurea, Ignatia amara, Hepar sulphuris calcareum, Hypericum, Mercurius vivus, Ruta graveolens, Plantago major, Ledum palustre, and Silicea. These embodiments may also optionally include additional substances.

In certain embodiments, the gel includes attenuations of the following in the following amounts: Arnica montana having a dilution of 3X, 6X, 18X; Calendula officinalis having a dilution of 3X; Chamomilla having a dilution of 5X; Echinacea angustifolia having a dilution of 3X; and Ignatia amara having a dilution of 30X. In certain embodiments the gel includes attenuations of the following in the following amounts: Arnica montana having a dilution of 3X, 6X, 18X; Calendula officinalis having a dilution of 3X; Chamomilla having a dilution of 5X; Echinacea purpurea having a dilution of 3X; and Ignatia amara having a dilution of 30X.

In certain embodiments, the gel includes attenuations of the following in the following amounts: Hepar sulphuris calcareum having a dilution of 8X; Calendula officinalis having a dilution of 3X; Chamomilla having a dilution of 5X; Ledum palustre having a dilution of 6X; Echinacea angustifolia having a dilution of 3X; Echinacea purpurea having a dilution of 3X; Ruta graveolens having a dilution of 12X; Arnica montana having a dilution of 3X, 6X, 18X; Mercurius vivus having a dilution of 10X; Hypericum having a dilution of 10X; Silicea having a dilution of 8X; and Ignatia amara having a dilution of 30X. These embodiments may also optionally include additional substances.

The gel has a formula that allows direct delivery and absorption of active ingredients through oral mucosa. In the studies performed, it has been shown to significantly reduce edema, pain, and swelling associated with surgeries, and to provide immediate relief for Aphthous ulcers and cold sores. The gel has been shown to resolve minor ulcers within 48 hours and major ulcers within 3-4 days while providing immediate pain relief. The normal course for an Aphthous ulcer left untreated is 10 to 14 days.

The gel has shown clinical efficacy for lesions associated with autoimmune inflammatory conditions, such as lichen planus, pemphigoid and pemphigus. The gel also has been shown to accelerate healing of cutaneous lesion associated with surgery or trauma. It has been applied to post-surgical wounds and decreased skin healing in half as compared to placebo. In some embodiments, the gel may be applied to oral cancer patients for treatment of severe xerostomia and/or mucositis after chemotherapy, radiation treatment, and/or surgery.

In certain embodiments, any of the gel formulations of the present disclosure can be formulated as a sub-lingual spray. Any of the gel formulations may optionally include additional substances. A gel may be formulated with additional substances to be applied for veterinary uses. In certain embodiments, the gel is formulated for application to the skin. For example, the gel may be applied to skin for treatment of a burn, an open wound, and a post-surgical incision line.

### Rinse

In various embodiments, the compositions described herein are formulated as a rinse. U.S. Pat. No. 5,376,374 (Zelaya), which is hereby incorporated by reference in its entirety, teaches an oral rinse including “ . . . Tincture of Calendula officinalis (active ratio 1/5, alcohol 65% by vol.) 56 ml, Tincture of Capsicum frutescens (cayenne pepper, active ratio 1/10, alcohol 75% by vol) 10 ml, Tincture of Echinacea purpurea and Echinacea angustifolia (active ratio 1/1.8, alcohol 60% by vol). 56 ml, Tincture of Hydrastis canadensis (goldenseal root, active ratio 1/5, alcohol 40% by vol.) 56 ml, Extract of propolis (alcohol-free, active ratio 1/10) 84 ml, Apple cider vinegar 486 ml, Distilled water 224 ml.” In certain embodiments, propolis is an inactive ingredient in the composition described herein.

Further, GB 1314136, which is hereby incorporated by reference in its entirety, shows Azadirachta indica for applications in a mouthwash. U.S. Pat. No. 9,545,429, which is hereby incorporated herein by reference in its entirety, also teaches a topical formulation including Azadirachta indica.

In certain embodiments, the rinse includes attenuations of the following: Calendula officinalis, Echinacea purpurea, Plantago major, Azadirachta indica (also known as Neem) and optionally additional substances. In certain embodiments, the rinse includes attenuations of Azadirachta indica, Calendula officinalis, and one or both of Echinacea angustifolia and/or Echinacea purpurea. In certain embodiments, the rinse includes attenuations of Azadirachta indica, Calendula officinalis, Plantago major and one or both of Echinacea angustifolia and/or Echinacea purpurea. These embodiments may also optionally include additional substances, such as inactive ingredients.

In certain embodiments, the rinse includes attenuations of the following substances in the following amounts: Calendula officinalis having a dilution of 1X, Echinacea purpurea having a dilution of 1X, Plantago major having a dilution of 1X, Azadirachta indica (also known as Neem) having a dilution of 1X, and optionally additional substances, such as inactive ingredients. In certain embodiments, the rinse includes attenuations of the following substances in the following amounts: Calendula officinalis having a dilution of 1X, Echinacea purpurea having a dilution of 1X, and Azadirachta indica (also known as Neem) having a dilution of 1X, and optionally additional substances, such as inactive ingredients.

A dental rinse formulation may be made by combining a mother tincture with inactive ingredients and additional optional substances. For example, a mother tincture is a combination of Calendula officinalis, Echinacea purpurea, Plantago major, and Azadirachta indica (also known as Neem). Mother tinctures are then measured and combined in a large glass beaker. In another vessel, purified water, glycerin, allantoin and xylitol are mixed together until dissolved, the mother mixture is then added with more mixing, flavoring such as menthol or peppermint oil are dissolved in a solubilizing agent such as Poloxamer 407 or Polysorbate 20 and then added to the mixture and mixed until well combined and clear. Preservatives, for example citric acid and/or potassium sorbate, are then added and the PH adjusted.

In certain embodiments, the rinse has been observed to reduce microbial activity by at least 99%. In certain embodiments, the rinse has been observed to reduce microbial activity by at least 90%. In certain embodiments, the rinse has been observed to reduce microbial activity by at least 80%. In certain embodiments, the rinse has been observed to reduce microbial activity by at least 70%. In certain embodiments, the rinse has been observed to reduce microbial activity by at least 60%. In certain embodiments, the rinse has been observed to reduce microbial activity by at least 50%.

In some embodiments, a combination of a gel and a rinse are applied to a subject for the treatment of an autoimmune inflammatory condition, for example, pemphigus, pemphigoid, or erosive lichen planus. In some embodiments, a combination of a sublingual spray and a rinse are applied to a subject for the treatment of an autoimmune inflammatory condition, for example, pemphigus, pemphigoid, or erosive lichen planus.

In certain embodiments, the rinse of the present disclosure includes an advanced healing & protecting (AHP) oral rinse. In certain embodiments, the AHP oral rinse has one or more of the following properties: radioprotective, soothing, anti-inflammatory and antimicrobial properties. In certain embodiments, the AHP oral rinse can be used in treatments before, during and/or after chemotherapy or radiation therapy for Head & Neck (H&N) cancer patients (such as intraoral squamous cell carcinoma). In certain embodiments, the AHP oral rinse can be used to decrease in certain side-effects from the radiation/chemo treatment, including ulcers, mucositis and dry mouth. In certain embodiments, the AHP oral rinse can be administered in combination with an oral gel or sublingual spray of the present disclosure.

In certain embodiments, the AHP oral rinse includes attenuations of the following substances: Azadirachta indica, Calendula officinalis, Echinacea purpurea, Plantago major, Apis mellifica (also known as A. mellifica or Apis), Arsenicum album (also known as Ars, arsenic, arsenic trioxide, arsenous acid anhydride or arsenous oxide), Aristolochia clematitis (also known as A. clematitis, arist-clem or birthwort), Crown lechleri (also known as dragons blood or blood of the dragon), Okoubaka aubrevillei (also known as Okoubaka or O. aubrevillei), Phosphorous (also known as phos), Xanthoxylum (also known as Zanthoxylum or prickly ash), Strontium carbinicum (also known as stront-c or strontium carbonate), and Thuja occidentalis (also known as T. occidentalis, thuj or tree of life).

In certain embodiments, the AHP oral rinse includes attenuations of the following substances: Azadirachta indica having a dilution of 6X, Calendula officinalis having a dilution of 1X, Echinacea purpurea having a dilution of 1X, Plantago major having a dilution of 1X, Apis mellifica having a dilution of 12X, Arsenicum album having a dilution of 30X, Aristolochia clematitis having a dilution of 5X, Croton lechleri having a dilution of 1X, Okoubaka aubrevillei having a dilution of 6X, Phosphorous having a dilution of 12X, Xanthoxylum having a dilution of 1X, Strontium carbinicum having a dilution of 30X, and Thuja occidentalis having a dilution of 3X.

### Pastes

In certain embodiments, the compositions described herein are formulated as a paste. In certain embodiments, the compositions described herein are formulated as a toothpaste. In certain embodiments, the toothpaste has one or more of the following properties: minimally foaming, all-natural, encourages faster healing, reduces bleeding, freshens breath, good smell, soothing taste, non-gritty, silky, antimicrobial properties, tartar control, tooth whitening, reduction of tooth sensitivity, and re-mineralizing. In certain embodiments, the toothpaste can be used in treating periodontal disease. In certain embodiments, the toothpaste can be used to improvement periodontal pocket depth and reduced gum bleeding.

In certain embodiments, pastes of the present disclosure include attenuations of the following substances: Calendula officinalis, Echinacea angustifolia, Copaiva Officinalis (also known as Copaiba), Baobab.

In certain embodiments, pastes of the present disclosure can include one or more of the following ingredients: Natrum Bicarbonicum (also known as sodium bicarbonate or baking soda, preferably non-aluminum NaHCO₃), bentonite clay, Coral Calcium, Colloidal silver (also known as silver hydrosol), probiotics, castile soap, xylitol (birch), Coenzyme Q10 (also known as CoQ10), Coconut Oil, turmeric, or Silica.

In certain embodiments, pastes of the present disclosure include attenuations of the following substances: Calendula officinalis having a dilution of 1X, Echinacea angustifolia having a dilution of 3X, Copaiba oil having a dilution of 1X, and Baobab having a dilution of 1X.

In certain embodiments, pastes of the present disclosure include the following ingredients: 1X attenuation of Calendula officinalis, 3X attenuation of Echinacea angustifolia, 1X attenuation of Copaiba oil, 1X attenuation of Baobab, baking soda (preferably non-aluminum NaHCO₃), bentonite clay, coral calcium, colloidal silver, probiotics, castile soap, xylitol, Coenzyme Q10, Coconut Oil, turmeric, and Silica.

### Balms and Ointments

In various embodiments, the compositions described herein are formulated as a balm or ointment. In various embodiments, the compositions described herein are formulated as a balm or ointment for topical application. In certain embodiments, the balm or ointment has one or more of the following properties: cutaneous application, decreased inflammation, encourages healing, decreased pain, and decreased scar formation. In certain embodiments, the toothpaste can be used in treating topical laser procedures or other general skin-burn trauma. In certain embodiments, the toothpaste can be used to encourage of skin healing, pain management, reepithalization, preventing scar formation and tissue contraction, and increased Second Intention healing after skin trauma.

In certain embodiments, balms or ointments of the present disclosure include attenuations of the following substances: Apis mellifica, Arsenicum album, Calendula Officinalis, Cantharis, Carbolicum acidum (also known as carb-ac, carbolic acid, phenol, phenyl hydroxide, or phenyl alcohol), Causticum, Graphites (also known as plumbago or black lead), Hypericum, Plantago, and Thiosinaminum (also known as allyl sulphocarbamide or oil of mustard-seed).

In certain embodiments, balms or ointments of the present disclosure include attenuations of the following substances: Apis mellifica having a dilution of 8X, Arsenicum album having a dilution of 8X, Calendula Officinalis having a dilution of 2X, Cantharis having a dilution of 5X, Carbolicum acidum having a dilution of 10X, Causticum having a dilution of 5X, Graphites having a dilution of 6X, Hypericum having a dilution of 5X, Plantago having a dilution of 2X, and Thiosinaminum having a dilution of 3X.

In certain embodiments, balms or ointments of the present disclosure can include Aloe Vera Butter.

### Alternative Dosage Forms

In other embodiments, the composition is formulated as a solution, an oral tablet, a pill, a capsule, a gel cap, a syrup, an elixir, a suspension, an emulsion, a powder, a globule, a lozenge, an aerosol, a paste, a film, a pill or a pellet. In certain embodiments, the compositions of the present disclosure can be prepared as sterile injectable formulation.

In certain embodiments, at least one medication may be administered as a combination therapy with the compositions described herein. For example, the medication is selected from a fluoride, an antiseptic, or a saliva substitute.

### IV. Kits

In some embodiments, the compositions described herein are provided in a kit including one or more of: a spray, drops, pellets, a gel, a paste, a balm, a pill, a pellet or a rinse. In some embodiments, the compositions described herein are provided in a kit including on or more of: a spray/drops/pellets, a gel, a paste, a rinse, or a balm/ointment. In some embodiments, the kit includes a spray and a gel. In some embodiments, the kit includes a spray and a rinse. In some embodiments, the kit includes a gel and a rinse. In some embodiments, the kit includes a paste and a rinse. In some embodiments, the kit includes a paste and a spray. In other embodiments, a kit includes drops or a spray, a gel, and a rinse. In some embodiments, the kit includes drops or spray and a gel. In some embodiments, the kit includes drops or spray and a rinse. In some embodiments, the kit includes one or more containers. In some embodiments, the kit includes one or more containers which individually contain one or more compositions of the present disclosure.

The dosage amounts and dosage routine of each of the spray or drops, gel and rinse can be the same or different. In some embodiments, the kits including a spray, a gel and a rinse can have the following substances in the dilutions listed in Table 4.

TABLE 4
Kit 1

| DROPS/SPRAY | GEL | RINSE |
|---|---|---|
| Hepar sulphuris calcareum 8X, | Hepar sulphuris calcareum 8X, | Calendula officinalis 1X, |
| Calendula officinalis 3X, | Calendula officinalis 3X, | Echinacea purpurea 1X, |
| Chamomilla 5X, | Chamomilla 5X, | Plantago major 1X, |
| Ledum palustre 6X, | Ledum palustre 6X, | Azadirachta indica/Neem 1X, |
| Echinacea angustifolia 3X OR | Echinacea angustifolia 3X OR | |
| Echinacea purpurea 3X, | Echinacea purpurea 3X, | |
| Ruta graveolens 12X, | Ruta graveolens 12X, | |
| Arnica montana 3X, 6X, 18X, | Arnica montana 3X, 6X, 18X, | |
| Mercurius vivus 10X, | Mercurius vivus 10X, | |
| Hypericum 10X, | Hypericum 10X, | |
| Silicea 8X, | Silicea 8X, | |
| Ignatia amara 30X | Ignatia amara 30X | |

In certain embodiments, a kit includes the substances in the formulations shown in Table 5.

TABLE 5
Kit 2

| DROPS/SPRAY | GEL | RINSE |
|---|---|---|
| Hepar sulphuris calcareum 8X, | Hepar sulphuris calcareum 8X, | Calendula officinalis 1X |
| Calendula officinalis 3X, | Calendula officinalis 3X, | Echinacea purpurea 1X |
| Chamomilla 5X, | Chamomilla 5X, | Plantago major 1X |
| Ledum palustre 6X, | Ledum palustre 6X, | Azadirachta indica/Neem 1X |
| Echinacea angustifolia 3X, | Echinacea angustifolia 3X OR | Inactive ingredients: Xylitol, |
| Ruta graveolens 12X, | Ruta graveolens 12X, | Allantoin, Peppermint oil, |
| Arnica montana 3X, 6X, 18X, | Arnica montana 3X, 6X, 18X, | Glycerin, Citric acid, potassium |
| Mercurius vivus 10X, | Mercurius vivus 10X, | sorbate, Purified Water, |
| Hypericum 10X, | Hypericum 10X, | Polysorbate 20, propolis |
| Silicea 8X, | Silicea 8X, | |
| Ignatia amara 30X | Ignatia amara 30X | |

In certain embodiments, a kit includes the substances in the formulations shown in Table 6.

TABLE 6
Kit 3

| DROPS/SPRAY | GEL | RINSE |
|---|---|---|
| Hepar sulphuris calcareum 8X, | Hepar sulphuris calcareum 8X, | Calendula officinalis 1X, |
| Calendula officinalis 3X, | Calendula officinalis 3X, | Echinacea purpurea 1X, |
| Chamomilla 5X, | Chamomilla 5X, | Plantago major 1X |
| Ledum palustre 6X, | Ledum palustre 6X, | Azadirachta indica/Neem 1X |
| Echinacea purpurea 3X | Echinacea purpurea 3X | Inactive ingredients: Xylitol, |
| Ruta graveolens 12X, | Ruta graveolens 12X, | Allantoin, Peppermint oil, |
| Arnica montana 3X, 6X, 18X, | Arnica montana 3X, 6X, 18X, | Glycerin, Citric acid, potassium |
| Mercurius vivus 10X, | Mercurius vivus 10X, | sorbate, Purified Water, |
| Hypericum 10X, | Hypericum 10X, | Polysorbate 20, propolis |
| Silicea 8X, | Silicea 8X, | |
| Ignatia amara 30X | Ignatia amara 30X | |

In certain embodiments, a composition formulated as either drops/spray, gel, or rinse includes the following: Aconitum nap 3X, Hepar sulphuris calcareum 8X, Calendula officinalis 3X, Chamomilla 5X, Echinacea purpurea 3X, Ruta graveolens 12X, Arnica montana 3X, 6X, or 18X, Mercurius vivus 10X, Hypericum 10X, Ignatia amara 30X, and Gelsemium 30X. For example, the compositions in the formulations shown in Table 7 are included in a kit.

TABLE 7
Kit 4

| DROPS/SPRAY | GEL | RINSE |
|---|---|---|
| Hepar sulphuris calcareum 8X | Hepar sulphuris calcareum 8X | Calendula officinalis 1X |
| Calendula officinalis 3X | Calendula officinalis 3X | Echinacea purpurea 1X |
| Chamomilla 5X | Chamomilla 5X | Plantago major 1X |
| Aconitum napellus 3X | Aconitum napellus 3X | Azadirachta indica/Neem 1X |
| Echinacea angustifolia 3X | Echinacea angustifolia 3X | |
| Echinacea purpurea 3X | Echinacea purpurea 3X | |
| Ruta graveolens 12X | Ruta graveolens 12X | |
| Arnica montana 3X, 6X, 18X | Arnica montana 3X, 6X, 18X | |
| Mercurius vivus 10X | Mercurius vivus 10X | |
| Hypericum 10X | Hypericum 10X | |
| Gelsemium 30X | Gelsemium 30X, | |
| Ignatia amara 30X | Ignatia amara 30X | |

The compositions shown in Table 7 are also referred to as VEGA™, and Kit 4 may also be referred to as an “oral pain recovery kit” or a “VEGA™ oral pain recovery kit.” In Kit 4, Gelsemium and Aconitum napellus replace Silicea and Ledum palustre from Kits 1-3.

In certain embodiments, a kit includes the formulations shown in Table 8.

TABLE 8
Kit 5

| TONSILLAR SPRAY | RINSE |
|---|---|
| Arnica montana 3X | Calendula officinalis 1X, |
| Argentum nitricum 30X | Echinacea purpurea 1X, |
| Calcarea carbônica 6X | Plantago major 1X, |
| Calendula officinalis 3X | Azadirachta indica/Neem 1X, |
| Capsicum annum 5X | |
| Causticum 6X | |
| Chamomilla 5X | |
| Echinacea angustifolia 2X | |
| Hepar sulphuris calcareum 6X | |
| Lachesis 12X | |
| Lac caninum 8X | |
| Phytolacca 5X | |
| Rhus-toxicodendron 12X | |

In certain embodiments, a kit includes a toothpaste formulation shown in Table 9.

TABLE 9
Kit 6 (Toothpaste)

Baobab 1X
Calendula officinalis 1X
Copaiba oil 1X
Echinacea angustifolia 3X
Baking Soda (non-aluminum NaHCO3)
Bentonite Clay
Castile soap
Coconut Oil
Colloidal silver (silver hydrosol)
CoQ10
Coral Calcium
Probiotics
Silica
Tumeric
Xylitol (birch)

In certain embodiments, a kit includes the formulations shown in Table 10.

TABLE 10
Kit 7

| ORAL GEL | AHP ORAL RINSE |
|---|---|
| Hepar sulphuris calcareum 8X, | Azadirachta indica 6X |
| Calendula officinalis 3X, | Calendula officinalis 1X, |
| Chamomilla 5X, | Echinacea purpurea 1X, |
| Aconitum napellus 3X, | Plantago major 1X |
| Echinacea angustifolia 3X, | Apis mellifica 12X |
| Echinacea purpurea 3X | Arsenicum album 30X |
| Ruta graveolens 12X, | Aristolochia clematitis 5X |
| Arnica montana 3X, 6X, 18X, | Croton lechleri 1X |
| Mercurius vivus 10X, | Okoubaka aubrevillei 6X |
| Hypericum 10X, | Phosphorous 12X |
| Gelsemium 30X, | Xanthoxylum 1X |
| Ignatia amara 30X | Strontium carbinicum 30X |
| | Thuja occidentalis 3X |

In certain embodiments, a kit includes the substances in the formulations shown in Table 11.

TABLE 11
Kit 8 (Topical Balm)

Aloe Vera Butter
Apismellifica 8X
Arsenicum album 8X
Calendula Officinalis 2X
Cantharis 5X
Carbolicum acidum 10 X
Causticum 5X
Graphites 6X
Hypericum 5X
Plantago 2X
Thiosinaminum 3X

Potential inactive ingredients include, but are not limited to, citric acid, glycerin, potassium sorbate, and purified water. Other potential inactive ingredients include, but are not limited to, allantoin, hydroxyethyl cellulose, peppermint oil, purified water, sodium benzoate, and xylitol. Still other potential inactive ingredients include, but are not limited to, Xylitol, Allantoin, Peppermint oil, Glycerin, Citric acid, Potassium sorbate, Purified Water, Polysorbate 20, and Poloxamer 407.

Other inactive ingredients may be any ingredient that forms a desirable consistency for the ingredients. Inactive ingredients, such as excipients that are common to the health and/or cosmetic industries, as well as minerals, such as sea minerals can be used. Sea minerals, which can be included in the formulation of the embodiments described herein include, but are not limited to, one or more of the following elements: Antimony, Barium, Beryllium, Bismuth, Boron, Bromine, Cadmium, Calcium, Carbonate, Cerium, Cesium, Chloride, Chromium, Cobalt, Copper, Dysprosium, Erbium, Europium, Gadolinium, Gallium, Germanium, Gold, Hafnium, Holmium, Indium, Iodine, Iridium, Iron, Lanthanum, Lead, Lithium, Lutetium, Magnesium, Manganese, Molybdenum, Neodymium, Nickel, Niobium, Osmium, Palladium, Propolis, Phosphorus, Platinum, Potassium, Praseodymium, Rhenium, Rhodium, Rubidium, Ruthenium, Samarium, Scandium, Selenium, Silicon, Silver, Sodium, Strontium, Sulfate/Sulfur, Tantalum, Tellurium, Terbium, Thallium, Thorium, Thulium, Tin, Titanium, Tungsten, Vanadium, Ytterbium, Yttrium, Zinc, Zirconium, plus other naturally occurring trace minerals.

Preservatives, such as potassium sorbate or silver, can also be included in the formulations described herein.

The compositions herein taught might also be combined with surfactants and or substances such as dimethyl sulfoxide (DMSO) that may promote penetration of the compositions, such as through or beneath the skin. Optional substances are those that when combined with the compositions described herein, result in the same cutaneous or subcutaneous reduction of swelling, bruising, pain. Other examples of some excipients used in accordance with the embodiments described herein include, but are not limited to, viscosity modifying agents, buffers, antioxidants, emulsifying agents, absorbents, antiacne agents, antiperspirants, anticaking agents, antifoaming agents, antimicrobial agents, anti-infective agents, antioxidants, antidandruff agents, astringents, binders, buffers, biological additives, buffering agents, bulking agents, chelating agents, chemical additives, coupling agents, conditioners, colorants, cosmetic astringents, cosmetic biocides, denaturants, drug astringents, detergents, dispersants, external analgesics, film formers, foaming agents, fragrance components, flavoring agents humectants, keratolytics, opacifying agents, pH adjusters, preservatives, propellants, proteins, retinoids, reducing agents, sequestrants, skin bleaching agents, skin-conditioning agents (humectants, miscellaneous, and occlusive), skin soothing agents, skin healing agents, softeners, solubilizing agents, lubricants, penetrants, plasticizers, solvents, co-solvents, sun screening additives, sweeteners, salts, essential oils, and vitamins. In some embodiments, formulations may include additional substances ranging from about 0.01 percent weight (% w/w) to about 95% w/w relative to the total weight of the formulation.

In certain embodiments, the compositions described herein demonstrate antimicrobial properties.

The formulations herein might also be administered or applied simultaneously or during, or as a substitute for, treatment with prescription medications such as NORCO® pills (also known as VICODIN®, and wherein any reference to NORCO® pills throughout shall mean Hydrocodone 5 mg/Acetaminophen 325 mg), DECADRON® 8 mg dexamethasone IV at the time of the surgery, MOTRIN® 600 mg ibuprofen, and/or additional analgesics, steroids, or the like considered appropriate by a skilled practitioner.

### V. Administration and Application

The formulations of the present disclosure, including sprays, pellets, pills, gels, pastes, balms and rinses can be packaged separately or together. In certain embodiments, one or more of the sprays, pellets, pills, gels, pastes, balms or rinses of the present disclosure are individually packaged. In certain embodiments, one or more of the sprays, pellets, pills, gels, pastes, balms or rinses of the present disclosure are individually contained, and then packaged together as a kit. Each of the sprays, pellets, pills, gels, pastes, balms or rinses of the present disclosure can be used together or separately. For example, a method of treating a subject for at least one of the conditions above includes the steps of first administering or applying the rinse, second administering or applying the spray, and third administering or applying the gel.

The formulations can be administered or applied prior to any surgical procedures, for example, any dental procedures. Administering or applying may begin three days before oral surgery and continue to seven days after surgery. In some embodiments, administering or applying begins less than three days before oral surgery. In some embodiments, administering or applying begins two days before oral surgery. In some embodiments, administering or applying begins one day before surgery. In some embodiments, administering or applying begins more than three days before surgery. For example, administering or applying begins four days, five days, six days, seven days, eight days, nine days, ten days, eleven days, twelve days, or thirteen days before surgery. In some embodiments, administering or applying continues less than seven days after surgery. For example, administering or applying continues one day, two days, three days, four days, five days, and six days after surgery. Alternatively, administering or applying continues for more than seven days after surgery. For example, administering or applying continues eight days, nine days, ten days, eleven days, twelve days, thirteen days, fourteen days, fifteen days, sixteen days, or seventeen days after surgery.

Administering or applying may occur, for example, prior to, the day of, and the day or days following the surgery.

The formulations can, for example, be applied every day consecutively or can be applied as needed. The formulations can be used once a day. The formulations can be used two times a day. The formulations can be used three times a day. The formulations can be used four times a day. The formulations can be used five times a day.

In certain embodiments described herein, the composition(s) is administered or applied to the oral mucosa of a subject. The oral mucosa is the mucous membrane lining the inside of the mouth and consists of stratified squamous epithelium termed oral epithelium and an underlying connective tissue termed lamina propria. IgA found therein has a role in the protection against microbes and is also an immunological regulator.

Oral mucosa heals differently than non-mucosal epidermis (Chen et al. BMC Genomics 2010, 11:471, which is hereby incorporated by reference in its entirety). Oral mucosal contains fewer cytokines than skin, which results in significantly reduced inflammatory potential. IL-1b and TNF-α, for example, are reduced in oral mucosal tissue. As used herein “mucosal layer” means any mucous membranes (or mucosae, mucosas, or mucosa) or mucosal tissue.

### Prevention and Treatment

Embodiments described herein are directed toward oral and/or dental care, treatment of throat lesions and related procedures, and cutaneous/skin application. Oral and/or dental care includes, for example, general oral maintenance, reduction of flora in the mouth, reduction of pain, reduction of inflammation, reduction of bruising, trismus, recovery after dental surgeries, amelioration of anxiety associated with dental care or preventative measures for maladies of the oral cavity. Maladies of the oral cavity include, for example, cavities, tooth decay, bone loss, and periodontal pocket formation.

Additional conditions that may be treated by a composition described herein formulated as a gel, drops or a spray, or a rinse, either in a combination therapy or separately, include: dry mouth, oral ulcers, dry sockets, post-bleaching gingival sensitivity, and cold/denture sores. In certain embodiments, the condition is associated with bridge work.

### Inflammation, Swelling, and Pain Reduction

Various embodiments described herein include a method of decreasing or reducing post-operative inflammation, edema/swelling, bruising, pain and related symptoms such as trismus after bodily insult, including after oral surgery, by administering or applying the compositions described herein. For example, the method reduces pain from a bruised/uncomfortable area after a rubber clamp was used during surgery. The active ingredients in the gel, the rinse, and drops or spray, in addition to reducing pain/swelling/bruising, also have anti-anxiety properties.

In Lee et al., a gel, a rinse, and a spray described herein were administered to patients three days before a block bone graft surgery of the mandible (See, Lee et al., Clinical Journal of Pharmacology and Pharmacotherapeutics, Volume 1, Article 1006 (2019), which is hereby incorporated by reference in its entirety). The patients were observed to have less pain for at least 72 hours compared to patients administered only opioid rescue analgesics. In fact, patients administered the rinse, the gel, and the spray had a median time interval to first post-operative use of an opioid rescue analgesic that was twice as long as the median time interval for the patients, who were not administered the gel, the rinse, and the spray (4.3 hours vs. 1.9 hours).

Ignatia amara, which may be part of the gel and drops or spray formulations of the compositions described herein, is known in the art to be a “natural antidepressant”. The rinse has antimicrobial properties and has additive “antibiotic” properties.

In certain embodiments, the treatments herein increase healing and decrease inflammation by 24 hours after surgery or after the initiation of administration or application. In certain embodiments, healing and inflammation are further improved by 48 hours after surgery or after the initiation of administration or application. The compositions described herein may also reduce the length of time a patient experiences swelling following an operation.

In certain embodiments, the compositions described herein are used for treating or preventing mucositis and/or thrush. Each of the compositions may be formulated as a rinse, drops, a spray, pellets, pills or a gel. In some embodiments, a combination therapy includes administering or applying to a subject with mucositis and/or thrush a rinse, drops or a spray, and a gel of at least one composition described herein. In an aspect of an embodiment, the patient may have a long-standing bilateral buccal mucositis. The compositions described in Example 1 have been shown to significantly accelerate healing of a lesion resulting from mucositis and/or thrush. For example, a subject is treated for about one week, about two weeks, about three weeks, about four weeks, or about five weeks. In some embodiments, the subject is treated for about a month with the compositions described herein. Treatment resolves the lesion by about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%.

### Aphthous Ulcers

In certain embodiments, the compositions described herein are administered or applied to a subject for treating aphthous ulcers. Aphthous ulcers, also referred to as “canker sores,” are disproportionately painful relative to the size of the lesion (See, Froum, S.; Perio-Implant Advisory; https://www.perioimplantadvisory.com/articles/2018/08/canker-sores-an-old-enemy-facing-new-treatment.html, which is hereby incorporated by reference in its entirety). Canker sores can arise from factors such as stress, medications, hormonal changes, vitamin deficiency, sensitivities to foods, and dental substances, such as sodium lauryl sulfate found in toothpaste. Specifically, deficiencies of vitamins B12, folic acid, and iron are associated with canker sores. In certain embodiments, the composition described herein for treating aphthous ulcers is formulated as a gel.

### Reducing Microbial Activity

In certain embodiments, the compositions described herein are used to reduce the number of microorganisms or reduce microbial activity. The microorganisms are selected from Streptococcus mutans (S. mutans), Actinomyces viscosus (A. viscosus), Streptococcus pyogenes (S. pyogenes), Porphyromonas gingivalis (P. gingivalis), and Bacteroides fragilis (B. fragilis). Microbial activity may be reduced by at least 30%. For example, microbial activity is reduced by a range of about 30% to about 40%, about 40% to about 50%, about 50% to about 60%, about 60% to about 70%, about 70% to about 80%, about 80% to about 90%, and about 90% to about 100%. In some embodiments, the reduction is about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

All references noted in this application, both patent literature and/or non-patent literature, are incorporated herein by reference in their entirety. References that are not specifically singled out as being incorporated herein by reference in their entirety are nonetheless incorporated herein by reference in their entirety.

### Reducing Opioid Use

In certain embodiments, administering or applying at least one composition described herein reduces post-operative opioid use in a patient as compared to a patient who had undergone the same operation without administration or application the at least one composition. In certain embodiments, the method for reducing opioid prescription and use is the one described in Tatch, Walter; “Opioid Prescribing Can Be Reduced in Oral and Maxillofacial Surgery Practice,” Journal of Oral and Maxillofacial Surgery; available online 19 Mar. 2019; https://doi.org/10.1016/j.joms.2019.03.009; which is hereby incorporated by reference in its entirety.

The operation may occur in the mouth, i.e., oral surgery. In certain embodiments, the composition is formulated as a gel, a rinse, or as drops or a spray. The patient may be administered more than one or more than one may be applied of the rinse, the drops or spray, and the gel. For example, all three of the gel, the rinse, and the drops or spray are administered or applied to the patient. Alternatively, two of the gel, the rinse, and the drops or spray are administered or applied to the patient. In certain embodiments, administering or applying occurs more than once. The step of administering or applying may occur pre-operation and continue post-operation.

Administering or applying the at least one composition described herein has been observed to reduce the use of opioids by at least 10% in a patient recovering from surgery. For example, the reduction is within a range of about 10% to about 20%, about 20% to about 30%, about 30% to about 40%, about 40% to about 50%, about 50% to about 60%, about 60% to about 70%, about 70% to about 80%, about 80% to about 90%, or about 90% to about 100%.

In certain embodiments, opioid use is reduced about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

### VI. Definitions

Allopathic medicine—As used herein, the term “allopathic medicine” refers to physical interventions for treating, suppressing, or preventing symptoms, pathology, or physiological processes associated with a disease, disorder, or condition.

Attenuation—As used herein, the term “attenuation” refers to a liquid preparation of aqueous or hydro-alcoholic tinctures. The terms “dilution” and “potency,” as used herein, refer to the concentration of a certain material within an attenuation.

External use—As used herein, the terms “external use” or “topical use” refer to utilization of a substance on the body's surface resulting in a localized therapeutic effect.

Full recovery—As used herein, the term “full recovery” refers to the absence, or below detectable levels, of symptoms, pathology or physiological processes associated with a disease, disorder, or condition following the introduction of a treatment regimen or intervention, such as administration or application of the compositions of the present disclosure.

Medicinal raw material—As used herein, the term “medicinal raw material” refers to a portion of raw material, which is extracted in a suitable diluent for preparation of a tincture.

Operation—As used herein, the term “operation” refers to a physical procedure performed on a patient to repair damage or remedy an existing defect.

Opioid—As used herein, the term “opioid” refers to a drug substance that acts as an agonist on opioid receptors, often resulting in addictive properties or physiological effects similar to those of opium.

Substance—As used herein, the term “substance” refers to a type of matter that may be used as a component or ingredient in the compositions described herein.

Succuss—As used herein, the terms “succussion” or “succuss” refer to a vigorous agitation of a liquid or mixture.

Tincture—As used herein, the term “tincture” refers to a liquid preparation of a medicinal raw material extracted from a part, combinations of parts, and/or the whole of a raw source material, as combined with a diluent. The term “mother tincture” or “mother concentrate”, as used herein, refers to a tincture of medicinal raw material extracted in a 1X attenuation from the a raw source material, which is the reference material from which further attenuations are made.

Trituration—As used herein, the term “trituration” is a non-water-soluble ingredient in solution.

### VII. Equivalents and Scope

The present disclosure provides different forms and should not be construed as limited to the embodiments set forth herein. Rather, these embodiments are provided so that this disclosure will be thorough and complete, and will fully convey the scope of the disclosure to those skilled in the art.

Those skilled in the art will recognize or be able to ascertain using no more than routine experimentation, many equivalents to the specific embodiments in accordance with the disclosure. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

The term “about” or “approximately” means within an acceptable error range for the value as determined by one of ordinary skill in the art, which will depend in part on how the value is measured or determined, i.e., the limitations of the measurement system. For example, “about” with respect to the compositions can mean plus or minus a range of up to 20%, up to 10%, up to 5%. Alternatively, particularly with respect to biological systems or processes, the term can mean within an order of magnitude, within 5-fold, or within 2-fold, of a value. Where values are described in the application and claims, unless otherwise stated the term “about” means within an acceptable error range for the value. For example, when referring to a period of time, e.g., hours, the present values (±20%) are more applicable. Thus, 6 hours can be, e.g., 4.8 hours, 5.5 hours, 6.5 hours, 7.2 hours, as well as the usual 6 hours. Another example includes a period of time measured in days such that “about” a week can refer to 6 days or 7 days or 8 days or any time in between such as 6 days 12 hours.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

### EXAMPLES

Examples are provided as non-limiting illustrations of the present disclosure, and should in no way be construed limiting the scope of the disclosure in any manner, but rather should be construed to encompass any and all variations which are or will become evident as a result of the disclosure provided herein.

### Example 1. Analysis of Anti-Inflammatory and Analgesic Effects

The anti-inflammatory and analgesic effects of compositions described herein formulated as a spray/drops, a gel, and a rinse (also referred to as a VEGA™ Recovery Kit from StellaLife, Inc.) were analyzed after the surgical removal of four complete bony third molars. The formulations of the compositions administered or applied to the patients are shown in Table 7.

A group of 10 patients (three males and seven females) requiring removal of asymptomatic impacted third molars with Class II/III position C impaction were selected for surgery. All of the patients satisfied the following selection criteria: American Society of Anesthesiologists category 1 (ASA 1) and four full bony impacted (Class II/III position C) third molars. The age of the participants ranged between 17-21 years.

All surgical procedures were performed by the same surgeon, and no NSAIDS or steroids were administered to the patients for at least one month prior to the study.

Administration or application of the composition formulations as described in Table 7 was initiated three days before the surgery and continued for 7 days after the surgery. Patients gargled with the rinse (10-15 cc) three times daily for two to three minutes, and then spit out the rinse.

Patients were sublingually administered 15 drops (spray formulation) containing about 0.07 cc each four times daily. Patients were instructed not to swallow for about one minute. The gel (about 0.02 cc) was applied to the gum surrounding the tooth extraction site three times daily using a cotton swab applicator. Patients did not rinse or eat for 20 minutes after application, allowing for absorption of the gel.

Patients were administered four Amoxicillin (500 mg/tablet) or Clindamycin (150 mg/tablet) tablets one hour before surgery. Patients had the option to take the Amoxicillin or Clindamycin tablets with water.

Facial swelling and trismus were evaluated by measuring facial reference distances and maximum mouth opening size, respectively. Facial swelling and trismus were measured on post-operative days 2, 4, and 7.

Mouth opening size, which is defined as the maximum distance between upper and lower central incisors, was measured by a ruler to the nearest mm, and the pre-operation (pre-op) value was recorded as the baseline. Trismus, which is a reduced opening of the jaws, was measured the same way on days 2, 4 and 7 post-operatively. Table 12 shows the average measurements obtained during assessment of trismus in the patient.

TABLE 12
Trismus/Mouth Opening

| | Days Post-Operation | | | |
|---|---|---|---|---|
| | Pre-Operation | | | |
| Measurements | (baseline) | DAY 2 | DAY 4 | Day 7 |
| mm | 49 | 22.56 | 29.89 | 39.78 |
| % trismus | — | 46% | 61% | 81% |

Facial swelling was measured by a tape measuring method. Four measurements were made between 6 reference points: tragus, lateral canthus of eye, pogonion, ala, gonion and the corner of the mouth. The pre-operation (pre-op) sum of the measurements was recorded. Table 13 shows the measurements of swelling in the patients.

TABLE 13
Swelling

| | Days Post-Operation | | | |
|---|---|---|---|---|
| | Pre-Operation | | | |
| Measurements | (baseline) | DAY 2 | DAY 4 | Day 7 |
| Sum of measurements | 913.75 | 944.88 | 922.75 | 915.63 |
| [mm] | | | | |
| % of baseline | | 103.4% | 101% | 100.2% |
| % change from previous | | 3.4% | 1% | 0.2% |
| measurement | | | | |

Post-operative pain was assessed using a visual analogue scale (VAS) anchored by verbal descriptors. Specifically, post-operative pain was rated daily for 7 days using a 100-point VAS anchored by the verbal descriptors “no pain” (0) and “very severe pain” (100). Pain levels between about 10 to about 30 are indicative of a range of mild pain, pain levels between about 40 to about 60 are indicative of a range of moderate pain, and pain levels between about 70 to 100 are indicative of a range of severe pain. VAS daily pain measurements of post-operative pain are shown in Table 14.

TABLE 14
Daily pain measurements

| | Days Post-Operation | | | | | | |
|---|---|---|---|---|---|---|---|
| | DAY 1 | DAY 2 | DAY 3 | DAY 4 | DAY 5 | DAY 6 | DAY 7 |
| Severity (Patient) | 73.375 | 51.23 | 49.25 | 38.38 | 29.63 | 20 | 10 |
| Severity (Clinician) | — | 57.63 | — | 37 | — | — | 14 |
| Intensity (Patient) | 58.88 | 40.13 | 36.13 | 29.13 | 21.63 | 13.25 | 8.5 |
| Intensity (Clinician) | — | 46.63 | — | 26.25 | — | — | 9.75 |

Post-operative pain was also assessed using patient self-reports on the number of analgesic tablets required for post-operative pain relief. Patients received 15 pills in tablet form of NORCO® (hydrocodone 5 mg/acetaminophen 325 mg) to be used if needed. Table 15 shows the average number of NORCO® (hydrocodone 5 mg/acetaminophen 325 mg) pills taken per patient for the first seven days post-operation.

TABLE 15
NORCO ® pill pain management

| | Days Post-Operation | | | | | | |
|---|---|---|---|---|---|---|---|
| | DAY 1 | DAY 2 | DAY 3 | DAY 4 | DAY 5 | DAY 6 | DAY 7 |
| Average number of NORCO ® pills | 3.5 | 2.3 | 0.8 | 0.6 | 0.5 | 0.4 | 0.25 |

After surgery, each patient was given the HRQOL (also shown herein as Health-Related Quality of Life or HRQL) instrument in a diary as described by Shugars et al. J Oral Maxillofacial Surg 1996, 54: 1402-1408, which is hereby incorporated by reference in its entirety). HRQOL data were collected from the patients on daily bases for 7 days after the procedure (See, White et. al. J OMS, 2003; Issue 5, pp 535-544, Kim et al. 000, 2006; 102:e4; Boulouxet et al. J Oral Maxillofacial Surg. 1999; 57:510; Esen et al. J Oral Maxillofacial Surg. 1999; 57: 1201; Nayyar et al. Br J Oral Maxillofacial Surg. 2006; 44:501; Shugars et al. J Oral Maxillofacial Surg 1996, 54: 1402-1408; each of which is hereby incorporated by reference in its entirety).

The data presented in Tables 8-10 indicate improved post-operative facial swelling, trismus, and pain compared to the baseline values by the fourth day post-operation. Peak swelling occurred on the second post-operative day, followed by a decrease that approached baseline by the fourth post-operative day. Most patients required NORCO® (hydrocodone and acetaminophen) pills for post-operative pain control for the first 2-3 days after the surgery. In fact, by post-operative day 3, the patients required fewer than one NORCO® (hydrocodone and acetaminophen) pill. By the fourth post-operative day, swelling was reduced three-fold compared to the second post-operative day, at which point it was only 1% higher than the pre-operative baseline. Recovery for most HRQOL measures, including pain, occurred by post-operative day four.

In contrast, the study in White et al. was conducted in 630 patients undergoing third molar removal and reported use of over-the-counter and prescribed medications for post-operative care, including pain management (See, J OMS, 2003; Issue 5, pp 535-544, which is hereby incorporated by reference in its entirety). In the study, recovery of most HRQOL measures was observed within the first five days following surgery. However, recovery from pain, in particular, was delayed. By post-operative day seven, 15% of patients continued to report post-operative pain as severe, and 5% reported post-operative pain as average (See, White et al. J OMS, 2003; Issue 5, pp 535-544, which is hereby incorporated by reference in its entirety).

Therefore, the VEGA™ Oral Pain Recovery Kit provided an alternative or supplement to opioids for post-operative care, including improvement of pain management, swelling, and trismus during recovery from third molar extraction surgery.

### Example 2. Assessment of Antimicrobial Activity of Oral Rinse Product

A study was completed using the oral care rinse of Table 7, which includes Calendula officinalis 1X, Echinacea purpurea 1X, Plantago major 1X, and Azadirachta indica/Neem 1X. The rinse was tested for antimicrobial activity against Streptococcus mutans (S. mutans) (ATCC 251175), Actinomyces viscosus (A. viscosus) (ATCC 15987), Streptococcus pyogenes (S. pyogenes) (ATCC 19615), Porphyromonas gingivalis (P. gingivalis) (ATCC 33277), and Bacteroides fragilis (B. fragilis) (ATCC 43858) according to American Society for Testing and Materials (ASTM) International Method E2315, Assessment of Antimicrobial Activity using a Time-Kill Procedure.

Test microorganisms were prepared on appropriate agar plates or liquid culture medium. The suspension of test microorganisms was standardized, as needed, by dilution in a buffered saline solution (PBS). Test and control substances were dispensed in identical volumes (10 ml) to sterile vessels. Independently, Test and Control substances were inoculated with each test microorganism (1 ml), then mixed and incubated. Control substances were immediately harvested and represent the concentration present at the start of the test, or time zero. At the conclusion of the contact time, a volume of the liquid test solution was harvested and chemically neutralized by adding 1 ml of the inoculated test substance to PBS, and then serially diluted. All dilutions were membrane-filtered within five minutes.

Dilutions of the neutralized test solution were assayed using appropriate growth media to determine the surviving microorganisms at the respective contact times. Reductions of microorganisms were calculated by comparing initial microbial concentrations to final microbial concentrations by percent reduction=((B−A)/B×100), where B is the number of viable test microorganisms in the control substance immediately after inoculation, and A is the number of viable test microorganisms in the test substance after the contact time. Alternatively, reduction in microorganisms may be calculated by log 10 reduction=Log(B/A), where B is the number of viable test microorganisms in the control substance immediately after inoculation, and A is the number of viable test microorganisms in the test substance after the contact time.

The average number of viable bacteria recovered from the time zero samples was approximately 1×10⁶ cells/m1 or greater. Ordinary consistency between replicates must be observed for the time zero samples. Positive/growth controls demonstrated growth of the appropriate test microorganisms. Negative/purity controls demonstrated no growth of the test microorganism.

The control substance for S. pyogenes (ATCC 19615), P. gingivalis (ATCC 33277), and B. fragilis (ATCC 43858), and S. mutans (ATCC 251175) was PBS (10 ml), and the control substance for A. viscosus (ATCC 15987) was Dey/Engley Broth (10 ml). The culture growth media for S. pyogenes (ATCC 19615), P. gingivalis (ATCC 33277), S. mutans (ATCC 251175), and B. fragilis (ATCC 43858) was tryptic soy agar (TSA) with 5% sheep's blood, and the culture growth media for A. viscosus (ATCC 15987) was brain heart infusion broth (BHIB).

The inoculum concentration for S. pyogenes (ATCC 19615) and the number of viable bacteria recovered from the time zero samples was 2.50×10⁶ CFU/ml. The inoculum concentration for B. fragilis (ATCC 43858) and the number of viable bacteria recovered from the time zero samples was 1.75×10⁷ CFU/ml and 2.05E+06 CFU/ml. The inoculum concentration for A. viscosus (ATCC 15987) and the number of viable bacteria recovered from the time zero samples was 6.64×10° CFU/ml. The inoculum concentration of S. mutans (ATCC 25175) and the number of viable bacteria recovered from the time zero samples was 3.95E+06 CFU/ml. The inoculum concentration for P. gingivalis (ATCC 33277) and the number of viable bacteria recovered from the time zero samples was 3.30E+06 CFU/ml.

The plating media for S. pyogenes (ATCC 19615), P. gingivalis (ATCC 33277), and B. fragilis (ATCC 43858) was tryptic soy agar (TSA) with 5% sheep's blood, and plating media for A. viscosus (ATCC 15987) was brain heart infusion agar (BHIA). S. pyogenes (ATCC 19615) was incubated at 36±1° C.±5% CO₂ for 24-48 hours. B. fragilis (ATCC 43858) was incubated at 36±1° C. under anaerobic conditions for 24-48 hours. A. viscosus (ATCC 15987) was incubated 36±1° C. for 72-96 hours. P. gingivalis (ATCC 33277) was incubated 36±1° C. under anaerobic conditions for 10 days. Contact time was reduced to 30 minutes for A. viscosus, S. pyogenes, P. gingivalis, and B. fragilis. The test substance was neutralized by adding 1 ml of the inoculated substance to 9 ml of PBS and serially diluted.

Results of the time-kill procedure for S. mutans are shown in Table 16.

TABLE 16
Assessment of Antimicrobial Activity

| | | | Percent | Log10 |
|---|---|---|---|---|
| | | | Reduction | Reduction |
| | | | Compared to | Compared to |
| | Contact | | Control at | Control at |
| S | Time | CFU/ml | Time Zero | Time Zero |
| S. mutans | 45 minutes | 1.60E+04 | 99.59% | 2.39 |
| (ATCC 25175) | 45 minutes | 1.10E+04 | 99.72% | 2.56 |
| | 45 minutes | 1.45E+04 | 99.63% | 2.44 |
| S. pyogenes | 30 minutes | 2.82E+03 | 99.89% | 2.95 |
| (ATCC 19615) | 30 minutes | 1.16E+07 | 33.52% | 0.18 |
| B. fragilis | 30 minutes | 5.08E+05 | 75.22% | 0.61 |
| (ATCC 43858) | | | | |
| A. viscosus | 30 minutes | 6.10E+02 | 99.08% | 2.04 |
| (ATCC 15987) | | | | |
| P. gingivalis | 30 minutes | <5.00E+00 | >99.99% | >5.82 |
| (ATCC 33277) | | | | |

The rinse was observed to reduce microbial activity of S. mutans by an average of 99.65% with an average log 10 reduction of 2.46. A high rinse efficacy was also observed against A. viscosus, S. pyogenes, P. gingivalis, and B. fragilis. The number of viable P. gingivalis bacteria was shown as <5.00E+00 CFU/ml because the limit of detection for this assay was 5.00E+00 CFU/ml.

### Example 3. Post-Surgical Management after Periodontal Surgical Procedures

Twenty patients were evaluated for the effects of the gel and rinse described in Table 7 of Example 1, as an adjunct therapy to regular brushing and flossing, for post-surgical management following pocket reduction osseous surgery, connective tissue grafting, extraction, and/or a site preservation bone graft. Patients were ASA class I or class II with moderate periodontitis defined as periodontal pockets with a depth of 4-6 mm.

The twenty patients were not on any medication prior to undergoing pocket reduction osseous surgery, connective tissue grafting, extraction, and/or site preservation bone graft. The gel and rinse were applied as an adjunct therapy on one side in the split-mouth designs after surgery. On the opposite side of the mouth, either PERIDEX® rinse (chlorhexidine gluconate 0.12%) or an AO PERIOSCIENCE® rinse) was applied.

After three days, less swelling was observed on the compositions-treated side than on the side treated with either the PERIDEX® rinse (chlorhexidine gluconate 0.12%) or the AO PERIOSCIENCE® rinse.

Patients unanimously reported reduced swelling and pain on the gel and rinse-treated side compared to the side treated with either the PERIDEX® rinse (chlorhexidine gluconate 0.12%) or the AO PERIOSCIENCE® rinse.

### Example 4. Reduction of Periodontal Pocket Depth

This example was conducted to determine the effects of treatment with the compositions described herein on periodontal pocket depth in patients with moderate periodontal disease who had undergone deep scaling and root planing. Pocket depths were measured prior to treatment. A split-mouth study design was used to determine the reduction of periodontal pocket depth after one month of gel (0.02 ounces for each application) and rinse (1 cc for each application) application to the gums compared to the control side. Patients were instructed to apply the rinse and gel twice a day with a cotton swab applicator to the gums and base of teeth on the same side of the mouth for one month. Chlorhexidine antiseptic, also known as chlorhexidine gluconate (CHG), was applied once daily to the opposite (control) side of the mouth.

Two groups (n=10 subjects in each group) were used to analyze reduction in periodontal pocket depth. One under the treatment with the compositions described in Table 7, and the other group using the placebo without active ingredients. Up to 13 subjects in each group were treated to allow for 30% dropout. Up to forty subjects were screened to select the 20 qualifying subjects to complete the study.

A computerized randomization scheme was used to randomize which patient will be in which group. The gingival health grading investigator and the subject were blinded as to which group received each treatment. The compositions described in Table 7 were formulated as a spray, gel, and rinse. The spray was for the sublingual application. The gel was applied as a topical application. The antimicrobial rinse was administered as a mouthwash. The control group used the identical delivery medium of the compositions of Table 7 without the active ingredients.

During the subjects' first visit to the clinical site, screening parameters were assessed/measured and recorded following gingival tissue assessment. Measurements were made at 6 sites per tooth. Probing depth, gingival index, plaque index, and intraoral photograph of gingival tissue were taken. Two postio-anterior (PA) radiographs of the quadrant were taken. From the radiographs, horizontal bone losses were calculated by measuring the distance from cementoenamel junction (CEJ) to alveola bone at three sites: mesial, distal and middle of a tooth. As post-operative care, a home care kit was provided to the two groups of patients, and patients were asked to start using the kit three days prior to the second visit. The subjects' second visit to the clinical site occurred between three days after the first visit and up to two weeks after the first visit. The visit lasted about one hour. Scaling and root plaining treatment was provided to each patient in a single quadrant. A local anesthetic effect was achieved.

The placebo group (Group 2), was administered the same carrier medium as Group 1 without active ingredients. Local anesthesia was achieved following standard of care. Full quad scaling and root plaining was rendered. In addition to the use of post-operation home care kit, patients were asked to complete HRQL (health related quality of life)/VAS (visual analog scale) patient survey on a daily basis.

During a third visit taking place four days post-operation, a HRQL (health related quality of life) patient survey on a daily basis was conducted. Following gingival tissue assessment, parameters were assessed/measured and recorded. Measurements were made at 6 sites per tooth. Probing depth, gingival index, plaque index, and intraoral photograph of gingival tissue will be taken. The third visit was about an hour long.

During a fourth visit 10 days post-operation, a HRQL (health related quality of life) patient survey was conducted on a daily basis. Following gingival tissue assessment, parameters will be assess/measured and recorded. Measurements were made at 6 sites per tooth. Probing depth, gingival index, plaque index, and intraoral photograph of gingival tissue were taken.

During the fifth visit twenty-one days post-operation, a HRQL (health related quality of life) patient survey was conducted on a daily basis. Following gingival tissue assessment, parameters will be assess/measured and recorded. Measurements were made at 6 sites per tooth. Probing depth, gingival index, plaque index, and intraoral photograph of gingival tissue were taken.

TABLE 17
Subject timeline

| | Visit | Visit | Visit | Visit | Visit |
|---|---|---|---|---|---|
| Procedures | 1 | 2 | 3 | 4 | 5 |
| Informed Consent Form | X | | | | |
| Demographics | X | | | | |
| Medical History | X | X | X | X | X |
| Evaluate eligibility and withdrawal | X | X | X | X | X |
| criteria | | | | | |
| Oral Mucosal Tissue Examination | X | X | X | X | X |
| Gingival Assessment | X | | X | X | X |
| Scaling and Root Plaining | | X | | | |
| Procedure | | | | | |
| Take Home Post Op Care use | | | X | X | X |
| Adverse Event Assessment | | | X | X | X |

After one month, the side of the mouth to which the rinse and gel were applied showed a reduction in periodontal pocket depth by 1-2 mm more than on the control side. In fact, from a periodontal pocket depth of 6-7 mm of initially, the control side had 1-2 mm reduction in the pocket depth (average) and the side administered the VEGA™ compositions described herein had average pocket reduction of 3-4 mm.

### Example 5. Anti-Inflammatory and Analgesic Effects of Arnica montana

This example analyzed the anti-inflammatory and analgesic effects of Arnica montana compared to the compositions described herein that were administered or applied in Example 1, following the surgical removal of four complete bony third molars. The overarching goal of this example was to identify remedies that could be applied both locally and systemically to reduce post-operative swelling and pain. Materials and methods were the same as those described in Example 1. Patients were selected as described in Example 1.

All patients were administered Arnica montana. The Arnica montana treatment regimen was four pellets (composition same as the spray) under the tongue four times per day. Patients were instructed to begin the Arnica montana treatment regimen three days pre-operation and to continue the regimen through the seventh day post-operation.

All surgical procedures were performed by the same surgeon, and no NSAIDS or steroids were taken for at least one month prior to the study. Visual analogue scales and consumption of post-operative analgesics were used to assess post-operative pain. Facial swelling and trismus were evaluated by measuring facial reference distances and maximum mouth opening.

Patient metrics were observed to be near baseline by the sixth-seventh day post-operation. Peak swelling was observed on the fourth post-operative day, followed by a decrease approaching baseline by the sixth to seventh post-operative day. Most patients required NORCO® (hydrocodone 5 mg/acetaminophen 325 mg) pills for post-operative pain control for the first seven days after the surgery. Table 18 shows the average number of NORCO® (hydrocodone 5 mg/acetaminophen 325 mg) pills taken by patients the first seven days after surgery.

TABLE 18
NORCO ® pill (hydrocodone 5 mg/acetaminophen 325 mg) pain management

| | Days Post-Operation | | | | | | |
|---|---|---|---|---|---|---|---|
| | DAY 1 | DAY 2 | DAY 3 | DAY 4 | DAY 5 | DAY 6 | DAY 7 |
| Average number of NORCO ® pills | 4 | 4 | 3 | 2 | 1 | 1 | 1 |
| (hydrocodone 5 mg/acetaminophen 325 mg) | | | | | | | |

Recovery following third molar extraction surgery was improved to a greater degree from treatment with compositions in Table 7 as shown by the results in Example 1 than with treatment with Arnica montana.

### Example 6. Anti-Inflammatory and Analgesic Effects of TRAUMEEL® Solution

This example evaluated the anti-inflammatory and analgesic effects of TRAUMEEL® solution containing Arnica montana radix (Mountain arnica), Calendula officinalis (Marigold), Hamamelis virginiana (Witch hazel), Millefolium (Milfoil), Belladonna (Deadly nightshade), Aconitum napellus (Monkshood), Chamomilla (Chamomile), Symphytum officinale (Comfrey), Bellis perennis (Daisy), Echinacea (Narrow-leaf coneflower), Echinacea purpurea (Purple coneflower), Hypericum perforatum (St. John's Wort), Mercurius solubilis (Hahnemann's soluble mercury), and Hepar sulphuris calcareum (calcium sulfide-made from oyster shells) compared to the compositions described herein that were administered or applied in Example 1, following the surgical removal of four complete bony third molars. The overarching goal of this example was to identify remedies that may be applied both locally and systemically to reduce post-operative swelling and pain. Materials and methods were the same as those described in Example 1. Patients were selected as described in Example 1.

All patients were administered TRAUMEEL® solution. The TRAUMEEL® solution treatment regimen was three pellets under the tongue four times per day. Patients were instructed to begin the TRAUMEEL® solution treatment regimen three days pre-operation and to continue the regimen through the seventh day post-operation.

All surgical procedures were performed by the same surgeon, and no NSAIDS or steroids were taken for at least one month prior to the analysis. Post-operative pain, facial swelling, and trismus were evaluated as described in Example 1.

Patient metrics were near baseline by the sixth-seventh day post-operation. Peak swelling occurred on the fourth post-operative day, followed by a decrease approaching baseline by the 7th post-operative day. Most patients required NORCO® pills for post-operative pain control all seven days post-operation. The average number of NORCO® pills taken by patients the first seven days after surgery is shown in Table 19.

TABLE 19
NORCO ® pain management

| | Days Post-Operation | | | | | | |
|---|---|---|---|---|---|---|---|
| | DAY 1 | DAY 2 | DAY 3 | DAY 4 | DAY 5 | DAY 6 | DAY 7 |
| Average number of NORCO ® pills | 4 | 4 | 3 | 3 | 1-2 | 1 | 1 |

When combined with prescription narcotic analgesics, TRAUMEEL® solution was not as effective for improved post-third molar extraction recovery compared to the compositions described herein as shown in Example 1.

### Example 7. Anti-Inflammatory and Analgesic Effects of Combined Arnica montana and Ibuprofen

This example evaluated the anti-inflammatory and analgesic effects of combined Arnica montana and ibuprofen treatment compared to treatment with the compositions described herein that were administered or applied in Example 1, following the surgical removal of four complete bony third molars. The overarching goal of this example was to identify remedies that may be applied both locally and systemically to reduce post-operative swelling and pain. Materials and methods were the same as those used in Example 1. Patients were selected as described in Example 1.

All surgical procedures were performed by the same surgeon, and no NSAIDS or steroids were taken for at least one month prior to the study.

All patients were administered Arnica montana. The Arnica montana treatment regimen was four pellets under the tongue four times per day. Patients were instructed to begin the Arnica montana treatment regimen three days pre-operation and to continue the regimen through the seventh day post-operation. Post-operative pain, facial swelling, and trismus were evaluated as described in Example 1.

Patient metrics were near baseline on the sixth day post-operation. Peak swelling occurred on the fourth post-operative day, followed by a decrease approaching baseline by the seventh post-operative day. Most of the patients required NORCO® for post-operative pain control all seven days post-operation. The average number of NORCO® pills for each patient for seven days post-operation is shown in Table 20.

TABLE 20
NORCO ® pain management

| | Days Post-Operation | | | | | | |
|---|---|---|---|---|---|---|---|
| | DAY 1 | DAY 2 | DAY 3 | DAY 4 | DAY 5 | DAY 6 | DAY 7 |
| Average number of NORCO ® pills | 4 | 4 | 3 | 2 | 1 | 0.5 | 0.5 |

Arnica montana and ibuprofen administered in combination was not observed to be as effective for improved post-third molar extraction recovery compared to the compositions described herein, which were administered or applied in Example 1.

### Example 8. Anti-Inflammatory and Analgesic Effects of Combined TRAUMEEL® Solution and Ibuprofen

This example evaluated the anti-inflammatory and analgesic effects of combined TRAUMEEL® solution and ibuprofen treatment compared to treatment with the compositions described herein that were administered in Example 1, following the surgical removal of four complete bony third molars. The overarching goal of this example was to identify remedies that may be applied both locally and systemically to reduce post-operative swelling and pain. Materials and methods were the same as those used in Example 1. Patients were selected as described in Example 1.

All surgical procedures were performed by the same surgeons, and no NSAIDs or steroids were taken for at least one month prior to the study.

All patients were administered TRAUMEEL® solution. The TRAUMEEL® solution treatment regimen was three pellets under the tongue four times per day. All patients additionally received: 1) 2 gm of Amoxicillin 1-hour pre-operation, and a subsequent 5 days course of 500 mg three times a day (tid); 2) NORCO® 5 mg hydrocodone/325 mg acetaminophen (15 tabs); and 3) MOTRIN® 600 mg ibuprofen.

Patients were instructed to begin the TRAUMEEL® treatment regimen three days pre-operation and to continue the treatment regimen through the seventh day post-operation. Post-operative pain, facial swelling, and trismus were evaluated as described in Example 1.

Patient metrics were near baseline on the sixth day post-operation. Peak swelling was observed on the fourth post-operative day, followed by a decrease approaching baseline by the sixth post-operative day. Most of the patients required NORCO® pills for post-operative pain control all seven days after the surgery. The average number of NORCO® pills taken by patients the first seven days after surgery is shown in Table 21.

TABLE 21
NORCO ® pills pain management

| | Days Post-Operation | | | | | | |
|---|---|---|---|---|---|---|---|
| | DAY 1 | DAY 2 | DAY 3 | DAY 4 | DAY 5 | DAY 6 | DAY 7 |
| Average number of NORCO ® pills | 4 | 4 | 3 | 2 | 1 | 0.5 | 0.5 |

TRAUMEEL® solution and ibuprofen administered in combination was not observed to be as effective for improved post-third molar extraction recovery compared to the compositions herein, which were administered or applied in Example 1.

### Example 9. Anti-Inflammatory and Analgesic Effects of Combined Arnica montana and Dexamethasone

This example evaluated the anti-inflammatory and analgesic effects of combined Arnica montana and dexamethasone treatment compared to treatment with the compositions described herein that were administered or applied in Example 1, following the surgical removal of four complete bony third molars. The overarching goal of this example was to identify remedies that may be applied both locally and systemically to reduce post-operative swelling and pain. Materials and methods used were same as those used in Example 1. Patients were selected as described in Example 1.

All surgical procedures are performed by the same surgeon, and no NSAIDS or steroids were taken for at least one month prior to the study.

All patients were administered Arnica montana. The Arnica montana regimen was four pellets under the tongue four times per day. Patients were instructed to begin the Arnica montana treatment regimen three days pre-operation, and to continue the regimen through the seventh day post-operation. All patients additionally received: 1) 2 gm of Amoxicillin 1-hour pre-operation, and a subsequent five day course of 500 mg tid; 2) NORCO® 5 mg hydrocodone/325 mg acetaminophen (15 tabs); and, 3) DECADRON® 8 mg dexamethasone IV administered once during the surgery. Post-operative pain, facial swelling, and trismus were evaluated as described in Example 1.

Patient metrics were near baseline by the sixth day post-operation. Peak swelling occurred on the fourth post-operative day, followed by a decrease approaching baseline by the seventh post-operative day. The average number of NORCO® pills taken by patients the first seven days after surgery is shown in Table 22.

TABLE 22
NORCO ® pills pain management

| | Days Post-Operation | | | | | | |
|---|---|---|---|---|---|---|---|
| | DAY 1 | DAY 2 | DAY 3 | DAY 4 | DAY 5 | DAY 6 | DAY 7 |
| Average number of NORCO ® pills | 4 | 4 | 3 | 2 | 1 | 1 | 0.5-1 |

Arnica montana and dexamethasone as a combination were not observed to be as effective for improved post-third molar extraction recovery compared to the compositions described herein, which were administered or applied in Example 1.

### Example 10. Anti-Inflammatory and Analgesic Effects of Combined TRAUMEEL® Solution and Dexamethasone

This example evaluated the anti-inflammatory and analgesic effects of combined TRAUMEEL® solution and dexamethasone treatment compared to treatment with the compositions described herein, following the surgical removal of four complete bony third molars. The overarching goal of this example was to identify remedies that may be applied both locally and systemically to reduce post-operative swelling and pain. Materials and methods were the same as those used in Example 1. Patients were selected as described in Example 1.

All surgical procedures were performed by the same surgeon, and no NSAIDS or steroids were taken for at least one month prior to the study.

All patients were administered TRAUMEEL® solution. The TRAUMEEL® solution treatment regimen was three pellets under the tongue four times per day. Patients were instructed to begin the TRAUMEEL® solution treatment regimen three days pre-operation, and to continue the treatment regimen through the seventh day post-operation. All patients additionally received: 1) 2 gm of Amoxicillin 1 hour pre-op, and a subsequent 5 day course of 500 mg tid; 2) NORCO® 5/325 mg (15 tabs); and, 3) DECADRON® 8 mg dexamethasone IV was administered once during the surgery. Post-operative pain, facial swelling, and trismus were evaluated as described in Example 1.

Patient metrics were near baseline on the sixth day post-operation. Peak swelling occurred on the fourth post-operative day, followed by a decrease approaching baseline by the sixth post-operative day. The average number of NORCO® pills taken by patients the first seven days after surgery is shown in Table 23.

TABLE 23
NORCO ® pain management

| | Days Post-Operation | | | | | | |
|---|---|---|---|---|---|---|---|
| | DAY 1 | DAY 2 | DAY 3 | DAY 4 | DAYS | DAY 6 | DAY 7 |
| Average number of NORCO ® pills | 4 | 4 | 3 | 2 | 1 | 1 | 0.5-1 |

TRAUMEEL® solution and dexamethasone as a combination was not observed to be as effective for improvement post-third molar extraction recovery compared to the compositions herein, which were administered or applied in Example 1.

### Example 11. Anti-Inflammatory and Analgesic Effects of Combined Arnica montana, Dexamethasone, and Ibuprofen

This example evaluated the anti-inflammatory and analgesic effects of administering combined Arnica montana, dexamethasone, and ibuprofen versus compositions described herein, that were administered or applied in Example 1, following the surgical removal of four complete bony third molars. The overarching goal of this example was to identify remedies that may be applied both locally and systemically to reduce post-operative swelling and pain. Materials and methods were the same as those used in Example 1. Patients were selected as described in Example 1.

All surgical procedures were performed by the same surgeon, and no NSAIDS or steroids were taken for at least one month prior to the study.

All patients were administered Arnica montana. The Arnica montana treatment regimen was four pellets under the tongue four times per day. Patients were instructed to begin the Arnica montana treatment regimen three days pre-operation and to continue the regimen through the seventh day post-operation. All patients additionally received: 1) 2 gm of Amoxicillin 1-hour pre-operation, and a subsequent 5 day course of 500 mg tid; 2) NORCO® 5/325 mg (15 tabs); 3) MOTRIN® 600 mg ibuprofen; and, 4) DECADRON® 8 mg dexamethasone IV administered once during the surgery. Post-operative pain, facial swelling, and trismus were evaluated as described in Example 1.

Patient metrics were near baseline by the fifth to sixth day post-operation. Peak swelling occurred on the third post-operative day, followed by a decrease approaching baseline by the sixth post-operative day. Most patients required NORCO® for post-operative pain control all seven days after the surgery. The average number of NORCO® pills taken by patients the first seven days after surgery is shown in Table 24.

TABLE 24
NORCO ® pain management

| | Days Post-Operation | | | | | | |
|---|---|---|---|---|---|---|---|
| | DAY 1 | DAY 2 | DAY 3 | DAY 4 | DAY 5 | DAY 6 | DAY 7 |
| Average number of NORCO ® pills | 4 | 4 | 3 | 2 | 1 | 0.5 | 0.25 |

Combined Arnica montana, dexamethasone, and ibuprofen was not as effective for improved post-third molar extraction recovery compared to the compositions described herein, which were administered or applied in Example 1.

### Example 12. Anti-Inflammatory and Analgesic Effects of Combined TRAUMEEL® Solution, Dexamethasone, and Ibuprofen

The purpose of this case report was to evaluate the anti-inflammatory and analgesic effects of combined TRAUMEEL® solution, dexamethasone, and ibuprofen treatment compared to treatment with the compositions described herein that were administered or applied in Example 1, following the surgical removal of four complete bony third molars. The overarching goal of this example was to identify remedies that may be applied locally and systemically to reduce postoperative swelling and pain. Materials and methods were the same as those used in Example 1. Patients were selected as described in Example 1.

All surgical procedures were performed by the same surgeon, and no NSAIDS or steroids were taken for at least one month prior to the study.

All patients were administered TRAUMEEL® solution. The TRAUMEEL® solution treatment regimen was three pellets under the tongue four times per day. Patients were instructed to begin the TRAUMEEL® solution treatment regimen three days pre-operation, and to continue the treatment regimen through the seventh day post-operative. All patients additionally received: 1) 2 gm of Amoxicillin 1-hour pre-operation and a subsequent 5 day course of 500 mg tid; 2) NORCO® 5/325 mg (15 tabs); 3) MOTRIN® 600 mg ibuprofen; and 4) DECADRON® 8 mg dexamethasone IV administered once during the surgery. Post-operative pain, facial swelling, and trismus were evaluated as described in Example 1.

Patients metrics were near baseline by the sixth day post-operation. Peak swelling occurred on the third post-operative day, followed by a decrease approaching baseline by the seventh post-operative day. Most patients required NORCO® for post-operative pain control all seven days after the surgery. The average number of NORCO® pills taken by patients the first seven days after surgery is shown in Table 25.

TABLE 25
NORCO ® pill pain management

| | Days Post-Operation | | | | | | |
|---|---|---|---|---|---|---|---|
| | DAY 1 | DAY 2 | DAY 3 | DAY 4 | DAY 5 | DAY 6 | DAY 7 |
| Average number of NORCO ® pills | 4 | 4 | 3 | 2 | 1 | 0.5 | 0.25 |

TRAUMEEL® solution, dexamethasone, and ibuprofen as a combination was not observed to be as effective for improved post-third molar extraction recovery compared to the compositions described herein, which were administered or applied in Example 1.

### Example 13. Comparison of Results from Examples 1 and 5-12

Table 26 summarizes the data from Examples 1 and 5-11, showing the average number of NORCO® pills taken by patients the first seven days after surgery, across treatment regimens. Fewer NORCO® pills were required for pain-management in patients treated with the compositions in Example 1 compared to those treated with homeopathics, steroids, and/or NSAIDS described in Examples 5-11. As shown in Table 26, the number of NORCO® pills required was markedly reduced by day three in patients administering or applying the compositions in Example 1, and recovery from surgery was accelerated. The time to peak swelling was also accelerated in patients administered or application of the compositions described herein.

TABLE 26
Number of NORCO ® pills taken days 1-7 post-operation

| | | Day 1 | Day 2 | Day 3 | Day 4 | Day 5 | Day 6 | Day 7 | Day of peak | Day of full |
|---|---|---|---|---|---|---|---|---|---|---|
| Treatment | Example | post-op | post-op | post-op | post-op | post-op | post-op | post-op | swelling | recovery |
| V/N | 1 | 3.5 | 2.3 | 0.8 | 0.6 | 0.5 | 0.4 | 0.25 | 1-2 | 3-4 |
| A/N | 5 | 4.0 | 4.0 | 3.0 | 2.0 | 1.0 | 1.0 | 0.5-1.0 | 4 | 6-7 |
| T/N | 6 | 4.0 | 4.0 | 3.0 | 2.0 | 1.0 | 1.0 | 0.5-1.0 | 4 | 6-7 |
| A/N/M | 7 | 4.0 | 4.0 | 3.0 | 2.0 | 1.0 | 0.5 | 0.5 | 4 | 6-7 |
| T/N/M | 8 | 4.0 | 4.0 | 3.0 | 2.0 | 1.0 | 0.5 | 0.5 | 4 | 6 |
| A/N/D | 9 | 4.0 | 4.0 | 3.0 | 2.0 | 1.0 | 1.0 | 0.5-1.0 | 4 | 6-7 |
| T/N/D | 10 | 4.0 | 4.0 | 3.0 | 2.0 | 1.0 | 1.0 | 0.5-1.0 | 4 | 6-7 |
| A/N/M/D | 11 | 4.0 | 4.0 | 3.0 | 2.0 | 1.0 | 0.5 | 0.5 | 3 | 5-6 |
| T/N/M/D | 12 | 4.0 | 4.0 | 3.0 | 2.0 | 1.0 | 0.5 | 0.5 | 3 | 5-6 |

V = VEGA ™ compositions (e.g., drops, gel, and rinse from Example 1),
A = Arnica montana,
T = TRAUMEEL ® solution,
D = DECADRON ® dexamethasone IV,
N = NORCO ® pill (Hydrocodone 5/acetaminophen 325 mg),
M = MOTRIN ® 600 mg ibuprofen,

Fewer NORCO® pills were required for pain management by patients who received the compositions administered or applied in Example 1 compared to Example 5, wherein patients were administered Arnica montana, and Example 6, where patients were administered TRAUMEEL® solution. The time to peak swelling was also reduced in the patients receiving the compositions described herein, as shown in Table 26.

Fewer days were required for full post-operative recovery by patients receiving the compositions administered or applied in Example 1 compared to those receiving other treatments. Overall, results herein indicate that administration or application of the compositions described herein is superior to alternate treatment regimens for reduced inflammation and pain control. The compositions administered or applied in Example 1 show a synergistic effect over single substance, for example, Arnica montana.

### Example 14. Reducing Opioid Prescription and Use Levels

To reduce opioid prescription and use levels, a three-year retrospective analysis was performed. The purpose of the study was to evaluate “strong” opioid prescription and use at a single-surgeon oral surgery practice (hereinafter referred to as “the practice”) following the introduction of a protocol designed to offer alternatives to opioids for post-operative pain management following oral surgeries. For this study, “strong” opioid prescription/use was defined as anything greater, or with a heavier/stronger pain relief profile, than 30 mg of Codeine. The hypothesis was that introduction of the protocol, described in further detail below, would significantly reduce opioid prescription and use levels.

The study population was composed of all adult patients who had been seen at the practice over a three-year period and had undergone any of the following qualified procedures: removal of impacted wisdom teeth, complex guided bone restoration (GBR)/bone reconstructions including on-lay and inter-positional bone grafting, lateral sinus lifts, dental implants including full arch dental implants rehabilitation with immediate occlusal loading.

These procedures were used as study inclusion criteria and are routinely accompanied by prescription of ‘strong opioids’ for post-operative pain management. Procedures such as biopsies, routine extractions, cosmetic procedures as well as orthognathic surgeries were excluded from inclusion in the study as these procedures routinely do not require prescription of opioid pain medications, and/or monitoring of pain management in a hospital setting.

Patients who had undergone surgical procedures within the designated three-year period at the practice were obtained from the office software database (OMSVISION®). Data from the same time frame available through Illinois Monitoring Service was analyzed for patients seen at the practice, each of whom had obtained and filled “strong” opioid (hydrocodone and oxycodone) prescriptions for qualified procedures and were routinely given prescriptions stronger than 30 mg Codeine or “strong” opioids. Proportions of patients filling “strong” opioid prescriptions and associated 95% confidence intervals were used to quantify “strong” opioid prescriptions filled by patients over the three-year interval.

The study protocol was designed to optimize preemptive analgesia using a combination of NSAIDs, acetaminophen, and the compositions described in Table 7 introduced as a kit, also referred to as VEGA™ Oral Recovery Kit (hereinafter “the kit”), for post-operative pain management. Pre-emptive analgesia was introduced either pre-operatively or immediately post-operatively, when intraoperative local anesthetic remains effective. Patients began use of the kit three days pre-operation, and application continued for seven days post-operation.

The composition described herein formulated as a gel shown in Example 1 was applied on the surgical site immediately post-operation. Patients were also administered 1000 mg of acetaminophen and 400 mg of MOTRIN® ibuprofen immediately post-operation. The combination was administered or applied up to four times per day, not to exceed the allowable daily dose. The annual number of “strong” opioid prescriptions, total qualifying cases, proportion of procedures leading to filled “strong” opioid prescriptions, and corresponding confidence intervals for patients included in the present study are shown in Table 27. The primary outcome variable was the proportion of procedures leading to filled “strong” opioid prescriptions. The predictor variable was time (year).

TABLE 27
Proportions of procedures leading to filled “strong” opioid prescriptions

| Year | 1 | 2 | 3 |
|---|---|---|---|
| Number of | 1184 | 899 | 387 |
| Hydrocodone/Oxycodone prescriptions | | | |
| Total qualified cases performed that include: impacted wisdom | 2016 | 2005 | 2034 |
| teeth removal, GBR/Bone augmentation/reconstruction, sinus lift, | | | |
| dental implants, Full arch reconstruction (All on 4/5/6) | | | |
| % of patients undergoing procedures leading to filled Rx's | 58.7% | 44.8% | 19.0% |
| 95% Confidence intervals | (56.7%-60.5%) | (42.3%-46.6%) | (17.7-20.8%) |

In the first year of the study, 2016 adult patients underwent qualified surgical procedures. Total proportions of patients filling strong opioid prescriptions decreased over the three years of the study: 59% (95% confidence interval 57%-61%) in the first year of the study to 19% (95% confidence interval 18%-21%) in the third year of the study, a relative risk of 0.32. The number of qualified surgical procedures performed remained relatively stable. Therefore, a three-fold reduction in the number of “strong” opioid (Hydrocodone/Oxycodone) prescriptions occurred over three years of application of the protocol described in this example.

Gender and age demographics for patients filling “strong” opioid prescriptions are shown in Table 28. Patient ages ranged from 15-85 years.

TABLE 28
Gender and age demographics for patients filling
“strong” opioid prescriptions

| | Year | | |
|---|---|---|---|
| | 1 | 2 | 3 |
| | (n = 1184) | (n = 899) | (n = 387) |
| Gender (% male) | 46% | 46% | 50% |
| Age (mean) | 45 | 44 | 38 |

Patients who filled “strong” opioid prescriptions were similar in gender distribution and average age between year 1 and year 2. By comparison, patients included in the study in year 3 were slightly older on average, with a higher representation of males.

Distributions of qualifying oral surgery procedures were similar through the three-year time frame as shown in Table 29.

TABLE 29
Distributions of qualifying oral surgery procedures

| Procedure | 1 (n = 2016) | | 2 (n = 2005) | | 3 (n = 2034) | |
|---|---|---|---|---|---|---|
| Impacted teeth removal | 443 | 22% | 568 | 28% | 498 | 24% |
| Bone augmentation/ | 787 | 39% | 708 | 35% | 720 | 35% |
| reconstruction | | | | | | |
| Sinus life | 47 | 2% | 60 | 3% | 109 | 5% |
| Dental implants | 706 | 35% | 626 | 31% | 656 | 32% |
| Full arch reconstruction | 33 | 2% | 43 | 2% | 51 | 3% |
| (All on 5/6) | | | | | | |

Therefore, the protocol described in this example, which included combined administration or application of NSAIDs, acetaminophen, and the VEGA™ compositions described herein, decreased strong opioid use for pain management following oral surgery procedures. All procedures in the present study were performed by a single surgeon.

One assumption of the present study was that post-operative pain was well-managed as patients did not request stronger pain medications. Patients can be given Health Related Quality of Life Questionnaire (HRQL) to further evaluate their pain control (See, Shugars et al. JOMS. 1996, 54: 1402-1408, which is hereby incorporated by reference in its entirety).

### Example 15. Treatment of Mucositis and Thrush Using the VEGA™ Oral Recovery Kit

A patient with a long-standing bilateral buccal mucositis was treated with a VEGA™ Oral Recovery Kit according to the protocol described in the previous Examples. One month of treatment with the gel, rinse, and spray resulted in complete resolution of the lesion on the left side, and over 80% resolution on the right side. Therefore, use of the VEGA™ Oral Recovery Kit was observed to significantly accelerate healing from buccal mucositis.

### Example 16. Treatment of Oral Lichen Planus with a Eel and Rinse

Two middle-aged female patients complained of a burning sensation in the mouth and gum sensitivity. After clinical evaluation, a differential diagnosis of lichen planus/esophageal lichen planus (ELP) was considered. A decision was made to perform an incisional biopsy.

Patients were placed on an oral gel and rinse course (from Table 7) three times daily after the incisional biopsy. The patients were seen for follow-up and re-evaluation one-week post-incisional biopsy.

At the one-week follow-up appointment, clinical evaluation showed significant resolution of the lesions. The patients' symptoms of burning had resolved. Therefore, a one-week course of the gel and rinse was observed to resolve acute presentation of lichen planus/esophageal lichen planus (ELP), including clinical symptoms of mouth burning.

### Example 17. Reduction of Anaerobic Bacteria Using Oral Rinse

A BREATHOMETER™ Mint device was used to measure the anaerobic bacterial load in parts per billion (ppb) in the mouth. The device captures bacteria biofilm gases in the mouth, such as hydrogen sulfite, methyl mercaptan, and hydrogen disulfide.

After rinsing with 10 cc of oral rinse described in Table 7, BREATHOMETER™ Mint device measurements were obtained at 5, 10, 30 minutes and hourly for the next 3 hours. A single application of an oral rinse was observed to significantly reduce anaerobic bacterial load for over 3 hours. The baseline after brushing and rinsing was 96 ppb. After 30 minutes and up to 3 hours the load dropped to 50 ppb.

### Example 18. Treatment of Focal Mucositis/Mucinosis Using Oral Gel

A 73-year old female patient with a previous history of squamous cell carcinoma (SCCA), who had undergone therapy transplant treatment, presented for evaluation of a retromolar lesion. An incisional biopsy was performed to confirm the diagnosis of a focal mucinosis.

A cotton swab applicator full of the oral gel from Table 7 was applied three times per day. Complete resolution of the lesion was observed at the one-month follow-up appointment. Therefore, the oral gel was observed to completely resolve the lesion associated with mucositis/mucinosis in the retromolar pad area.

### Example 19. Analysis of Anti-Inflammatory and Analgesic Effects for Modified Formulation

The anti-inflammatory and analgesic effects of compositions described herein formulated as a spray/drops, a gel, and a rinse were analyzed after the surgical removal of four complete bony third molars. The formulations of the compositions administered or applied to the patients are shown in Table 4 (Kit 1).

A group of patients requiring removal of third molars were selected for surgery. Administration or application of the composition formulations as described in Table 1 was initiated three days before the surgery and continued for 7 days after the surgery. Patients gargled with the rinse (10-15 cc) three times daily for 2-3 minutes, and then spit out the rinse.

Patients were sublingually administered 15 drops (spray formulation) containing about 0.07 cc each four times daily. Patients were instructed not to swallow for about one minute. The gel (about 0.02 cc) was applied to the gum surrounding the tooth extraction site three times daily using a cotton swab applicator. Patients did not rinse or eat for 20 minutes after application, allowing for absorption of the gel.

Patients were administered four Amoxicillin (500 mg/tablet) or Clindamycin (150 mg/tablet) tablets one hour before surgery. Patients had the option to take the Amoxicillin or Clindamycin tablets with water.

Facial swelling and trismus were evaluated by measuring facial reference distances and maximum mouth opening size, respectively. Facial swelling and trismus were measured on post-operative days 2, 4, and 7.

Mouth opening size, which is defined as the maximum distance between upper and lower central incisors, was measured by a ruler to the nearest mm, and the pre-operation (pre-op) value was recorded as the baseline. Trismus, which is a reduced opening of the jaws, was measured the same way on days 2, 4 and 7 post-operatively. Table 30 shows the average measurements obtained during assessment of trismus in the patient.

TABLE 30
Trismus/Mouth Opening

| | Days Post-Operation | | | |
|---|---|---|---|---|
| | Pre-Operation | | | |
| Measurements | (baseline) | DAY 2 | DAY 4 | Day 7 |
| mm | 49 | 28.51 | 33.12 | 43.5 |
| % trismus | — | 58% | 67.6% | 88.8% |

Results in Table 30, when compared with results in Table 12 from Example 1, show improved Trismus/Mouth Opening results from treatment with Kit 1 (Table 4) compared to treatment with Kit 4 (Table 7). Treatment with Kit 1 showed 58% (Day 2), 67.6% (Day 4) and 88.8% (Day 3) improvement in Trismus, while treatment with Kit 4 showed 46% (Day 2), 61% (Day 4) and 81% (Day 3) improvement in Trismus.

Facial swelling was measured by a tape measuring method. Four measurements were made between 6 reference points: tragus, lateral canthus of eye, pogonion, ala, gonion and the corner of the mouth. The pre-operation (pre-op) sum of the measurements was recorded. Table 31 shows the measurements of swelling in the patients.

TABLE 31
Swelling

| | Days Post-Operation | | | |
|---|---|---|---|---|
| | Pre-Operation | | | |
| Measurements | (baseline) | DAY 2 | DAY 4 | Day 7 |
| Sum of measurements | 918.61 | 938.52 | 924.58 | 919.1 |
| [mm] | | | | |
| % of baseline | | 102.2% | 100.6% | 100.5% |
| % change from previous | | 2.2% | 0.6% | 0.05% |
| measurement | | | | |

Results in Table 31, when compared with results in Table 13 from Example 1, show that improvements in swelling which result from treatment with Kit 4 (Table 7) are maintained by treatment with Kit 1 (Table 4). There was also a slight improvement in swelling over the first 2-3 days of treatment when using Kit 1.

Post-operative pain was assessed using a visual analogue scale (VAS) anchored by verbal descriptors. Specifically, post-operative pain was rated daily for 7 days using a 100-point VAS anchored by the verbal descriptors “no pain” (0) and “very severe pain” (100). Pain levels between about 10 to about 30 are indicative of a range of mild pain, pain levels between about 40 to about 60 are indicative of a range of moderate pain, and pain levels between about 70 to 100 are indicative of a range of severe pain. VAS daily pain measurements of post-operative pain are shown in Table 32.

TABLE 32
Daily pain measurements

| | Days Post-Operation | | | | | | |
|---|---|---|---|---|---|---|---|
| | DAY 1 | DAY 2 | DAY 3 | DAY 4 | DAY 5 | DAY 6 | DAY 7 |
| Severity (Patient) | 61.5 | 43.1 | 39.8 | 30.8 | 25.4 | 17 | 5 |
| Severity (Clinician) | — | 52.1 | — | 32.8 | — | — | 10 |
| Intensity (Patient) | 52.7 | 33.2 | 30.1 | 25.4 | 15.1 | 12.8 | 5 |
| Intensity (Clinician) | — | 41.8 | — | 22.5 | — | — | 5 |

Results in Table 32, when compared with results in Table 14 from Example 1, show improved VAS daily pain measurements of post-operative pain from treatment with Kit 1 (Table 4) compared to treatment with Kit 4 (Table 7). On average, treatment with Kit 1 resulted in VAS Pain Severity assessments which were 15-20% lower when compared to results from treatment with Kit 4. On average, treatment with Kit 1 resulted in VAS Pain Intensity assessments which were 10-18% lower for Days 1-4, and as much as 30-40% lower for Days 5-7, when compared to results from treatment with Kit 4.

Post-operative pain was also assessed using patient self-reports on the number of analgesic tablets required for post-operative pain relief. Patients received 15 pills in tablet form of NORCO® (hydrocodone 5 mg/acetaminophen 325 mg) to be used if needed. Table 33 shows the average number of NORCO® (hydrocodone 5 mg/acetaminophen 325 mg) pills taken per patient for the first seven days post-operation.

TABLE 33
NORCO ® pill pain management

| | Days Post-Operation | | | | | | |
|---|---|---|---|---|---|---|---|
| | DAY 1 | DAY 2 | DAY 3 | DAY 4 | DAYS | DAY 6 | DAY 7 |
| Average number of NORCO ® pills | 2 | 1.5 | 0.2 | 0.6 | 0.5 | 0.3 | 0.1 |

Results in Table 33, when compared with results in Table 15 from Example 1, show improved NORCO reduction and post-operative pain management from treatment with Kit 1 (Table 4) compared to treatment with Kit 4 (Table 7). On average, treatment with Kit 1 resulted in the following reductions of NORCO pills when compared to results from treatment with Kit 4: 1.5 fewer pills for Day 1; 0.8 fewer pills for Day 2; and 0.3 fewer pills for Day 3.

A comparison of the results in Example 19 with results presented in Example 1 show an unexpected improvement in anti-inflammatory and analgesic effects from treatment of patient with Kit 1 compared to treatment with Kit 4.

### Example 20. Tonsillar Spray

A tonsillar spray having anti-inflammatory, analgesic and antimicrobial properties was studied for use in treatments before and after tonsillar/uvular surgery. The study included exfoliation properties for tonsillar stones.

The tonsillar spray composition included the formulation shown in Table 8.

Ten (10) patients were selected, each having been diagnosed with tonsillar stones associated with tonsillar/uvular surgery. At initial examination (prior to treatment), five (5) patients had level 3 tonsillar stones, three (3) patients had level 2 tonsillar stones, and two (2) patients had level 1 tonsillar stones.

Each patient was treated with a combination of the tonsillar spray and oral rinse from Table 8 (4 sprays followed by 10 cc rinse/expectorate), four (4) times per-day for ten (10) days.

All ten patients were re-evaluated on Day #7. Clinical exams detected no tonsillar stones in the 3 patients initially having level 2 tonsillar stones, the 2 patients initially having level 1 tonsillar stones, and 3 of the patients initially having level 3 tonsillar stones. The tonsillar stones in the remaining two patients (initially level 3) had been reduced to level 1 tonsillar stones. On Day #10, no tonsillar stones were detected in any of the ten patients.

### Example 21. Advanced Healing & Protecting (AHP) Oral Rinse

An Advanced Healing & Protecting (AHP) oral rinse having radioprotective, soothing, anti-inflammatory and antimicrobial properties was studied for use with chemotherapy or radiation therapy for Head & Neck (H&N) cancer patients, including the possible decrease in certain side-effects from the radiation/chemo treatment.

The AHP oral rinse composition included the formulation shown in Table 10.

Twenty (20) patients were selected, each undergoing H&N chemo and/or radiation therapy for intra-oral squamous cell carcinoma. All 20 patients received Baseline clinical assessments for the following: (i) Dry Mouth Visual Analog Scale (DMVAS, scale of 0-10, with 10 being the worst); and (ii) Baseline number of Ulcers (at least 5 mm).

Ten (10) patients were then treated daily with the oral gel and AHP oral rinse of Table 10. Ten (10) patients were treated with placebo rinse and gel.

Clinical exams at 10 days provided the results shown in Table 34.

TABLE 34
Results from treatment with Kit 7

| | | DMVAS | DMVAS | Ulcers | Ulcers |
|---|---|---|---|---|---|
| Patient | Rinse | (Baseline) | (Day 10) | (Baseline) | (Day 10) |
| 1 | AHP/Gel | 8 | 4 | 5 | 2 |
| 2 | AHP/Gel | 7 | 3 | 7 | 2 |
| 3 | AHP/Gel | 9 | 4 | 3 | 1 |
| 4 | AHP/Gel | 8 | — | 2 | 0 |
| 5 | AHP/Gel | 9 | 3 | 4 | 2 |
| 6 | AHP/Gel | 7 | 4 | 3 | 1 |
| 7 | AHP/Gel | 7 | 3 | 6 | 3 |
| 8 | AHP/Gel | 6 | 6 | 2 | 1 |
| 9 | AHP/Gel | 6 | 5 | 3 | 2 |
| 10 | AHP/Gel | 9 | 2 | 5 | 2 |
| AVG | AHP/Gel | 7.6 | 3.8 | 4.0 | 1.6 |
| 11 | Placebo | 5 | 5 | 2 | 2 |
| 12 | Placebo | 6 | 4 | 2 | 2 |
| 13 | Placebo | 7 | 7 | 7 | 5 |
| 14 | Placebo | 7 | 8 | 4 | 3 |
| 15 | Placebo | 9 | 7 | 3 | 3 |
| 16 | Placebo | 9 | 9 | 5 | 4 |
| 17 | Placebo | 8 | 8 | 5 | 3 |
| 18 | Placebo | 6 | 5 | 7 | 7 |
| 19 | Placebo | 5 | 5 | 3 | 2 |
| 20 | Placebo | 7 | 7 | 3 | 3 |
| AVG | Placebo | 6.9 | 6.5 | 4.1 | 3.4 |

Patients receiving the oral gel and AHP oral rinse of Table 10 had a 60% reduction in ulcers, compared to a 17% reduction in the placebo group. Patients receiving the oral gel and AHP oral rinse of Table 10 also had a 50% reduction in the Dry Mouth Visual Analog Scale, compared to a 6% reduction in the placebo group.

### Example 22. Toothpaste

A toothpaste was provided which had the following properties: minimally foaming, all-natural, encourages faster healing, reduces bleeding, freshens breath, good smell, soothing taste, non-gritty, silky, antimicrobial properties, Tartar control, tooth whitening, reduction of tooth sensitivity, and re-mineralizing. The toothpaste was studied for use in treating periodontal disease, including improvements in periodontal pocket depth and reduced gum bleeding.

The toothpaste composition included the formulation shown in Table 9.

Ten (10) patients were selected, each having moderate periodontal disease as measured by: (i) Periodontal Pockets (PP) of 5-6 mm for representative teeth (buccal and lingual); and (ii) Bleeding on Probing (BOP) of representative gums (buccal and lingual), based on clinician's assessment. Each patient was treated with the toothpaste from Table 9 twice (2× times) per day for one month. All ten patients were re-evaluated after one month (periodontal pockets measurement and gum probing).

Clinical results at Baseline (pretreatment) and at 10 days are shown in Table 35.

TABLE 35
Results from treatment with Kit 6

| | Buccal PP | Lingual PP | Buccal PP | Lingual PP | BOP | BOP |
|---|---|---|---|---|---|---|
| Patient | (Baseline, mm) | (Baseline, mm) | (1 month, mm) | (1 month, mm) | (Baseline) | (1 month) |
| 1 | 5/5/6 | 4/4/3 | 4/4/3 | 3/3/3 | Y/Y | N/N |
| 2 | 6/6/5 | 5/4/5 | 3/4/3 | 3/2/3 | Y/Y | N/N |
| 3 | 6/7/6 | 5/6/5 | 3/4/5 | 3/3/4 | Y/Y | N/N |
| 4 | 4/5/5 | 6/5/4 | 3/3/3 | 3/3/3 | N/Y | N/N |
| 5 | 5/6/5 | 5/5/4 | 3/4/3 | 3/3/4 | Y/Y | N/N |
| 6 | 4/5/5 | 6/5/6 | 3/3/3 | 3/4/3 | Y/Y | N/N |
| 7 | 6/5/5 | 6/6/7 | 4/5/5 | 6/5/5 | Y/Y | Y/Y |
| 8 | 6/5/5 | 5/7/5 | 5/5/4 | 4/5/5 | Y/Y | N/N |
| 9 | 6/6/7 | 4/4/5 | 5/5/6 | 3/4/3 | Y/N | N/N |
| 10 | 6/5/5 | 6/6/7 | 3/3/4 | 3/4/4 | Y/Y | N/N |
| AVG | 5.43 | 5.17 | 3.83 | 3.57 | Y/Y | N/N |

Clinical exams showed that patients had an average Periodontal Pocket improvement of about 30% (1.6 mm improvement on average). Nine out of ten patients also showed significantly reduced Bleeding on Probing.

### Example 23. Topical Healing Balm

A topical balm was provided which had the following properties: Cutaneous application, decreased inflammation, encourage healing, decrease pain, and decreased scar formation. The topical balm was studied for use with topical laser procedures and in treating general skin burns, including the encouragement of skin healing, pain management, reepithalization, preventing scar formation and tissue contraction, and increased Second Intention healing after skin trauma.

The Topical Healing Balm composition included the formulation shown in Table 11.

Twenty (20) patients were selected, each receiving partially or fully ablative CO₂ laser treatment. Ten (10) patients were treated regularly with the Topical Healing Balm (THB) of Table 11. Ten (10) patients were treated with Aquaphor Healing Ointment® as a control. All 20 patients received an initial clinical assessments on Day 2 of treatment for pain using a Visual Analogue Scale (VAS, 0-100). Another VAS clinical assessment for pain was completed on Day 10 of treatment. The results are shown in Table 36.

TABLE 36
Results from treatment with Kit 8

| | | VAS | VAS |
|---|---|---|---|
| Patient | Treatment | (Day 2) | (Day 10) |
| 1 | THB | 60 | 10 |
| 2 | THB | 55 | 5 |
| 3 | THB | 65 | 20 |
| 4 | THB | 45 | 10 |
| 5 | THB | 60 | 20 |
| 6 | THB | 30 | 10 |
| 7 | THB | 65 | 5 |
| 8 | THB | 65 | 10 |
| 9 | THB | 55 | 20 |
| 10 | THB | 50 | 20 |
| AVG | THB | 55 | 13 |
| 11 | Aquaphor | 80 | 45 |
| 12 | Aquaphor | 85 | 30 |
| 13 | Aquaphor | 65 | 30 |
| 14 | Aquaphor | 60 | 40 |
| 15 | Aquaphor | 85 | 45 |
| 16 | Aquaphor | 89 | 40 |
| 17 | Aquaphor | 70 | 20 |
| 18 | Aquaphor | 80 | 20 |
| 19 | Aquaphor | 85 | 25 |
| 20 | Aquaphor | 60 | 35 |
| AVG | Aquaphor | 76 | 33 |

After 2 days and 10 days, clinical exams showed that patients treated with the Topical Healing Balm of Table 11 demonstrates accelerated healing when compared to control patients (Aquaphor), according to VAS patient assessments. Patients treated with the Topical Healing Balm of Table 11 had a VAS pain level on Day 2 which was 21 points lower (on average) when compared to Aquaphor control patients, and a VAS pain level on Day 10 which was 20 points lower (on average) when compared to Aquaphor control patients.

1. A composition comprising: an attenuation of Arnica montana; an attenuation of Calendula officinalis; an attenuation of Chamomilla; an attenuation of Ignatia amara; an attenuation of Ledum palustre; an attenuation of Silicea; and at least one of an attenuation of Echinacea purpurea or an attenuation of Echinacea angustifolia.

2. The composition of claim 1, wherein the composition further comprises at least one ingredient selected from: an attenuation of Azadirachta indica; an attenuation of Hepar sulphuris calcaneum; an attenuation of Hypericum; an attenuation of Mercurius vivus; an attenuation of Ruta graveolens; an attenuation of Plantago major; an attenuation of Romains officinalis; an attenuation of Origanum vulgare L; an attenuation of Thymus vulgaris L; an attenuation of Lavandula angustifolia/officinalis; an attenuation of Salvia officinalis; an attenuation of Melissa officinalis; an attenuation of Cuminum cyminum; an attenuation of Petrosehnum crispum; an attenuation of Calendula officinalis; an attenuation of Tagetes erecta; an attenuation of Boswellia sacra; an attenuation of Sambucus nigra; an attenuation of Copaifera langsdorfii; an attenuation of Curcuma longa; an attenuation of Allium sativu; an attenuation of Punica granatum; an attenuation of Euterpe oleracea; an attenuation of Sophora flavescens; an attenuation of Rheum rhabarbarum; an attenuation of Fagopyrum esculentum; an attenuation of Camellia sinensis; an attenuation of Coptis teeta; an attenuation of Hydrastis canadensis; an attenuation of Mahonia aquifolium; an attenuation of Phellodendron amurense; an attenuation of Berberis vulgaris; an attenuation of Xanthorhiza simplicissima; an attenuation of Lonicera ceprifoliu; an attenuation of Vaccinium macrocarpon; an attenuation of Cinnamomum zeylanicum Nees; an attenuation of Cinnamomum verum; an attenuation of Vitis vinifera; an attenuation of Terminalia Billerica; an attenuation of Pinus pinaster; an attenuation of Albizia lebbek; an attenuation of Melia azadirachta; an attenuation of Salvadora persica; an attenuation of Paullinia cupana; an attenuation of Piper betle; an attenuation of Syzygium aromaticum; an attenuation of Commiphora myrrha; an attenuation of Juglans regia; an attenuation of Scutellaria baicalensis; an attenuation of Magnolia officinalis; an attenuation of Origanum vulgare; an attenuation of Origanum onites; an attenuation of Origanum majorana; an attenuation of Origanum heracleoticum; an attenuation of Thymus citriodorus; an attenuation of Thymus pulegioides; an attenuation of Thymus x herba-barona; an attenuation of Thymus serpyllum; an attenuation of Lavandula stoechas; an attenuation of Lavandula dentate; an attenuation of Lavandula x intermedia; an attenuation of Lavandula multifida; an attenuation of Salvia divinorum; an attenuation of Salvia apiana; an attenuation of Calendula arvensis; an attenuation of Calendula maderensis; an attenuation of Tagetes minuta; an attenuation of Tagetes patula; an attenuation of Boswellia frereana; an attenuation of Boswellia serrata; an attenuation of Boswellia papyrifera; an attenuation of Sambucus melanocarpa; an attenuation of Sambucus racemosa; an attenuation of Rheum rhaponticum; an attenuation of Citrullus colocynthis; and an attenuation of Cucumis sativus.

3. (canceled)

4. (canceled)

5. (canceled)

6. (canceled)

7. (canceled)

8. (canceled)

9. (canceled)

10. (canceled)

11. The composition of claim 1, wherein the composition further comprises at least one of: an allopathic medicine, a vitamin, a mineral, or an amino acid.

12. (canceled)

13. The composition of claim 11, wherein the mineral is selected from: Antimony, Barium, Beryllium, Bismuth, Boron, Bromine, Cadmium, Calcium, Carbonate, Cerium, Cesium, Chloride, Chromium, Cobalt, Copper, Dysprosium, Erbium, Europium, Gadolinium, Gallium, Germanium, Gold, Hafnium, Holmium, Indium, Iodine, Iridium, Iron, Lanthanum, Lead, Lithium, Lutetium, Magnesium, Manganese, Molybdenum, Neodymium, Nickel, Niobium, Osmium, Palladium, Propolis, Phosphorus, Platinum, Potassium, Praseodymium, Rhenium, Rhodium, Rubidium, Ruthenium, Samarium, Scandium, Selenium, Silicon, Silver, Sodium, Strontium, Sulfate/Sulfur, Tantalum, Tellurium, Terbium, Thallium, Thorium, Thulium, Tin, Titanium, Tungsten, Vanadium, Ytterbium, Yttrium, Zinc, and Zirconium.

14. The composition of claim 1, wherein the composition comprises an excipient selected from: viscosity modifying agents, buffers, antioxidants, emulsifying agents, absorbents, antiacne agents, antiperspirants, anticaking agents, antifoaming agents, antimicrobial agents, anti-infective agents, antioxidants, antidandruff agents, astringents, binders, buffers, biological additives, buffering agents, bulking agents, chelating agents, chemical additives, coupling agents, conditioners, colorants, cosmetic astringents, cosmetic biocides, denaturants, drug astringents, detergents, dispersants, external analgesics, film formers, foaming agents, fragrance components, flavoring agents humectants, keratolytics, opacifying agents, pH adjusters, preservatives, propellants, proteins, retinoids, reducing agents, sequestrants, skin bleaching agents, skin-conditioning agents (humectants, miscellaneous, and occlusive), skin soothing agents, skin healing agents, softeners, solubilizing agents, lubricants, penetrants, plasticizers, solvents and co-solvents, sun screening additives, sweeteners, salts, essential oils, and vitamins.

15. (canceled)

16. (canceled)

17. (canceled)

18. (canceled)

19. The composition of claim 1, wherein the composition is formulated as a gel, a rinse, a spray, a pellet, a pill, or a drop.

20. (canceled)

21. (canceled)

22. (canceled)

23. The composition of claim 1, wherein the composition comprises the attenuation of Arnica montana at a dilution of 3X, 6X, or 18X.

24. The composition of claim 1, wherein the composition comprises the attenuation of Calendula officinalis at a dilution of 3X.

25. The composition of claim 1, wherein the composition comprises the attenuation of Chamomilla at a dilution of 5X.

26. The composition of claim 1, wherein the composition comprises the attenuation of Echinacea purpurea at a dilution of 3X.

27. (canceled)

28. (canceled)

29. The composition of claim 1, wherein the composition comprises the attenuation of Ignatia amara at a dilution of 30X.

30. The composition of claim 1, wherein the composition comprises the attenuation of Ledum palustre at a dilution of 6X.

31. (canceled)

32. (canceled)

33. (canceled)

34. (canceled)

35. The composition of claim 1, wherein the composition comprises the attenuation of Silicea at a dilution of 8X.

36. The composition of claim 1, wherein the composition comprises at least one ingredient selected from: allantoin, citric acid, glycerin, hydroxyethyl cellulose, peppermint oil, potassium sorbate, polysorbate 20, propolis, purified water, sodium benzoate, and xylitol.

37. The composition of claim 1, wherein the composition comprises a bupivacaine liposome injectable suspension.

38. (canceled)

39. A kit comprising a first composition formulated as a spray or drops, a second composition formulated as a gel, a third composition formulated as a rinse, and at least one container; wherein
a. the first and second compositions comprise;
i. an attenuation of Hepar sulphuris calcaneum at a dilution of 8X,
ii. an attenuation of Calendula officinalis at a dilution of 3X,
iii. an attenuation of Chamomilla at a dilution of 5X,
iv. an attenuation of Ledum palustre at a dilution of 6X,
v. an attenuation of Echinacea angustifolia at a dilution of 3X or an attenuation of Echinacea purpurea at a dilution of 3X,
vi. an attenuation of Ruta graveolens at a dilution of 12X,
vii. an attenuation of Arnica montana at a dilution of 3X, 6X, or 18X,
viii. an attenuation of Mercurius vivus at a dilution of 10X,
ix. an attenuation of Hypericum at a dilution of 10X,
x. an attenuation of Silicea at a dilution of 8X, and
xi. an attenuation of Ignatia amara at a dilution of 30X; and
b. the third composition comprises:
i. an attenuation of Calendula officinalis at a dilution of 1X,
ii. an attenuation of Echinacea purpurea at a dilution of 1X,
iii. an attenuation of Plantago major at a dilution of 1X, and
iv. an attenuation of Azadirachta indica at a dilution of 1X.

40. (canceled)

41. (canceled)

42. (canceled)

43. (canceled)

44. (canceled)

45. (canceled)

46. (canceled)

47. (canceled)

48. (canceled)

49. A kit comprising a first composition formulated as a spray, a second composition formulated as a rinse, and at least one container; wherein
a. the first composition comprises:
i. an attenuation of Arnica montana at a dilution of 3X,
ii. an attenuation of Argentum nitricum at a dilution of 30X,
iii. an attenuation of Calcarea carbonica at a dilution of 6X,
iv. an attenuation of Calendula officinalis at a dilution of 3X,
v. an attenuation of Capsicum annum at a dilution of 5X,
vi. an attenuation of Causticum at a dilution of 6X,
vii. an attenuation of Chamomilla at a dilution of 5X,
viii. an attenuation of Echinacea angustifolia at a dilution of 2X,
ix. an attenuation of Hepar sulphuris calcaneum at a dilution of 6X,
x. an attenuation of Lachesis at a dilution of 12X,
xi. an attenuation of Lac caninum at a dilution of 8X,
xii. an attenuation of Phytolacca at a dilution of 5X, and
xiii. an attenuation of Rhus-toxicodendron at a dilution of 12X, and
b. the second composition comprises:
i. an attenuation of Calendua officinalis at a dilution of 1X,
ii. an attenuation of Echinacea purpurea at a dilution of 1X,
iii. an attenuation of Plantago major at a dilution of 1X, and
iv. an attenuation of Azadirachta indica/Neem at a dilution of 1X.

50. A kit comprising a first composition formulated as a gel, a second composition formulated as a rinse, and at least one container; wherein
a. the first composition comprises:
i. an attenuation of Hepar sulphuris calcaneum at a dilution of 8X,
ii. an attenuation of Calendula officinalis at a dilution of 3X,
iii. an attenuation of Chamomilla at a dilution of 5X,
iv. an attenuation of Aconitum napellus at a dilution of 3X,
v. an attenuation of Echinacea angustifolia or an attenuation of Echinacea purpurea at a dilution of 3X,
vi. an attenuation of Ruta graveolens at a dilution of 12X,
vii. an attenuation of Arnica montana at a dilution of 3X, 6X, or 18X,
viii. an attenuation of Mercurius vivus at a dilution of 10X,
ix. an attenuation of Hypericum at a dilution of 10X,
x. an attenuation of Gelsemium at a dilution of 30X, and
xi. an attenuation of Ignatia amara at a dilution of 30X; and
b. the second composition comprises:
i. an attenuation of Azadirachta indica at a dilution of 6X,
ii. an attenuation of Calendula officinalis at a dilution of 1X,
iii. an attenuation of Echinacea purpurea at a dilution of 1X,
iv. an attenuation of Plantago major at a dilution of 1X,
v. an attenuation of Apis mellifica at a dilution of 12X,
vi. an attenuation of Arsenicum album at a dilution of 30X,
vii. an attenuation of Aristolochia clematitis at a dilution of 5X,
viii. an attenuation of Croton lechleri at a dilution of 1X,
ix. an attenuation of Okoubaka aubrevillei at a dilution of 6X,
x. an attenuation of Phosphorous at a dilution of 12X,
xi. an attenuation of Xanthoxylum at a dilution of 1X,
xii. an attenuation of Strontium carbinicum at a dilution of 30X, and
xiii. an attenuation of Thuja occidentalis at a dilution of 3X.

51. A composition formulated as a toothpaste comprising:
a. an attenuation of Baobab at a dilution of 1X,
b. an attenuation of Calendula officinalis at a dilution of 1X,
c. an attenuation of Copaiba oil at a dilution of 1X,
d. an attenuation of Echinacea angustifolia at a dilution of 3X,
e. Baking Soda (non-aluminum NaHCO3),
f. Bentonite Clay,
g. Castile soap,
h. Coconut Oil,
i. Colloidal silver (silver hydrosol),
j. CoQ10,
k. Coral Calcium,
l. Probiotics,
m. Silica,
n. Tumeric, and
o. Xylitol.

52. A composition formulated as a balm comprising:
a. Aloe Vera Butter
b. an attenuation of Apis mellifica at a dilution of 8X,
c. an attenuation of Arsenicum album at a dilution of 8X,
d. an attenuation of Calendula officinalis at a dilution of 2X,
e. an attenuation of Cantharis at a dilution of 5X,
f. an attenuation of Carbolicum acidum at a dilution of 10X,
g. an attenuation of Causticum at a dilution of 5X,
h. an attenuation of Graphites at a dilution of 6X,
i. an attenuation of Hypericum at a dilution of 5X,
j. an attenuation of Plantago major at a dilution of 2X, and
k. an attenuation of Thiosinaminum at a dilution of 3X.
